SEQUENCE LISTING (241 sequences)

SEQ ID NO: 1 - protein, 4 aa, synthetic construct
  REGION 1..4: note = Synthetic Construct
LLQG

SEQ ID NO: 2-3 - skipped (2 entries)

SEQ ID NO: 4 - protein, 83 aa, synthetic construct
  REGION 1..83: note = Synthetic Construct
DSATHIKFSKRDEDGKELAGATMELRDSSGKTISTWISDGQVKDFYLYPGKYTFVETAAPDGYEVATAITFTVNEQGQVTVNG

SEQ ID NO: 5 - protein, 13 aa, synthetic construct
  REGION 1..13: note = Synthetic Construct
AHIVMVAAYKPTK

SEQ ID NO: 6 - protein, 34 aa, synthetic construct
  REGION 1..34: note = Synthetic Construct
MQIFVKTLTGKTITLEVEPSDTIENVKAKIQDKE

SEQ ID NO: 7 - protein, 42 aa, synthetic construct
  REGION 1..42: note = Synthetic Construct
GIPPDQQRLIFAGKQLEDGRTLSDYNIQKESTLHLVLRLRGG

SEQ ID NO: 8 - protein, 160 aa, synthetic construct
  REGION 1..160: note = Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 9 - protein, 70 aa, synthetic construct
  REGION 1..70: note = Synthetic Construct
EAEEDGDLQCLCVKTTSQVRPRHITSLEVIKAGPHCPTAQLIATLKNGRKICLDLQAPLYKKIIKKLLES

SEQ ID NO: 10 - protein, 78 aa, synthetic construct
  REGION 1..78: note = Synthetic Construct
AGPAAAVLRELRCVCLQTTQGVHPKMISNLQVFAIGPQCSKVEVVASLKNGKEICLDPEAPFLKKVIQKILDGGNKEN

SEQ ID NO: 11 - protein, 219 aa, synthetic construct
  REGION 1..219: note = Synthetic Construct
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 12 - protein, 328 aa, synthetic construct
  REGION 1..328: note = Synthetic Construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 13 - protein, 30 aa, synthetic construct
  REGION 1..30: note = Synthetic Construct
ENKQVEEILRLEKEIEDLQRMKEQQELSLT

SEQ ID NO: 14 - protein, 61 aa, synthetic construct
  REGION 1..61: note = Synthetic Construct
GAMDPEFTKNALNVVKNDLIAKVDQLSGEQEVLRGELEAAKQAKVKLENRIKELEEELKRV

SEQ ID NO: 15 - protein, 80 aa, synthetic construct
  REGION 1..80: note = Synthetic Construct
GSMTISNMEADMNRLLKQREELTKRREKLSKRREKIVKENGEGDKNVANINEEMESLTANIDYINDSISDCQANIMQMEE

SEQ ID NO: 16 - protein, 51 aa, synthetic construct
  REGION 1..51: note = Synthetic Construct
GSDYEFLKSWTVEDLQKRLLALDPMMEQEIEEIRQKYQSKRQPILDAIEAK

SEQ ID NO: 17 - protein, 55 aa, synthetic construct
  REGION 1..55: note = Synthetic Construct
GSEVEWDAFSIPELQNFLTILEKEEQDKIQQVQKKYDKFRQKLEEALRESQGKPG

SEQ ID NO: 18 - protein, 14 aa, synthetic construct
  REGION 1..14: note = Synthetic Construct
SNYSMVNTTNMTSA

SEQ ID NO: 19 - protein, 8 aa, synthetic construct
  REGION 1..8: note = Synthetic Construct
LSGRSDNH

SEQ ID NO: 20 - protein, 218 aa, synthetic construct
  REGION 1..218: note = Synthetic Construct
QSCATGPRNCKDLLDRGYFRSGWHTIYLPDCRPLTVLCDMDTDGGGWTVFQRRMDGSVDFYRDWAAYKQGFGSQLGEFWLGNDNIHALTAQGSSELRTDLVDFEGNHQFAKYKSFKVADEAEKYKLVLGAFVGGSAGNSLTGHNNNFFSTKDQDNDVSSSNCAEKFQGAWWYADCHASNLNGLYLMGPHESYANGINWSAAKGYKYSYKVSEMKVRPA

SEQ ID NO: 21 - protein, 460 aa, synthetic construct
  REGION 1..460: note = Synthetic Construct
LLQGLSGRSDNHQVQLKQSGPGLVQPSQSLSITCTVSGFSLTNYGVHWVRQSPGKGLEWLGVIWSGGNTDYNTPFTSRLSINKDNSKSQVFFKMNSLQSEDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 225 aa, synthetic construct
  REGION 1..225: note = Synthetic Construct
GKGLSGRSDNHDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 464 aa, synthetic construct
  REGION 1..464: note = Synthetic Construct
LLQGLSGRSDNHGGGSQVQLKQSGPGLVQPSQSLSITCTVSGFSLTNYGVHWVRQSPGKGLEWLGVIWSGGNTDYNTPFTSRLSINKDNSKSQVFFKMNSLQSEDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 229 aa, synthetic construct
  REGION 1..229: note = Synthetic Construct
GKGLSGRSDNHGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 460 aa, synthetic construct
  REGION 1..460: note = Synthetic Construct
SCLSGRSDNHGGQVQLKQSGPGLVQPSQSLSITCTVSGFSLTNYGVHWVRQSPGKGLEWLGVIWSGGNTDYNTPFTSRLSINKDNSKSQVFFKMNSLQSEDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 226 aa, synthetic construct
  REGION 1..226: note = Synthetic Construct
SCLSGRSDNHGGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 27 - protein, 462 aa, synthetic construct
  REGION 1..462: note = Synthetic Construct
SCLSGRSDNHGGGSQVQLKQSGPGLVQPSQSLSITCTVSGFSLTNYGVHWVRQSPGKGLEWLGVIWSGGNTDYNTPFTSRLSINKDNSKSQVFFKMNSLQSEDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 228 aa, synthetic construct
  REGION 1..228: note = Synthetic Construct
SCLSGRSDNHGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 29 - protein, 239 aa, synthetic construct
  REGION 1..239: note = Synthetic Construct
LLQGLSGRSDNHGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 30 - protein, 229 aa, synthetic construct
  REGION 1..229: note = Synthetic Construct
GKGLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 237 aa, synthetic construct
  REGION 1..237: note = Synthetic Construct
LLQGLSGRSDNHGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 32 - protein, 230 aa, synthetic construct
  REGION 1..230: note = Synthetic Construct
GKGLSGRSDNHGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 234 aa, synthetic construct
  REGION 1..234: note = Synthetic Construct
LLQGLSGRSDNHGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 34 - protein, 233 aa, synthetic construct
  REGION 1..233: note = Synthetic Construct
GKGLSGRSDNHGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 114 aa, synthetic construct
  REGION 1..114: note = Synthetic Construct
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTV

SEQ ID NO: 36 - protein, 107 aa, synthetic construct
  REGION 1..107: note = Synthetic Construct
EIVLTQSPGTLSLSPGERATLSCRASQSVSSSYLAWYQQKPGQAPRLLIYYASSRATGIPDRFSGSGSGTDFTLTISRLEPEDFAVYYCQQTGRIPPTFGQGTKVEI

SEQ ID NO: 37 - protein, 616 aa, synthetic construct
  REGION 1..616: note = Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNLSGRSDNHQVQLKQSGPGLVQPSQSLSITCTVSGFSLTNYGVHWVRQSPGKGLEWLGVIWSGGNTDYNTPFTSRLSINKDNSKSQVFFKMNSLQSEDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 382 aa, synthetic construct
  REGION 1..382: note = Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNLSGRSDNHDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 490 aa, synthetic construct
  REGION 1..490: note = Synthetic Construct
MQIFVKTLTGKTITLEVEPSDTIENVKAKIQDKELSGRSDNHQVQLKQSGPGLVQPSQSLSITCTVSGFSLTNYGVHWVRQSPGKGLEWLGVIWSGGNTDYNTPFTSRLSINKDNSKSQVFFKMNSLQSEDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 264 aa, synthetic construct
  REGION 1..264: note = Synthetic Construct
GIPPDQQRLIFAGKQLEDGRTLSDYNIQKESTLHLVLRLRGGLSGRSDNHDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 526 aa, synthetic construct
  REGION 1..526: note = Synthetic Construct
EAEEDGDLQCLCVKTTSQVRPRHITSLEVIKAGPHCPTAQLIATLKNGRKICLDLQAPLYKKIIKKLLESLSGRSDNHQVQLKQSGPGLVQPSQSLSITCTVSGFSLTNYGVHWVRQSPGKGLEWLGVIWSGGNTDYNTPFTSRLSINKDNSKSQVFFKMNSLQSEDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 292 aa, synthetic construct
  REGION 1..292: note = Synthetic Construct
EAEEDGDLQCLCVKTTSQVRPRHITSLEVIKAGPHCPTAQLIATLKNGRKICLDLQAPLYKKIIKKLLESLSGRSDNHDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 486 aa, synthetic construct
  REGION 1..486: note = Synthetic Construct
ENKQVEEILRLEKEIEDLQRMKEQQELSLTLSGRSDNHQVQLKQSGPGLVQPSQSLSITCTVSGFSLTNYGVHWVRQSPGKGLEWLGVIWSGGNTDYNTPFTSRLSINKDNSKSQVFFKMNSLQSEDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 252 aa, synthetic construct
  REGION 1..252: note = Synthetic Construct
ENKQVEEILRLEKEIEDLQRMKEQQELSLTLSGRSDNHDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 572 aa, synthetic construct
  REGION 1..572: note = Synthetic Construct
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVLSGRSDNHVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 46 - protein, 328 aa, synthetic construct
  REGION 1..328: note = Synthetic Construct
EIVLTQSPGTLSLSPGERATLSCRASQSVSSSYLAWYQQKPGQAPRLLIYYASSRATGIPDRFSGSGSGTDFTLTISRLEPEDFAVYYCQQTGRIPPTFGQGTKVEILSGRSDNHIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 666 aa, synthetic construct
  REGION 1..666: note = Synthetic Construct
QSCATGPRNCKDLLDRGYFRSGWHTIYLPDCRPLTVLCDMDTDGGGWTVFQRRMDGSVDFYRDWAAYKQGFGSQLGEFWLGNDNIHALTAQGSSELRTDLVDFEGNHQFAKYKSFKVADEAEKYKLVLGAFVGGSAGNSLTGHNNNFFSTKDQDNDVSSSNCAEKFQGAWWYADCHASNLNGLYLMGPHESYANGINWSAAKGYKYSYKVSEMKVRPAQVQLKQSGPGLVQPSQSLSITCTVSGFSLTNYGVHWVRQSPGKGLEWLGVIWSGGNTDYNTPFTSRLSINKDNSKSQVFFKMNSLQSEDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 227 aa, synthetic construct
  REGION 1..227: note = Synthetic Construct
SNYSMVNTTNMTSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 674 aa, synthetic construct
  REGION 1..674: note = Synthetic Construct
QSCATGPRNCKDLLDRGYFRSGWHTIYLPDCRPLTVLCDMDTDGGGWTVFQRRMDGSVDFYRDWAAYKQGFGSQLGEFWLGNDNIHALTAQGSSELRTDLVDFEGNHQFAKYKSFKVADEAEKYKLVLGAFVGGSAGNSLTGHNNNFFSTKDQDNDVSSSNCAEKFQGAWWYADCHASNLNGLYLMGPHESYANGINWSAAKGYKYSYKVSEMKVRPALSGRSDNHQVQLKQSGPGLVQPSQSLSITCTVSGFSLTNYGVHWVRQSPGKGLEWLGVIWSGGNTDYNTPFTSRLSINKDNSKSQVFFKMNSLQSEDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 235 aa, synthetic construct
  REGION 1..235: note = Synthetic Construct
SNYSMVNTTNMTSLSGRSDNHDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51-53 - skipped (3 entries)

SEQ ID NO: 54 - protein, 11 aa, synthetic construct
  REGION 1..11: note = Synthetic Construct
VLNLAQSKNFH

SEQ ID NO: 55 - protein, 11 aa, synthetic construct
  REGION 1..11: note = Synthetic Construct
APALQPTQGAM

SEQ ID NO: 56 - protein, 9 aa, synthetic construct
  REGION 1..9: note = Synthetic Construct
IPKEQKYSF

SEQ ID NO: 57 - protein, 13 aa, synthetic construct
  REGION 1..13: note = Synthetic Construct
MGGSPLAQSHGGS

SEQ ID NO: 58 - protein, 15 aa, synthetic construct
  REGION 1..15: note = Synthetic Construct
KETAAAKFERQHMDS

SEQ ID NO: 59 - protein, 12 aa, synthetic construct
  REGION 1..12: note = Synthetic Construct
TEYGLFQINNDS

SEQ ID NO: 60 - protein, 6 aa, synthetic construct
  REGION 1..6: note = Synthetic Construct
APQQEA

SEQ ID NO: 61 - protein, 7 aa, synthetic construct
  REGION 1..7: note = Synthetic Construct
EDGFFKI

SEQ ID NO: 62 - protein, 9 aa, synthetic construct
  REGION 1..9: note = Synthetic Construct
EEQYNSTYR

SEQ ID NO: 63 - protein, 34 aa, synthetic construct
  REGION 1..34: note = Synthetic Construct
KLEALEGKLEALEKLEALEGKLEALEGKLEALEG

SEQ ID NO: 64 - protein, 30 aa, synthetic construct
  REGION 1..30: note = Synthetic Construct
DNIEQKIDDIDHEIADLQAKRTRKVQQHPR

SEQ ID NO: 65 - protein, 30 aa, synthetic construct
  REGION 1..30: note = Synthetic Construct
RPHQQVLRTRKAQLDAIEHDIDDIKQEIND

SEQ ID NO: 66 - protein, 11 aa, synthetic construct
  REGION 1..11: note = Synthetic Construct
RRRRRRRRRRR

SEQ ID NO: 67 - protein, 11 aa, synthetic construct
  REGION 1..11: note = Synthetic Construct
EEEEEEEEEEE

SEQ ID NO: 68 - skipped

SEQ ID NO: 69 - protein, 20 aa, synthetic construct
  REGION 1..20: note = Synthetic Construct
VNNTIVWTNSSLNQNMTNGT

SEQ ID NO: 70 - protein, 15 aa, synthetic construct
  REGION 1..15: note = Synthetic Construct
FNQTQPERGDNNLTR

SEQ ID NO: 71 - protein, 11 aa, synthetic construct
  REGION 1..11: note = Synthetic Construct
GNSTHSRNNSY

SEQ ID NO: 72 - protein, 9 aa, synthetic construct
  REGION 1..9: note = Synthetic Construct
EEQYNSTYR

SEQ ID NO: 73 - protein, 9 aa, synthetic construct
  REGION 1..9: note = Synthetic Construct
EEQYNSTFR

SEQ ID NO: 74 - skipped

SEQ ID NO: 75 - protein, 4 aa, synthetic construct
  REGION 1..4: note = Synthetic Construct
GGGG

SEQ ID NO: 76 - skipped

SEQ ID NO: 77 - protein, 5 aa, synthetic construct
  REGION 1..5: note = Synthetic Construct
LLQGG

SEQ ID NO: 78 - protein, 6 aa, synthetic construct
  REGION 1..6: note = Synthetic Construct
LSLSQG

SEQ ID NO: 79 - protein, 8 aa, synthetic construct
  REGION 1..8: note = Synthetic Construct
GGGLLQGG

SEQ ID NO: 80 - protein, 5 aa, synthetic construct
  REGION 1..5: note = Synthetic Construct
GLLQG

SEQ ID NO: 81 - skipped

SEQ ID NO: 82 - protein, 10 aa, synthetic construct
  REGION 1..10: note = Synthetic Construct
GSPLAQSHGG

SEQ ID NO: 83 - protein, 7 aa, synthetic construct
  REGION 1..7: note = Synthetic Construct
GLLQGGG

SEQ ID NO: 84 - protein, 6 aa, synthetic construct
  REGION 1..6: note = Synthetic Construct
GLLQGG

SEQ ID NO: 85 - protein, 4 aa, synthetic construct
  REGION 1..4: note = Synthetic Construct
GLLQ

SEQ ID NO: 86 - protein, 8 aa, synthetic construct
  REGION 1..8: note = Synthetic Construct
LLQLLQGA

SEQ ID NO: 87 - protein, 5 aa, synthetic construct
  REGION 1..5: note = Synthetic Construct
LLQGA

SEQ ID NO: 88 - protein, 7 aa, synthetic construct
  REGION 1..7: note = Synthetic Construct
LLQYQGA

SEQ ID NO: 89 - protein, 6 aa, synthetic construct
  REGION 1..6: note = Synthetic Construct
LLQGSG

SEQ ID NO: 90 - protein, 6 aa, synthetic construct
  REGION 1..6: note = Synthetic Construct
LLQYQG

SEQ ID NO: 91 - protein, 7 aa, synthetic construct
  REGION 1..7: note = Synthetic Construct
LLQLLQG

SEQ ID NO: 92 - protein, 5 aa, synthetic construct
  REGION 1..5: note = Synthetic Construct
SLLQG

SEQ ID NO: 93 - protein, 5 aa, synthetic construct
  REGION 1..5: note = Synthetic Construct
LLQLQ

SEQ ID NO: 94 - protein, 6 aa, synthetic construct
  REGION 1..6: note = Synthetic Construct
LLQLLQ

SEQ ID NO: 95 - protein, 5 aa, synthetic construct
  REGION 1..5: note = Synthetic Construct
LLQGR

SEQ ID NO: 96 - protein, 6 aa, synthetic construct
  REGION 1..6: note = Synthetic Construct
LLQGPP

SEQ ID NO: 97 - protein, 6 aa, synthetic construct
  REGION 1..6: note = Synthetic Construct
LLQGPA

SEQ ID NO: 98 - protein, 8 aa, synthetic construct
  REGION 1..8: note = Synthetic Construct
GGLLQGPP

SEQ ID NO: 99 - protein, 7 aa, synthetic construct
  REGION 1..7: note = Synthetic Construct
GGLLQGA

SEQ ID NO: 100 - protein, 7 aa, synthetic construct
  REGION 1..7: note = Synthetic Construct
LLQGPGK

SEQ ID NO: 101 - protein, 6 aa, synthetic construct
  REGION 1..6: note = Synthetic Construct
LLQGPG

SEQ ID NO: 102 - protein, 5 aa, synthetic construct
  REGION 1..5: note = Synthetic Construct
LLQGP

SEQ ID NO: 103 - protein, 4 aa, synthetic construct
  REGION 1..4: note = Synthetic Construct
LLQP

SEQ ID NO: 104 - protein, 6 aa, synthetic construct
  REGION 1..6: note = Synthetic Construct
LLQPGK

SEQ ID NO: 105 - protein, 7 aa, synthetic construct
  REGION 1..7: note = Synthetic Construct
LLQAPGK

SEQ ID NO: 106 - protein, 7 aa, synthetic construct
  REGION 1..7: note = Synthetic Construct
LLQGAPG

SEQ ID NO: 107 - protein, 6 aa, synthetic construct
  REGION 1..6: note = Synthetic Construct
LLQGAP

SEQ ID NO: 108 - protein, 6 aa, synthetic construct
  REGION 1..6: note = Synthetic Construct
LLQLQG

SEQ ID NO: 109 - protein, 5 aa, synthetic construct
  REGION 1..5: note = Synthetic Construct
LLQGA

SEQ ID NO: 110 - skipped

SEQ ID NO: 111 - protein, 7 aa, synthetic construct
  REGION 1..7: note = Synthetic Construct
GGLLQGA

SEQ ID NO: 112 - protein, 6 aa, synthetic construct
  REGION 1..6: note = Synthetic Construct
LLQGPA

SEQ ID NO: 113 - protein, 6 aa, synthetic construct
  REGION 1..6: note = Synthetic Construct
LLQGPP

SEQ ID NO: 114 - protein, 8 aa, synthetic construct
  REGION 1..8: note = Synthetic Construct
GGLLQGPP

SEQ ID NO: 115 - protein, 6 aa, synthetic construct
  REGION 1..6: note = Synthetic Construct
LLQGWG

SEQ ID NO: 116 - protein, 4 aa, synthetic construct
  REGION 1..4: note = Synthetic Construct
LLQG

SEQ ID NO: 117 - protein, 6 aa, synthetic construct
  REGION 1..6: note = Synthetic Construct
LLQYQG

SEQ ID NO: 118 - protein, 7 aa, synthetic construct
  REGION 1..7: note = Synthetic Construct
LLQLLQG

SEQ ID NO: 119 - protein, 6 aa, synthetic construct
  REGION 1..6: note = Synthetic Construct
LLQLQG

SEQ ID NO: 120 - protein, 6 aa, synthetic construct
  REGION 1..6: note = Synthetic Construct
LLQLLQ

SEQ ID NO: 121 - protein, 5 aa, synthetic construct
  REGION 1..5: note = Synthetic Construct
LLQLQ

SEQ ID NO: 122 - protein, 5 aa, synthetic construct
  REGION 1..5: note = Synthetic Construct
LLQGR

SEQ ID NO: 123 - protein, 7 aa, synthetic construct
  REGION 1..7: note = Synthetic Construct
LLQYQGA

SEQ ID NO: 124 - protein, 5 aa, synthetic construct
  REGION 1..5: note = Synthetic Construct
SLLQG

SEQ ID NO: 125 - protein, 8 aa, synthetic construct
  REGION 1..8: note = Synthetic Construct
LLQLLQGA

SEQ ID NO: 126 - protein, 6 aa, synthetic construct
  REGION 1..6: note = Synthetic Construct
LLQGPP

SEQ ID NO: 127 - protein, 6 aa, synthetic construct
  REGION 1..6: note = Synthetic Construct
QVQLKE

SEQ ID NO: 128 - protein, 5 aa, synthetic construct
  REGION 1..5: note = Synthetic Construct
VQLKE

SEQ ID NO: 129 - protein, 7 aa, synthetic construct
  REGION 1..7: note = Synthetic Construct
GGLLQGG

SEQ ID NO: 130 - protein, 6 aa, synthetic construct
  REGION 1..6: note = Synthetic Construct
TVQQEL

SEQ ID NO: 131 - protein, 7 aa, synthetic construct
  REGION 1..7: note = Synthetic Construct
GQQQTPY

SEQ ID NO: 132 - protein, 7 aa, synthetic construct
  REGION 1..7: note = Synthetic Construct
GLQQASV

SEQ ID NO: 133 - protein, 7 aa, synthetic construct
  REGION 1..7: note = Synthetic Construct
WQTPMNS

SEQ ID NO: 134 - protein, 12 aa, synthetic construct
  REGION 1..12: note = Synthetic Construct
WQHPLHDWFDLV

SEQ ID NO: 135 - protein, 12 aa, synthetic construct
  REGION 1..12: note = Synthetic Construct
FQQPLDPWTSPI

SEQ ID NO: 136 - protein, 12 aa, synthetic construct
  REGION 1..12: note = Synthetic Construct
HQSYVDPWMLDH

SEQ ID NO: 137 - protein, 12 aa, synthetic construct
  REGION 1..12: note = Synthetic Construct
REQLYLDYNVFS

SEQ ID NO: 138 - protein, 13 aa, synthetic construct
  REGION 1..13: note = Synthetic Construct
MWQKLPLVVHWPT

SEQ ID NO: 139 - protein, 12 aa, synthetic construct
  REGION 1..12: note = Synthetic Construct
THMYQSIYVPDI

SEQ ID NO: 140 - protein, 12 aa, synthetic construct
  REGION 1..12: note = Synthetic Construct
LLQGLSGRSDNH

SEQ ID NO: 141 - protein, 11 aa, synthetic construct
  REGION 1..11: note = Synthetic Construct
GKGLSGRSDNH

SEQ ID NO: 142 - protein, 16 aa, synthetic construct
  REGION 1..16: note = Synthetic Construct
LLQGLSGRSDNHGGGS

SEQ ID NO: 143 - protein, 15 aa, synthetic construct
  REGION 1..15: note = Synthetic Construct
GKGLSGRSDNHGGGS

SEQ ID NO: 144 - protein, 10 aa, synthetic construct
  REGION 1..10: note = Synthetic Construct
SCLSGRSDNH

SEQ ID NO: 145 - protein, 12 aa, synthetic construct
  REGION 1..12: note = Synthetic Construct
SCLSGRSDNHGG

SEQ ID NO: 146 - protein, 14 aa, synthetic construct
  REGION 1..14: note = Synthetic Construct
SCLSGRSDNHGGGS

SEQ ID NO: 147 - protein, 6 aa, synthetic construct
  REGION 1..6: note = Synthetic Construct
RQARVV

SEQ ID NO: 148 - protein, 6 aa, synthetic construct
  REGION 1..6: note = Synthetic Construct
  VARIANT 1: note = Xaa = Gly or Lys
XSGRSA

SEQ ID NO: 149-153 - skipped (5 entries)

SEQ ID NO: 154 - protein, 8 aa, synthetic construct
  REGION 1..8: note = Synthetic Construct
  VARIANT 6: note = Xaa = Asp, Val, or Ala
LSGRSXNH

SEQ ID NO: 155 - skipped

SEQ ID NO: 156 - protein, 4 aa, synthetic construct
  REGION 1..4: note = Synthetic Construct
AANL

SEQ ID NO: 157 - protein, 4 aa, synthetic construct
  REGION 1..4: note = Synthetic Construct
ATNL

SEQ ID NO: 158 - skipped

SEQ ID NO: 159 - protein, 6 aa, synthetic construct
  REGION 1..6: note = Synthetic Construct
  VARIANT 1: note = Xaa = Gly or Lys
XSGRSA

SEQ ID NO: 160 - skipped

SEQ ID NO: 161 - protein, 5 aa, synthetic construct
  REGION 1..5: note = Synthetic Construct
SGRSA

SEQ ID NO: 162 - skipped

SEQ ID NO: 163 - protein, 13 aa, synthetic construct
  REGION 1..13: note = Synthetic Construct
RKSSIIIRMRDWL

SEQ ID NO: 164 - protein, 8 aa, synthetic construct
  REGION 1..8: note = Synthetic Construct
  VARIANT 4: note = Xaa = Lys or Arg
PRFXIIGG

SEQ ID NO: 165 - protein, 7 aa, synthetic construct
  REGION 1..7: note = Synthetic Construct
SRPLALR

SEQ ID NO: 166 - protein, 7 aa, synthetic construct
  REGION 1..7: note = Synthetic Construct
SRPANLR

SEQ ID NO: 167 - skipped

SEQ ID NO: 168 - protein, 8 aa, synthetic construct
  REGION 1..8: note = Synthetic Construct
DVAQFVLT

SEQ ID NO: 169 - skipped

SEQ ID NO: 170 - protein, 8 aa, synthetic construct
  REGION 1..8: note = Synthetic Construct
  VARIANT 6: note = Xaa = Val or Trp
HRPRGXTN

SEQ ID NO: 171 - skipped

SEQ ID NO: 172 - protein, 6 aa, synthetic construct
  REGION 1..6: note = Synthetic Construct
PLGLAG

SEQ ID NO: 173 - protein, 8 aa, synthetic construct
  REGION 1..8: note = Synthetic Construct
  VARIANT 6: note = Xaa = Val or Trp
HRPRGXTN

SEQ ID NO: 174 - skipped

SEQ ID NO: 175 - protein, 8 aa, synthetic construct
  REGION 1..8: note = Synthetic Construct
GPLGIAGQ

SEQ ID NO: 176 - protein, 6 aa, synthetic construct
  REGION 1..6: note = Synthetic Construct
PVGLIG

SEQ ID NO: 177 - protein, 8 aa, synthetic construct
  REGION 1..8: note = Synthetic Construct
HPVGLLAR

SEQ ID NO: 178 - protein, 6 aa, synthetic construct
  REGION 1..6: note = Synthetic Construct
PLGLWA

SEQ ID NO: 179 - skipped

SEQ ID NO: 180 - protein, 5 aa, synthetic construct
  REGION 1..5: note = Synthetic Construct
SSKLQ

SEQ ID NO: 181 - protein, 7 aa, synthetic construct
  REGION 1..7: note = Synthetic Construct
SRKSQQY

SEQ ID NO: 182 - protein, 7 aa, synthetic construct
  REGION 1..7: note = Synthetic Construct
GQKGQHY

SEQ ID NO: 183 - protein, 7 aa, synthetic construct
  REGION 1..7: note = Synthetic Construct
KGISSQY

SEQ ID NO: 184 - skipped

SEQ ID NO: 185 - protein, 6 aa, synthetic construct
  REGION 1..6: note = Synthetic Construct
  VARIANT 3: note = Xaa = Tyr or Phe
  VARIANT 6: note = Xaa = Gly or Ser
SSXYSX

SEQ ID NO: 186 - skipped

SEQ ID NO: 187 - protein, 6 aa, synthetic construct
  REGION 1..6: note = Synthetic Construct
  VARIANT 1: note = Xaa = Arg or Met
  VARIANT 3: note = Xaa = Ala or His
XEXVVY

SEQ ID NO: 188 - protein, 6 aa, synthetic construct
  REGION 1..6: note = Synthetic Construct
VADCAQ

SEQ ID NO: 189 - protein, 9 aa, synthetic construct
  REGION 1..9: note = Synthetic Construct
APEEIMDRQ

SEQ ID NO: 190 - protein, 16 aa, synthetic construct
  REGION 1..16: note = Synthetic Construct
IVSARMAPEEIIMDRQ

SEQ ID NO: 191 - protein, 12 aa, synthetic construct
  REGION 1..12: note = Synthetic Construct
GIATFCMLMPEQ

SEQ ID NO: 192 - skipped

SEQ ID NO: 193 - protein, 6 aa, synthetic construct
  REGION 1..6: note = Synthetic Construct
VADCAQ

SEQ ID NO: 194 - protein, 9 aa, synthetic construct
  REGION 1..9: note = Synthetic Construct
APEEIMDRQ

SEQ ID NO: 195 - protein, 16 aa, synthetic construct
  REGION 1..16: note = Synthetic Construct
IVSARMAPEEIIMDRQ

SEQ ID NO: 196 - protein, 12 aa, synthetic construct
  REGION 1..12: note = Synthetic Construct
GIATFCMLMPEQ

SEQ ID NO: 197 - protein, 4 aa, synthetic construct
  REGION 1..4: note = Synthetic Construct
IEGR

SEQ ID NO: 198 - protein, 4 aa, synthetic construct
  REGION 1..4: note = Synthetic Construct
IDGR

SEQ ID NO: 199 - protein, 7 aa, synthetic construct
  REGION 1..7: note = Synthetic Construct
GGSIDGR

SEQ ID NO: 200-202 - skipped (3 entries)

SEQ ID NO: 203 - protein, 30 aa, synthetic construct
  REGION 1..30: note = Synthetic Construct
WEAALAEALAEALAEHLAEALAEALEALAA

SEQ ID NO: 204-205 - skipped (2 entries)

SEQ ID NO: 206 - protein, 5 aa, synthetic construct
  REGION 1..5: note = Synthetic Construct
GGGGS

SEQ ID NO: 207 - protein, 4 aa, synthetic construct
  REGION 1..4: note = Synthetic Construct
GGSG

SEQ ID NO: 208 - protein, 4 aa, synthetic construct
  REGION 1..4: note = Synthetic Construct
SGGG

SEQ ID NO: 209 - protein, 4 aa, synthetic construct
  REGION 1..4: note = Synthetic Construct
GSGS

SEQ ID NO: 210 - protein, 6 aa, synthetic construct
  REGION 1..6: note = Synthetic Construct
GSGSGS

SEQ ID NO: 211 - protein, 8 aa, synthetic construct
  REGION 1..8: note = Synthetic Construct
GSGSGSGS

SEQ ID NO: 212 - protein, 10 aa, synthetic construct
  REGION 1..10: note = Synthetic Construct
GSGSGSGSGS

SEQ ID NO: 213 - protein, 12 aa, synthetic construct
  REGION 1..12: note = Synthetic Construct
GSGSGSGSGSGS

SEQ ID NO: 214 - protein, 6 aa, synthetic construct
  REGION 1..6: note = Synthetic Construct
GGSGGS

SEQ ID NO: 215 - protein, 9 aa, synthetic construct
  REGION 1..9: note = Synthetic Construct
GGSGGSGGS

SEQ ID NO: 216 - protein, 12 aa, synthetic construct
  REGION 1..12: note = Synthetic Construct
GGSGGSGGSGGS

SEQ ID NO: 217 - protein, 8 aa, synthetic construct
  REGION 1..8: note = Synthetic Construct
GGSGGGSG

SEQ ID NO: 218 - protein, 12 aa, synthetic construct
  REGION 1..12: note = Synthetic Construct
GGSGGGSGGGSG

SEQ ID NO: 219 - protein, 5 aa, synthetic construct
  REGION 1..5: note = Synthetic Construct
  VARIANT 1..5: note = Can be present in repeat of at least one and up to 10
GGGGS

SEQ ID NO: 220 - skipped

SEQ ID NO: 221 - protein, 6 aa, synthetic construct
  REGION 1..6: note = Synthetic Construct
  VARIANT 2..6: note = Can be present in repeat of at least one and up to 10
AEAAAK

SEQ ID NO: 222 - skipped

SEQ ID NO: 223 - protein, 4 aa, synthetic construct
  REGION 1..4: note = Synthetic Construct
GGGS

SEQ ID NO: 224 - protein, 232 aa, synthetic construct
  REGION 1..232: note = Synthetic Construct
LLQGPLGLAGGSGGGSEAKLQESGPVLVKPGASVKMSCKASGYTFTDYYMNWVKQSHGKSLEWIGVINPYNGDTSYNQKFKGKATLTVDKSSSTAYMELNSLTSEDSAVYYCARYYGSWFAYWGQGTLITVSTAKTTPPSVYPLAPGCGDTTGSSVTLGCLVKGYFPESVTVTWNSGSLSSSVHTFPALLQSGLYTMSSSVTVPSSTWPSETVTCSVAHPASSTTVDKKLEP

SEQ ID NO: 225 - protein, 234 aa, synthetic construct
  REGION 1..234: note = Synthetic Construct
GKGLSGRSDNHGGGSDIVMTQTTLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKRADAAPTVSIFPPSSEQLTSGGASVVCFLNNFYPKDINVKWKIDGSERQNGVLNSWTDQNSKDSTYSMSSTLTLTKDEYERHNSYTCEATHKTSTSPIVKSFNRNEC

SEQ ID NO: 226 - protein, 6 aa, synthetic construct
  REGION 1..6: note = Synthetic Construct
GSGGGS

SEQ ID NO: 227 - protein, 16 aa, synthetic construct
  REGION 1..16: note = Synthetic Construct
LLQGPLGLAGGSGGGS

SEQ ID NO: 228 - protein, 120 aa, synthetic construct [sequence omitted]

SEQ ID NO: 229 - protein, 111 aa, synthetic construct [sequence omitted]

SEQ ID NO: 230 - protein, 119 aa, synthetic construct
  REGION 1..119: note = Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSEDTAIYYCARALTYYDYEFAYWGQGTLVTVSA

SEQ ID NO: 231 - protein, 107 aa, synthetic construct
  REGION 1..107: note = Synthetic Construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 232 - protein, 125 aa, synthetic construct
  REGION 1..125: note = Synthetic Construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSS

SEQ ID NO: 233 - protein, 107 aa, synthetic construct
  REGION 1..107: note = Synthetic Construct
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIK

SEQ ID NO: 234 - protein, 120 aa, synthetic construct [sequence omitted]

SEQ ID NO: 235 - protein, 107 aa, synthetic construct [sequence omitted]

SEQ ID NO: 236 - protein, 118 aa, synthetic construct
  REGION 1..118: note = Synthetic Construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 237 - protein, 108 aa, synthetic construct
  REGION 1..108: note = Synthetic Construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 238 - protein, 117 aa, synthetic construct
  REGION 1..117: note = Synthetic Construct
EAKLQESGPVLVKPGASVKMSCKASGYTFTDYYMNWVKQSHGKSLEWIGVINPYNGDTSYNQKFKGKATLTVDKSSSTAYMELNSLTSEDSAVYYCARYYGSWFAYWGQGTLITVST

SEQ ID NO: 239 - protein, 112 aa, synthetic construct
  REGION 1..112: note = Synthetic Construct
DIVMTQTTLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 240 - protein, 7 aa, synthetic construct
  REGION 1..7: note = Synthetic Construct
KSRTTNG

SEQ ID NO: 241 - protein, 8 aa, synthetic construct
  REGION 1..8: note = Synthetic Construct
KGSRTTNG

(19) United States
(12) Patent Application Publication
(10) Pub. No.: US 2024/0327527 A1
(43) Pub. Date: Oct. 3, 2024

(54) ACTIVATABLE ANTIBODIES AND METHODS OF USE THEREOF

(71) Applicant: ALX Oncology Inc., South San Francisco, CA (US)

(72) Inventors: Marija VRLJIC, San Mateo, CA (US); Jaume PONS, San Francisco, CA (US)

(21) Appl. No.: 18/436,554

(22) Filed: Feb. 8, 2024

Related U.S. Application Data
(63) Continuation of application No. 16/613,774, filed on Nov. 14, 2019, now Pat. No. 11,939,385.
(60) Provisional application No. 62/507,129, filed on May 16, 2017.

(51) Int. Cl. C07K 16/28; A61K 39/00; A61P 35/00; C07K 14/47; C07K 14/52; C07K 14/54; C07K 16/32

(52) U.S. Cl. CPC C07K 16/2863; A61P 35/00; C07K 14/47; C07K 14/52; C07K 14/5428; C07K 14/5434; C07K 16/2818; C07K 16/32; A61K 2039/505; C07K 2317/31; C07K 2317/41; C07K 2317/55; C07K 2317/62; C07K 2317/76; C07K 2319/01; C07K 2319/30; C07K 2319/50

(57) ABSTRACT

The present application provides activatable antibodies comprising an antibody comprising an antigen-binding domain (ABD), wherein the ABD comprises a heavy chain variable region (VH) and a light chain variable region (VL), wherein the N-terminus of the VH is fused to a first polypeptide shield moiety (S1), and the N-terminus of the VL is fused to a second polypeptide shield moiety (S2), wherein S1 comprises a first disease-sensing releasable moiety (DS1) and/or S2 comprises a second disease-sensing releasable moiety (DS2), wherein association of S1 with S2 blocks binding of the ABD to its target, and wherein the ABD does not specifically bind to S1, S2, or association thereof. Composition, methods of treatment using the activatable antibodies, and methods of preparation thereof are further provided.

### CROSS-REFERENCE TO RELATED APPLICATION

This application claims priority to U.S. Provisional Application No. 62/507,129, filed on May 16, 2017, the contents of which are hereby incorporated by reference in their entirety.

### SUBMISSION OF SEQUENCE LISTING ON ASCII TEXT FILE

The content of die following submission on ASCII text file is incorporated herein by reference in its entirety: a computer readable form (CRF) of the Sequence Listing (tile name: 781282000140SEQLIST.txt, date recorded: May 15, 2018, size: 158 KB).

### FIELD OF THE INVENTION

The present invention relates to activatable antibodies and methods of use thereof.

### BACKGROUND OF THE INVENTION

Antibodies have successfully been used as therapeutic and diagnostic agents for a variety of diseases. A native antibody acts by binding to a specific target. However, antibody targets can be expressed in both diseased (e.g. tumor) tissues and normal tissues. When antibodies bind to targets expressed in normal tissues of a patient, the patient can experience undesirable, toxic side-effects, which can range from mild to severe or even life-threatening degree of intensity. Such side effects can lead to a decreased effective dosage of the antibody, which can lower its therapeutic efficacy. The antibody treatment may even be discontinued due to the side effects, which, in some cases, renders the antibody therapeutic unavailable to human patients. Thus, there exists an unmet need to engineer antibodies that preferentially bind to targets on diseased cells and tissues.

### BRIEF SUMMARY OF THE INVENTION

The present application provides activatable antibodies, methods of preparing the activatable antibodies, and method of treating a disease in an individual using the activatable antibodies.

In one aspect of the present application, there is provided an activatable antibody comprising an antibody comprising an antigen-binding domain (ABD), wherein the ABD comprises a heavy chain variable region (VH) and a light chain variable region (VL), wherein the N-terminus of the VH is fused to a first polypeptide shield moiety (S1), and the N-terminus of the VL is fused to a second polypeptide shield moiety (S2), wherein S1 comprises a first disease-sensing releasable moiety (DS1) and/or S2 comprises a second disease-sensing releasable moiety (DS2), wherein association of S1 with S2 blocks binding of the ABD to its target, and wherein the ABD does not specifically bind to S1. S2, or association thereof. In some embodiments, the activatable antibody comprises a first polypeptide comprising, from N-terminus to C-terminus, S1-VH; and a second polypeptide comprising, from N-terminus to C-terminus. S2-VL, wherein S1 and S2 are associated covalently or non-covalently.

In some embodiments, S1 comprises DS1 and S2 does not comprise DS2. In some embodiments, S2 comprises DS2 and S1 does not comprise DS1. In some embodiments, S1 comprises DS1, and S2 comprises DS2. In some embodiments, DS1 is identical to DS2. In some embodiments, DS1 is different from DS2.

In some embodiments according to any one of the activatable antibodies described above, the association between S1 and S2 spans a length greater than about 35 Å. In some embodiments, S1 and/or S2 comprise at least 7 amino acid residues (such as at least 10 amino acid residues). In some embodiments, each of S1 and S2 is a polypeptide no more than about 58 amino acids long. In some embodiments, S1 and/or S2 are oligopeptides.

In some embodiments according to any one of the activatable antibodies described above, the association between S1 and S2 is covalent. In some embodiments, S1 comprises a first association moiety (B), and S2 comprises a second association moiety (C), and wherein B and C are conjugated to each other via a covalent bond. In some embodiments, the association between S1 and S2 is non-covalent. In some embodiments, S1 comprises a first association moiety (B), and S2 comprises a second association moiety (C), and wherein B and C interact with each other non-covalently.

In some embodiments according to any one of the activatable antibodies described above, wherein association between S1 and S2 is covalent, B and C are conjugated to each other via an isopeptide bond. In some embodiments, the isopeptide bond is formed between an acyl donor and an acyl acceptor by a transglutaminase. In some embodiments, B comprises the amino acid sequence of SEQ ID NO: 1, and C comprises the amino acid sequence of SEQ ID NO: 2. In some embodiments, B comprises the amino acid sequence of SEQ ID NO: 2, and C comprises the amino acid sequence of SEQ ID NO. 1.

In some embodiments according to any one of the activatable antibodies described above, wherein the association between S1 and S2 is covalent. B and C are cysteines, and wherein B and C are conjugated to each other via a disulfide bond. In some embodiments, each of B and C comprises the amino acid sequence of SEQ ID NO: 3.

In some embodiments according to any one of the activatable antibodies described above, wherein the association between S1 and S2 is covalent. B is a first unnatural amino acid residue, and C is a second unnatural amino acid residue. In some embodiments, B and C are selenocysteines, and wherein B and C are conjugated to each other via a diselenide bond.

In some embodiments according to any one of the activatable antibodies described above, wherein the association between S1 and S2 is non-covalent, B and C are two complementary halves of a split-protein. In some embodiments, the split-protein is selected from the group consisting of GFP. Ubiquitin, CnaB2, interleukins, and chemokines. In some embodiments, wherein: (a) one of B and C comprises the amino acid sequence of SEQ ID NO: 4 and the other comprises the amino acid sequence of SEQ ID NO: 5; or (b) one of B and C comprises the amino acid sequence of SEQ ID NO: 6 and the other comprises the amino acid sequence of SEQ ID NO: 7. In some embodiments, B and C associate to form a therapeutic protein.

In some embodiments according to any one of the activatable antibodies described above, wherein the association between S1 and S2 is non-covalent. B and C are monomeric subunits of a dimeric protein. In some embodiments, the dimeric protein is selected from the group consisting of HIV protease, relaxin-2, Myo10, STIL, Troponin C, defensin-5, IL-10. DHBN domain of human BLM helicase, Insulin, human copper chaperone, KIX domain of human RECQL5, galectin-1. TNF-alpha, human neutrophil peptide 1, CXCL5. CXCL4. IL TI-immunoglobulin-like transcript, and IL-12. In some embodiments, the dimeric protein is a homodimer. In some embodiments, each of B and C comprises an amino acid sequence selected from the group consisting of SEQ ID NOs: 8-10 In some embodiments, the dimeric protein is a heterodimer. In some embodiments, one of B and C comprises the amino acid sequence of SEQ ID NO. 11 and the other comprises the amino acid sequence of SEQ ID NO: 12. In some embodiments, B and C associate to form a therapeutic protein.

In some embodiments according to any one of the activatable antibodies described above, wherein the association between S1 and S2 is non-covalent. B and C are complementary binding peptides. In some embodiments. B and C associate to form a parallel coiled-coil homodimer. In some embodiments, each of B and C comprises an amino acid sequence selected from the group consisting of SEQ ID NO: 13-15. In some embodiments, B and C associate to form an anti-parallel coiled-coil heterodimer. In some embodiments, one of B and C comprises the amino acid sequence of SEQ ID NO: 16 and the other comprises the amino acid sequence of SEQ ID NO: 17. In some embodiments, B and C associate to form a therapeutic protein.

In some embodiments according to any one of the activatable antibodies described above, wherein the association between S1 and S2 is non-covalent, one of B and C is a second VH, and the other is a second VL, and wherein B and C associate to form an Fv. In some embodiments, each of B and C comprises a cysteine, and wherein B is further conjugated to C via a disulfide bond. In some embodiments, B and C associate to form a therapeutic protein.

In some embodiments according to any one of the activatable antibodies described above, wherein the association between S1 and S2 is non-covalent, one of B and C is a glycan linked to a glycosylation peptide sequence, and the other is a glycan-binding protein (such as lectin, ficolin, or ERGIC-53). In some embodiments, the glycosylation peptide sequence comprises the amino acid sequence of SEQ ID NO: 18. In some embodiments, the glycan-binding protein is M-ficolin.

In some embodiments according to any one of the activatable antibodies described above, DS1 and/or DS2 comprises a peptide substrate specific for an enzyme that is expressed at a diseased site. In some embodiments, DS1 and/or DS2 is a tumor-sensing releasable moiety. In some embodiments, DS1 and/or DS2 comprises a peptide substrate cleavable by a tumor-specific protease. In some embodiments, the tumor-specific protease is selected from the group consisting of matriptase, legumain, uPA, matrix metalloproteases, MMP1, MMP2, MMP9, gelatinase, PSA, human neutrophil elastase, proteinase 3, pro-urokinase. Factor Xa, cathepsin K and cathepsin B. In some embodiments, the DS1 and/or DS2 comprises the amino acid sequence of SEQ ID NO: 19, 172, 240 or 241. In some embodiments. DS1 and/or DS2 comprises at least two peptide substrates each specific for an enzyme that is expressed at a diseased site. In some embodiments. DS1 and/or DS2 comprises a pH-sensitive protein or peptide, e.g., a protein or peptide that undergoes conformational change at acidic pH.

In some embodiments according to any one of the activatable antibodies described above, DS1 and/or DS2 comprises a flexible peptide linker.

In some embodiments according to any one of the activatable antibodies described above, the antibody comprises two or more antigen-binding domains. In some embodiments, the two or more antigen-binding domains bind to the same target. In some embodiments, the two or more antigen-binding domains bind to different targets. In some embodiments, the antibody is a bispecific antibody. In some embodiments, the antibody comprises a heavy chain constant domain fused to the C-terminus of VH, and a light chain constant domain fused to the C-terminus of VL. In some embodiments, the antibody is a Fab or a (Fab)₂. In some embodiments, the antibody comprises an immunoglobulin Fc region. In some embodiments, the antibody is a Full length antibody. In some embodiments, the antibody is an antibody-drug conjugate (ADC).

In some embodiments according to any one of the activatable antibodies described above, the ABD specifically binds to a target selected from the molecules shown in Table 8, such as EGFR, CTLA-4, PD-1, CD-71 or PD-L1. In some embodiments, the ABD is derived from a therapeutic antibody selected from die antibodies shown in Table 9, such as Cetuximab, Trastuzumab, Tremelimumab, Ipilimumab. Nivolumab, Pembrolizumab, or Atezolizumab.

In some embodiments according to any one of the activatable antibodies described above, disruption (such as cleavage or conformational change) of DS1 and/or DS2 unblocks the binding of the ABD to its target.

In some embodiments, there is provided a vector for expressing any one of the activatable antibodies described above, comprising: (a) a first nucleic acid encoding a heavy chain comprising S1 fused to die N-terminus of the VH; and (b) a second nucleic acid encoding a light chain comprising S2 fused to the N-terminus of the VL. In some embodiments, there is provided a host cell comprising the vector.

In some embodiments, there is provided a method of preparing an activatable antibody, comprising: (a) culturing the host cell described under a condition that produces a fusion antibody comprising the heavy chain and the light chain; and (b) isolating the fusion antibody, thereby providing the activatable antibody. In some embodiments, the method further comprises treating the fusion antibody with a reducing agent followed by an oxidation agent. In some embodiments, the method further comprises treating the fusion antibody with a transglutaminase. In some embodiments, the method further comprises treating the fusion antibody with a glycosylase. In some embodiments, there is provided a composition comprising the fusion antibody prepared in step (b) of the any one of methods described above.

Also provided is a pharmaceutical composition comprising any one of the activatable antibodies described above and a pharmaceutically acceptable carrier.

Another aspect of the present application provides a method of treating a disease in an individual, comprising administering to the individual an effective amount of any one of the pharmaceutical compositions described above, wherein DS1 and/or DS2 are disrupted at a diseased site, thereby unblocking binding of the ABD to its target at the diseased site. In some embodiments, the disease is a cancer. In some embodiments, the cancer is selected from the group consisting of bladder cancer, brain cancer, breast cancer, cervical cancer, colorectal cancer, esophageal cancer, kidney cancer, leukemia, liver cancer, lung cancer, melanoma, non-Hodgkin's lymphoma, ovarian cancer, pancreatic cancer, prostate cancer, skin cancer, thyroid cancer and uterine cancer.

These and other aspects and advantages of the present invention will become apparent from the subsequent detailed description and the appended claims. It is to be understood that one, some, or all of the properties of the various embodiments described herein may be combined to form other embodiments of the present invention.

All references cited herein, including patent applications, patent publications, and Genbank Accession numbers are herein incorporated by reference, as if each individual reference were specifically and individually indicated to be incorporated by reference in its entirety.

### BRIEF DESCRIPTION OF THE DRAWINGS

FIG. 1A is a schematic diagram illustrating generation of exemplary activatable antibodies. Each activatable antibody has a steric shield covering its target-binding site(s).

FIG. 1B is a schematic diagram illustrating behavior of an exemplary activatable antibody in normal and diseased tissues.

FIG. 2A shows covalent interactions that can be used to generate steric shields in exemplary activatable antibodies, including transglutaminase-catalyzed isopeptide bond formation and Cys-Cys disulfide bridge formation.

FIG. 2B shows non-covalent interactions between two half fragments of a split-protein that generate a steric shield in an exemplary activatable antibody.

FIG. 2C shows non-covalent interactions between two monomeric subunits of a protein dimer that generate a steric shield in an exemplary activatable antibody.

FIG. 2D shows non-covalent interactions between two peptides binding to one another, which generate a steric shield in an exemplary activatable antibody.

FIG. 2E shows non-covalent interactions between a glycan-binding protein (such as lectin) binding to a glycan in a glycosylated polypeptide shield moiety, which generate a steric shield in an exemplary activatable antibody.

FIG. 3A shows positions of the heavy chain and light chain N-termini relative to the antigen binding site in the crystal structure of a Fab of Pembrolizumab (PDBID: 5JXE).

FIG. 3B shows a structural model of an exemplary steric shield based on the crystal structure of a Fab of Pembrolizumab (PDBID: 5JXE) A structural model of the peptide SCLSGRSDNHGG (SEQ ID NO: 145) was added to the native N-termini of both the heavy and light chains, and the peptides were joined by modeling Cys-Cys disulfide bridge formation.

FIG. 3C shows a structural model of an exemplary steric shield based on the crystal structure of a Fab of Cetuximab (PDBID: 1YY9). A structural model of the peptide LLQGLSGRSDNH (SEQ ID NO: 140) was added to the native N-terminus of the heavy chain, a structural model of the peptide GKGLSGRSDNH (SEQ ID NO. 141) was added to the native N-terminus of the light chain, and the peptides were joined b modeling transglutaminase-mediated crosslinking.

FIG. 3D shows a structural model of an exemplary steric shield based on the crystal structure of a diabody of Hu5F9 (PDBID: 51WL). A structural model of the peptide LLQGLSGRSDNH (SEQ ID NO: 140) was added to the native N-terminus of the heavy chain, and a structural model of the peptide GKGLSGRSDNH (SEQ ID NO: 141) was added to the native N-terminus of the light chain, and the peptides were joined by modeling transglutaminase-mediated crosslinking.

FIG. 3E shows a structural model of an exemplary steric shield based on the crystal structure of Trastuzumab (PDBID: 1N8Z) A structural model of the peptide LLQGLSGRSDNH (SEQ ID NO: 140) was added to the native N-terminus of the heavy chain, and a structural model of the peptide GKGLSGRSDNH (SEQ ID NO: 141) was added to the native N-terminus of the light chain, and the peptides were joined by modeling transglutaminase-mediated crosslinking.

FIG. 4 shows an SDS-PAGE gel demonstrating transglutaminase-mediated steric shield formation in two exemplary activatable antibodies (activatable EGFR Ab 1 and 2) under different reaction conditions.

FIG. 5 shows an SDS-PAGE gel demonstrating removal of steric shields via cleavage of the disease-sensing releasable moieties by the protease matriptase from two exemplary antibodies (activatable EGFR Ab 1 and 2) under different reaction conditions.

FIG. 6 shows an SDS-PAGE gel demonstrating steric shield formation via a Cys-Cys disulfide bridge during antibody expression in mammalian cells in two exemplary activatable antibodies (activatable EGFR Ab 3 and 4).

FIG. 7 shows an SDS-PAGE gel demonstrating steric shield formation via a Cys-Cys disulfide bridge by reduction an oxidation of Cys under different conditions, in two exemplary activatable antibodies (activatable EGFR Ab 3 and 4).

FIG. 8 shows an SDS-PAGE gel demonstrating removal of the steric shields via cleavage of the disease-sensing releasable moieties by the protease matriptase or uPA from two exemplary antibodies (activatable EGFR Ab 3 and 4) under different reaction conditions.

FIG. 9A shows a bar graph of surface plasmon resonance (SPR) data illustrating EGFR binding to an exemplary activatable anti-EGFR antibody (activatable EGFR Ab 2). The data demonstrated that the steric shields formed via transglutaminase catalysis in the activatable antibody substantially reduced its EGFR binding activity, and removal of the steric shields from the activatable antibody restored its EGFR binding activity.

FIG. 9B shows a bar graph of SPR data illustrating EGFR binding to two exemplary activatable anti-EGFR antibodies (activatable EGFR Ab 3 and 4) The data demonstrated that the steric shields formed by Cys-Cys disulfide bridges in the activatable antibodies substantially reduced their EGFR binding activities, and removal of the steric shields from the activatable antibodies restored their EGFR binding activities.

FIG. 10 shows classes of unnatural amino acids that can be incorporated as unnatural amino acid pairs or unnatural-natural amino acid pairs to serve as association moieties B and C, thereby allowing association between polypeptide shield moieties S1 and S2.

FIG. 11A shows an SDS-PAGE gel demonstrating transglutaminase-mediated steric shield formation on activatable EGFR Ab 1, and demonstrating shield release by cleavage of disease-sensing releasable moieties by protease matriptase. Activatable EGFR Ab 1 samples shown here were used for SPR studies shown in FIG. 11B.

FIG. 11B shows a bar graph of SPR data illustrating EGFR binding of activatable EGFR Ab 1. The data demonstrates that steric shield formation on activatable EGFR Ab 1 substantially reduces its EGFR binding activity and removal of the steric shields from the activatable antibody restores its EGFR binding activity.

FIG. 12A shows an SDS-PAGE gel demonstrating steric shield formation via a Cys-Cys disulfide bridge by reduction and oxidation of Cys under different conditions in exemplary activatable EGFR Ab 3, and demonstrating shield release by cleavage of disease-sensing releasable moieties by protease matriptase. Activatable EGFR Ab 3 samples shown here were used for SPR studies shown in FIG. 12B.

FIG. 12B shows SPR sensograms data illustrating EGFR binding of activatable EGFR Ab 3. The data demonstrates that steric shield formation on activatable EGFR Ab 3 substantially reduces its EGFR binding activity and that matriptase mediated shield release by cleavage of disease-sensing releasable moieties restores EGFR binding activity.

FIG. 12C shows a bar graph of SPR data illustrating EGFR binding of activatable EGFR Ab 3. The data demonstrates that steric shield formation on activatable EGFR Ab 3 substantially reduces its EGFR binding activity and that activation by matriptase mediated shield release by cleavage of disease-sensing releasable moieties restores EGFR binding activity.

FIG. 13A shows an SDS-PAGE gel demonstrating transglutaminase-mediated steric shield formation in an exemplary activatable Fab (activatable hHER-2 Fab) and shield release by cleavage of disease-sensing releasable moieties by protease matriptase. Activatable hHER-2 Fab samples shown here were used for SPR studies shown in FIGS. 13B-130.

FIG. 13B shows SPR sensograms illustrating HER-2 binding of an activatable hHER-2 Fab. Tbc data demonstrates that steric shield formation on activatable hHER-2 Fab substantially reduces its HER-2 binding activity and that activation by matriptase mediated shield release by cleavage of disease-sensing releasable moieties restores HER-2 binding activity.

FIG. 13C shows SPR sensograms shown in FIG. 13B fitted against simulated sensograms based on fractions of active Fab (Fab without shield) and inactive Fab (Fab with shield) present in the SPR experiment. Simulated sensograms demonstrate that decrease in HER-2 binding observed in the SPR sensograms of the activatable hHER-2 Fab with shield can be attributed to the inactive activatable hHER-2 Fab (Fab with shield).

FIG. 13D shows a bar graph illustrating comparison between fractions of active activatable hHER-2 Fab (estimated from simulated sensograms of FIG. 13C) and fractions of activatable hHER-2 Fab without shield in place (estimated from the SDS-PAGE gel data of FIG. 13A). This data demonstrates a positive correlation between the fraction of activatable hHER-2 Fab without shield and the fraction of active hHER-2 Fab that binds HER-2.

FIG. 14A shows an SDS-PAGE gel demonstrating transglutaminase-mediated steric shield formation in an exemplary activatable Fab (activatable mCTLA-4 Fab) and shield release by cleavage of disease-sensing releasable moieties by proteases matriptase and MMP9. Activatable mCTLA-4 Fab samples shown here were used for SPR studies shown in FIGS. 14B-14D.

FIG. 14B shows SPR sensograms illustrating binding to mouse CTLA-4 of activatable mCTLA-4 Fab. The data demonstrates that steric shield formation on activatable mCTLA-4 Fab substantially reduces its CTAL4 binding activity and that matriptase mediated shield release by cleavage of disease-sensing releasable moieties restores mouse CTLA-4 binding.

FIG. 14C shows SPR sensograms shown in FIG. 14B fitted against simulated sensograms based on fractions of active Fab (Fab without shield) and inactive Fab (Fab with shield) present in the SPR experiment. Simulated sensograms demonstrate that decrease in mouse CTLA-4 binding observed in the SPR sensograms of activatable mCTLA-4 Fab with shield can be attributed to the inactive activatable mCTLA-4 Fab (Fab with shield).

FIG. 14D shows a bar graph illustrating comparison between the fractions of active activatable mCTLA-4 Fab that binds mouse CTLA-4 (estimated from the simulated sensograms of FIG. 14C) and the fractions of Fab without shield (estimated from SDS-PAGE gel data in FIG. 14A). This data demonstrates a positive correlation between the fraction of activatable mCTLA-4 Fab without shield and the fraction of active activatable mCTLA-4 Fab that binds mouse CTLA-4.

FIG. 15A shows in vitro cytotoxicity of activatable EGFR Ab 2 with shield or with shield released by protease matriptase against an EGFR-sensitive human lung carcinoma cell line H292. This data demonstrates that the presence of shield decreases binding of the activatable EGFR Ab 2 to EGFR on H292, and shield removal by matriptase increases binding to EGFR on 11292.

FIG. 15B shows m vitro cytotoxicity of activatable EGFR Ab 2 with shield or with shield released by protease matriptase against an EGFR-sensitive human colorectal adenocarcinoma cell line SW48. This data demonstrates that the presence of shield decreases binding of the activatable EGFR Ab 2 to EGFR on SW48, and shield removal by matriptase increases binding to EGFR on SW48.

FIG. 16A shows in vitro cytotoxicity of activatable EGFR Ab 3 with shield or with shield released by protease matriptase, or parent anti-EGFR antibody against an EGFR-sensitive human lung carcinoma cell line H292.

FIG. 16B shows in vitro cytotoxicity of activatable EGFR Ab 3 with shield or with shield released by protease matriptase, or parent anti-EGFR antibody against an EGFR-sensitive human colorectal adenocarcinoma cell line SW48.

FIG. 17A shows in vitro cytotoxicity of activatable EGFR Ab 4 with shield or with shield released by protease matriptase against an EGFR-sensitive human lung carcinoma cell line 1292.

FIG. 17B shows in vitro cytotoxicity of activatable EGFR Ab 4 with shield or parent anti-EGFR antibody against an EGFR-sensitive human colorectal adenocarcinoma cell line SW48.

### DETAILED DESCRIPTION OF THE INVENTION

The present application provides an activatable antibody having steric shield(s) that block the antigen binding site(s) of an antibody (including an antibody fragment) so that in the inactive state, the antibody is unable to bind to its antigen. The steric shield is generated by covalent or non-covalent association of two polypeptide shield moieties that extend from the N-termini of the heavy chain variable region (VH) and the light chain variable region (VL) of the antibody across an antigen binding site. One or both of the polypeptide shield moieties contain a disease-sensing releasable moiety that can be preferentially disrupted (such as cleaved or deformed) at a diseased site, resulting in release of the steric shield from the antigen binding site, and activation of the activatable antibody at the diseased site. In some embodiments, the steric shield itself is a therapeutic agent, which can act independently on its own target upon activation of the activatable antibody.

The design of the activatable antibodies described in the present application is applicable to any therapeutic antibodies. The activatable antibodies can be prepared by the following steps: the polypeptide shield moieties can be introduced into a therapeutic antibody by genetic engineering, and the steric shield can be formed during protein expression, or via a simple transglutaminase-catalyzed or oxidation/reduction step post protein expression. The activatable antibodies described herein can improve the pharmacokinetics and reduce unwanted side-effects of traditional therapeutic antibodies, and enable antibody therapy for previously unavailable targets due to excessive on-target off-tumor (or other diseased tissue) binding activities.

Accordingly, one aspect of the present application provides an activatable antibody comprising an antibody comprising an antigen-binding domain (ABD), wherein the ABD comprises a heavy chain variable region (VH) and a light chain variable region (VL), wherein the N-terminus of the VH is fused to a first polypeptide shield moiety (S1), and the N-terminus of the VL is fused to a second polypeptide shield moiety (S2), wherein S1 comprises a first disease-sensing releasable moiety (DS1) and/or S2 comprises a second disease-sensing releasable moiety (DS2), wherein association of S1 with S2 blocks binding of the ABD to its target, and wherein the ABD does not specifically bind to S1. S2, or association thereof. In some embodiments, each of S1 and S2 is a polypeptide no more than about 50 amino acids long. In some embodiments, S1 comprises a first association moiety (B), and S2 comprises a second association moiety (C), wherein B and C interact with each other non-covalently, and wherein B and C do not associate to form a second antigen-binding domain (e.g., an Fv).

Further provided are nucleic acids and vectors comprising a sequence encoding the activatable antibody described herein, host cells comprising the nucleic acids or vectors, and methods of producing the activatable antibodies. Further provided herein are methods of treating a disease using the activatable antibodies, and compositions (such as pharmaceutical compositions) comprising the activatable antibodies, medicine, kits, and articles of manufacture useful for the methods described herein.

### I. Definitions

Terms are used herein as generally used in the art, unless otherwise defined as follows.

The term “antibody” wherein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term antibody includes, but is not limited to, fragments that are capable of binding antigen, such as Fv. Fab, Fab′, and (Fab′)₂. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. The term antibody also includes, but is not limited to, chimeric antibodies, humanized antibodies, and antibodies of various species such as mouse, human, cynomolgus monkey, etc.

The term “monoclonal antibody” as used herein refers to an antibody obtained from a population of substantially homogeneous antibodies, i.e., the individual antibodies comprising the population are identical and/or bind die same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used n accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “hypervariable region” or “HVR.” as used herein, refers to each of the regions of an antibody variable domain which arm hypervariable in sequence. HVRs may form structurally defined loops (“hypervariable loops”). Generally, native four-chain antibodies comprise six HVRs: three in the VH (H1, H2, H3), and three in the VL (L1, L2, L3) HVRs generally comprise amino acid residues from the hypervariable loops and/or from the “complementarity determining regions” (CDRs). CDRs being of highest sequence variability and/or involved in antigen recognition. Exemplary hypervariable loops occur at amino acid residues 26-32 (L1), 50-52 (L2), 91-96 (13), 26-32 (H1), 53-55 (H2), and 96-101 (H3). (Chothia and Lesk, J. Mol. Biol 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, MD (1991)) With the exception of CDR1 in VH, CDRs generally comprise the amino acid residues that form the hypervariable loops. CDRs also comprise “specificity determining residues,” or “SDRs.” which are residues that contact antigen. SDRs are contained within regions of the CDRs called abbreviated-CDRs, or a-CDRs. Exemplary a-CDRs (a-CDR-1, a-CDR-1.2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front Biosci. 13:1619-1633 (2008)). Unless otherwise indicated, HVR residues and other residues in the variable domain (e.g., FR residues) are numbered herein according to Kabat e al., supra. Use of the term “or” herein is not meant to imply that alternatives are mutually exclusive.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four framework regions (FRs) and three hvpervariable regions (HVRs), arranged from amino-terminus to carboxy-terminus in the following order: FR1, HVR1, FR2, HVR2, FR3, HVR3, FR4. (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “heavy chain constant region” as used herein refers to a region comprising at least three heavy chain constant domains, CH1, CH2, and CH3, and a hinge region between CH1 and CH2. Non-limiting exemplary heavy chain constant regions include γ, δ, and α. Non-limiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a g constant region is an IgM antibody, and an antibody comprising an α constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a poly peptide comprising a heavy chain variable region and a heavy chain constant region, with or without a leader sequence.

The term “light chain constant region” as used herein refers to a region comprising a light chain constant domain. Cl. Non-limiting exemplary light chain constant regions include λ and κ.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

“Affinity” refers to the strength of the sum total of noncovalent interactions between a single binding site of a molecule (e.g., an antibody) and its binding partner (e.g., an antigen). The affinity of a molecule X for its partner Y can generally be represented by the dissociation constant (K_{d}). Affinity can be measured by common methods known in the art, including those described herein.

The term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In some embodiments, the extent of binding of an antibody to an unrelated target is less than about 10% of the binding of the antibody to the target as measured, e.g., by a radioimmunoassay (RIA). In some embodiments, an antibody that specifically binds to a target has a dissociation constant (Kd) of: ≤1 μM, ≤100 nM, ≤10 nM, ≤1 nM, or ≤0.1 nM. In some embodiments, an antibody specifically binds to an epitope on a protein that is conserved among the protein from different species. In another embodiment, specific binding can include, but does not require exclusive binding.

The term “multispecific” as used in conjunction with an antibody refers to an antibody having polyepitopic specificity (i.e., is capable of specifically binding to two, three, or more, different epitopes on one biological molecule or is capable of specifically binding to epitopes on two, three, or more, different biological molecules).

An “affinity matured” antibody refers to an antibody with one or more alterations in one or more hypervariable regions (HVRs) compared to a parent antibody which does not possess such alterations, such alterations resulting in an improvement in the affinity of the antibody for antigen. In some examples, an affinity matured antibody refers to an antibody with one or more alterations in one or more complementarity determining regions (CDRs) compared to a parent antibody which does not possess such alterations, such alterations resulting in an improvement in the affinity of the antibody for antigen.

A “chimeric antibody” as used herein refers to an antibody in which a portion of the heavy and/or light chain is derived from a particular source or species, while the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an Fab, a (Fab′)₂, etc.

An “HVR-grafted antibody” as used herein refers to a humanized antibody in which one or more hvpervariable regions (HVRs) of a first (non-human) species have been grafted onto the framework regions (FRs) of a second (human) species. In some examples, a “CDR-grafted antibody” as used herein refers to a humanized antibody in which one or more complementarity determining regions (CDRs) of a first (non-human) species have been grafted onto the framework regions (FRs) of a second (human) species.

A “human antibody” as used herein refers to antibodies produced in humans, antibodies produced in non-human animals that comprise human immunoglobulin genes, such as XENOMOUSE®, and antibodies selected using in vitro methods, such as phage display, wherein the antibody repertoire is based on a human immunoglobulin sequence.

The terms “nucleic acid molecule”. “nucleic acid” and “polynucleotide” may be used interchangeably, and refer to a polymer of nucleotides. Such polymers of nucleotides may contain natural and/or unnatural nucleotides, and include, but are not limited to, DNA, RNA, and PNA. “Nucleic acid sequence” refers to the linear sequence of nucleotides that comprise the nucleic acid molecule or polynucleotide.

The terms “polypeptide” and “peptide” are used interchangeably to refer to a polymer of amino acid residues, and are not limited to a minimum length. Such polymers of amino acid residues may contain natural or unnatural amino acid residues. Both full-length proteins and fragments thereof are encompassed by the definition. The terms also include post-expression modifications of the polypeptide, for example, glycosylation, sialylation, acetylation, phosphorylation, and the like. Furthermore, a “polypeptide” includes modifications, such as deletions, additions, and substitutions (generally conservative in nature), to the native sequence, as long as the polypeptide maintains the desired activity. These modifications may be deliberate, as through site-directed mutagenesis, or may be accidental, such as through mutations of hosts which produce the proteins or errors due to PCR amplification.

The term “fusion” refers to genetically joining two polypeptide fragments to provide a single continuous polypeptide (“fusion polypeptide”). The two polypeptide fragments may be directly joined to each other, or joined via another polypeptide disposed therebetween. Routine recombinant DNA techniques or chemical gene synthesis can be used to provide nucleic acids that genetically encode a fusion polypeptide.

The term “conjugation” refers to chemically joining two chemical groups or moieties together via one or more covalent bonds formed therebetween.

The term “leader sequence” refers to a sequence of amino acid residues located at the N-terminus of a polypeptide that facilitates secretion of a polypeptide from a mammalian cell. A leader sequence may be cleaved upon export of the polypeptide from the mammalian cell, forming a mature protein. Leader sequences may be natural or synthetic, and they may be heterologous or homologous to the protein to which they are attached.

A “native sequence” polypeptide comprises a polypeptide having the same amino acid sequence as a polypeptide found in nature. Thus, a native sequence polypeptide can have the amino acid sequence of naturally occurring polypeptide from any mammal. Such native sequence polypeptide can be isolated from nature or can be produced by recombinant or synthetic means. The term “native sequence” polypeptide specifically encompasses naturally occurring truncated or secreted forms of the polypeptide (e.g., an extracellular domain sequence), naturally occurring variant forms (e.g., alternatively spliced forms) and naturally occurring allelic variants of the polypeptide.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein. “Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2. ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, e.g., retained/improved antigen binding, decreased immunogenicity, or improved ADCC or CDC.

TABLE 1
Exemplary Amino Acid Substitutions.

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr. Asn. Gin;
(3) acidic: Asp, Glu;
(4) basic: His, Lys, Arg;
(5) residues that influence chain orientation: Gly, Pro;
(6) aromatic: Trp, Tyr, Phe.
   Non-conservative substitutions will entail exchanging a member of one of these classes for another class.

The term “vector” is used to describe a polynucleotide that may be engineered to contain a cloned polynucleotide or polynucleotides that may be propagated in a host cell. A vector may include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that may be used in colorimetric assays, e.g., β-galactosidase) The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells, and insect cells. Non-limiting exemplary mammalian cells include, but are not limited to, NSO cells. PER.C6® cells (Crucell), and 293 and CHO cells, and their derivatives, such as 293-6E and DG44 cells, respectively. The term “cell” includes the primary subject cell and its progeny.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the poly peptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated”.

The terms “individual” or “subject” are used interchangeably herein to refer to an animal; for example a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder.

As used herein, “treatment” is an approach for obtaining beneficial or desired clinical results. “Treatment” as used herein, covers any administration or application of a therapeutic for disease in a mammal, including a human. For purposes of this invention, beneficial or desired clinical results include, but are not limited to, any one or more of: alleviation of one or more symptoms, diminishment of extent of disease, preventing or delaying spread (e.g., metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods of the invention contemplate any one or more of these aspects of treatment.

In the context of cancer, the term “treating” includes any or all of: inhibiting growth of cancer cells, inhibiting replication of cancer cells, lessening of overall tumor burden and ameliorating one or more symptoms associated with the disease.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

An “effective amount” refers to an amount of an agent or drug effective to treat a disease or disorder in a subject. In the case of cancer, the effective amount of the agent may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

The ten “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker described herein. In some embodiments, the manufacture or kit is promoted, distributed, or sold as a unit for performing the methods described herein.

It is understood that embodiments of the invention described herein include “consisting” and/or “consisting essentially of” embodiments.

Reference to “about” a value or parameter herein includes (and describes) variations that are directed to that value or parameter per sc. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### II. Activatable Antibodies

The present application provides activatable antibodies comprising one or more antigen-binding domains having their antigen-binding sites reversibly blocked by a steric shield. As used herein, “steric shield” refers to the covalent and/or non-covalent association between two polypeptide shield moieties (e.g., S1 and S2) that are fused to the N-termini of the heavy chain variable region (VH) and the light chain variable region (VL) across an antigen-binding site in the activatable antibody. One or each of the polypeptide shield moieties comprises a disease-sensing releasable moiety (e.g., DS1 or DS2), which is disrupted at a diseased site to release the steric block from the antigen-binding site. Thus, the switch between the inactive and active state of activatable antibody is provided by the presence of the disease-sensing releasable moiety. For example, in some embodiments, the disease-sensing releasable moiety is a tumor-sensing releasable moiety that can sense the presence of a tumor-specific reagent, such as tumor-specific protease or acidic pH, wherein in the absence of the tumor-specific reagent, the tumor-sensing releasable moiety stays intact, but in the presence of the tumor-specific reagent, the tumor-sensing releasable moiety falls apart, thus causing separation of the steric shield from the antigen binding site. The term “activatable” refers to antibody that can exist in two states: “inactive” state where target binding is reduced due to presence of associated shield moieties S1 and S2, and “active” state where target binding is increased due to disruption (e.g. cleavage) of associated S1 and S2 shield moieties. Experimentally measured “affinity” of activatable antibody and its target depends on relative fractions in a composition of “inactive” and “active” states present. As the fraction of “active” state increases measured “affinity” increases, and conversely, as the fraction of the “inactive” state increases, measured affinity decreases. Ratio of the “active” and “inactive” states is controlled by the agents (e.g. proteases) that disrupt (e.g. cleave) disease-sensing moieties DS1 and DS2. The affinity of an activatable antibody composition after disruption of associated S1 and/or S2 shield moieties may be about 5×, 10×, 20×, 30×, 40×, 50×, 60×, 70×, 80×, 90×, 100×, 200×, 500×, or 1000× higher than the affinity of the activatable antibody composition before disruption of associated S1 and/or S2 shield moieties. Affinities may be measured in vitro using target proteins or cells expressing target proteins.

In some embodiments, the activatable antibody is used as a pro-drug for specific delivery of a therapeutic antibody to a diseased site. Such activatable antibody has preferential activities at a diseased site (e.g. at a tumor site) than at non-diseased sites, wherein the therapeutic antibody can only bind to its target at the diseased site in the absence of the steric shield, and not to the same target in normal tissues in the presence of the steric shield. In some embodiments, the steric shield is itself a therapeutic agent such that upon activation of the activatable antibody in the diseased site, both the therapeutic antibody and the released steric shield become active therapeutic agents that can act independently or synergistically on targets in the diseased site. The present application further provides methods for generating a steric shield that blocks one or more binding site in a therapeutic antibody, thereby providing a pro-drug of the therapeutic antibody.

A native antibody comprises two polypeptide chains, known as the heavy chain and the light chain. The N-termini of the heavy chain variable region (VH) and the light chain variable region (VL) are positioned in close proximity to each antigen binding site. Inventors of the present application discovered that connection of the N-termini of the VH and VL via various genetically encoded shield moieties described herein result in steric shielding of the antigen binding site, thereby providing a universal strategy for inactivating the antigen-binding site(s) in any antibody (including antigen binding fragments). Inventors further engineered disease-tissue specific sensor(s) in the steric shield connecting the two N-termini such that in the presence of a disease specific element that interacts with the sensor moiety, the steric shield falls apart, allowing the steric shield to diffuse out of the w ay of the antigen binding sites, thus restoring the antibody's ability to bind its target(s).

Thus, in some embodiments, there is provided an activatable antibody comprising an antibody comprising an antigen-binding domain (ABD), wherein the ABD comprises a heavy chain variable region (Vii) and a light chain variable region (VL), wherein the N-terminus of the VH is fused to a first polypeptide shield moiety (S1), and the N-terminus of the VL is fused to a second polypeptide shield moiety (S2), wherein S1 comprises a first disease-sensing releasable moiety (DS1) and S2 comprises a second disease-sensing releasable moiety (DS2), wherein association of S1 with S2 blocks binding of the ABD to its target, and wherein the ABD does not specifically bind to S1, S2, or association thereof. In some embodiments, S1 further comprises a first association moiety B, and S2 further comprises a second association moiety C, wherein B and C associate with each other covalently and/or non-covalently. In some embodiments, disruption (e.g., cleavage) of DS1 and/or DS2 unblocks the binding of the ABD to its target. In some embodiments, DS1 and/or DS2 comprise a peptide substrates specific for an enzyme (e.g., protease) that is expressed at a diseased site. In some embodiments, DS1 and/or DS2 comprises a pH-sensitive protein or peptide.

In some embodiments, there is provided an activatable antibody comprising an antibody comprising an antigen-binding domain (ABD), wherein the ABD comprises a heavy chain variable region (VH) and a light chain variable region (Vt.), wherein the N-terminus of the VH is fused to a first polypeptide shield moiety (S1), and the N-terminus of the VL is fused to a second polypeptide shield moiety (S2), wherein S1 comprises a disease-sensing releasable moiety (DS1), wherein association of S1 with S2 blocks binding of the ABD to its target, and wherein the ABD does not specifically bind to S1, S2, or association thereof. In some embodiments, S1 comprises a first association moiety B, and S2 comprises a second association moiety C, wherein B and C associate with each other covalently and/or non-covalently. In some embodiments, disruption (e.g., cleavage) of DS1 unblocks the binding of the ABD to its target. In some embodiments, DS1 comprises a peptide substrates specific for an enzyme (e.g., protease) that is expressed at a diseased site. In some embodiments, DS1 comprises a pH-sensitive protein or peptide.

In some embodiments, there is provided an activatable antibody comprising an antibody comprising an antigen-binding domain (ABD), wherein the ABD comprises a heavy chain variable region (VH) and a light chain variable region (VL), wherein the N-terminus of the VH is fused to a first polypeptide shield moiety (S1), and the N-terminus of the VL is fused to a second polypeptide shield moiety (S2), wherein S2 comprises a disease-sensing releasable moiety (DS2), wherein association of S1 with S2 blocks binding of the ABD to its target, and wherein the ABD does not specifically bind to S1, S2, or association thereof. In some embodiments. S1 comprises a first association moiety B, and S2 comprises a second association moiety C, wherein B and C associate with each other covalently and/or non-covalently. In some embodiments, disruption (e.g., cleavage) of DS2 unblocks the binding of the ABD to its target. In some embodiments, DS2 comprises a peptide substrates specific for an enzyme (e.g., protease) that is expressed at a diseased site. In some embodiments, DS2 comprises a pH-sensitive protein or peptide.

In some embodiments, there is provided a fusion antibody comprising an antibody comprising an antigen-binding domain (ABD), wherein the ABD comprises a heavy chain variable region (VH) and a light chain variable region (VL), wherein the N-terminus of the VH is fused to a first polypeptide shield moiety (S1), and the N-terminus of the VL is fused to a second polypeptide shield moiety (S2), wherein S1 comprises a first disease-sensing releasable moiety (DS1) and/or S2 comprises a second disease-sensing releasable moiety (DS2), wherein S1 is capable of associating with S2 but S1 is not associated with S2, wherein the association between S1 and S2 blocks binding of the ABD to its target, and wherein the ABD does not specifically bind to S1, S2, or association thereof.

In some embodiments, there is provided an activatable antibody comprising an antibody comprising an antigen-binding domain (ABD), wherein the ABD comprises a heavy chain variable region (VH) and a light chain variable region (VL), wherein the N-terminus of the VH is fused to a first polypeptide shield moiety (S1), and the N-terminus of the V L is fused to a second polypeptide shield moiety (S2), wherein each of S1 and S2 is a polypeptide no more than about 50 amino acids long, wherein S1 comprises a first disease-sensing releasable moiety (DS1) and/or S2 comprises a second disease-sensing releasable moiety (DS2), wherein association of S1 with S2 blocks binding of the ABD to its target, and wherein the ABD does not specifically bind to S1, S2, or association thereof.

In some embodiments, there is provided an activatable antibody comprising an antibody comprising an antigen-binding domain (ABD), wherein the ABD comprises a heavy chain variable region (VH) and a light chain variable region (VL), wherein the N-terminus of the VH is fused to a first polypeptide shield moiety (S1), and the N-terminus of the VL is fused to a second polypeptide shield moiety (S2), wherein S1 comprises a first disease-sensing releasable moiety (DS1) and/or S2 comprises a second disease-sensing releasable moiety (DS2), wherein S1 comprises a first association moiety (B), and S2 comprises a second association moiety (C), wherein B and C interact with each other non-covalently, wherein B and C do not associate to form a second antigen-binding domain (e.g., an Fv), and wherein association of S1 with S2 blocks binding of the ABD to its target, and wherein the ABD does not specifically bind to S1, S2, or association thereof.

In some embodiments, the activatable antibody binds with higher affinity to its antigen on diseased cells or at a diseased site than on non-diseased cells. In some embodiments, the activatable antibody binds to its antigen under conditions characteristic for the diseased site (e.g. at the site of or inside a tumor). In some embodiments, the activatable antibody binds with higher affinity in the presence of diseased-site specific proteases. In some embodiments, the activatable antibody binds to its target with higher affinity under acidic pH (e.g. less than around pH 7.0) than at normal pH. In some embodiments, the activatable antibody binds to its target with higher affinity under hypoxic conditions (e.g. compared to normal physiological conditions at non-diseased site).

S1 and S2 are polypeptides that can have the same sequences or different sequences. S1 and S2 can comprise any natural or unnatural amino acid residues, including post-translational modifications, such as glycosylation, phosphorylation, etc. In some embodiments, S1 and S2 are oligopeptides. In some embodiments, S1 and S2 are protein domains or fragments. In some embodiments, one of S1 and S2 is an oligopeptide, and the other is a protein. In some embodiments. S1 and/or S2 comprises at least about any one of 5, 10, 15, 20, 25, 30, 50, 100, 150, 200 or more amino acid residues. In some embodiments, S and/or S2 is an oligopeptide of about any one of 1, 1, 12, 13, 14, 15, 16, 17, 18, 19, 20, 21, 22, 23, 24, 25, 26, 27, 28, 29, 30 amino acids long. In some embodiments, S1 and/or S2 is an oligopeptide having no more than about any one of 50, 45, 40, 35, 30, 29, 28, 27, 26, 25, 24, 23, 22, 21, 20, 19, 18, 17, 16, 15, 14, 13, 12, 11, or no amino acids. In some embodiments, S1 and/or S2 is a protein, domain or fragment thereof no more than about any one of 300, 250, 200, 100, or 50 amino acids long.

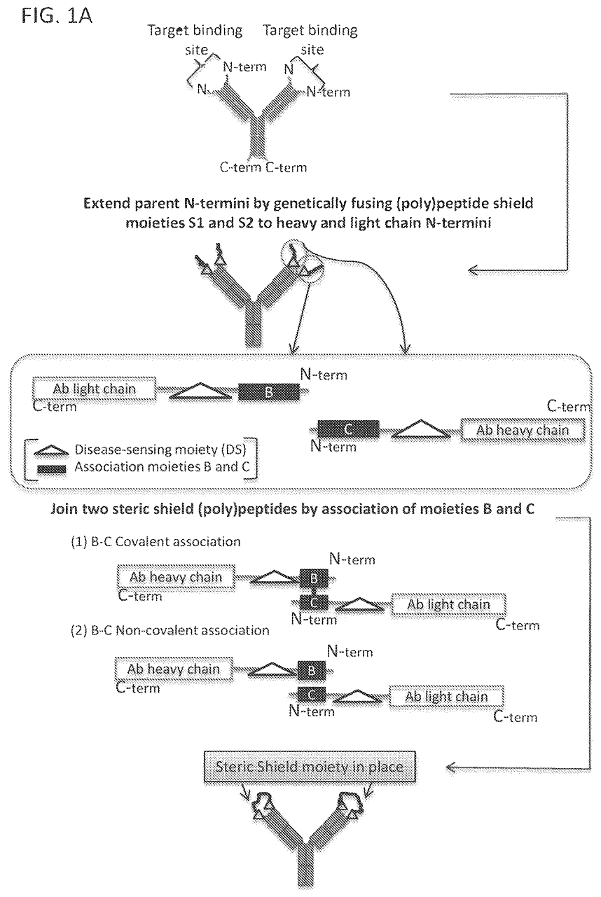

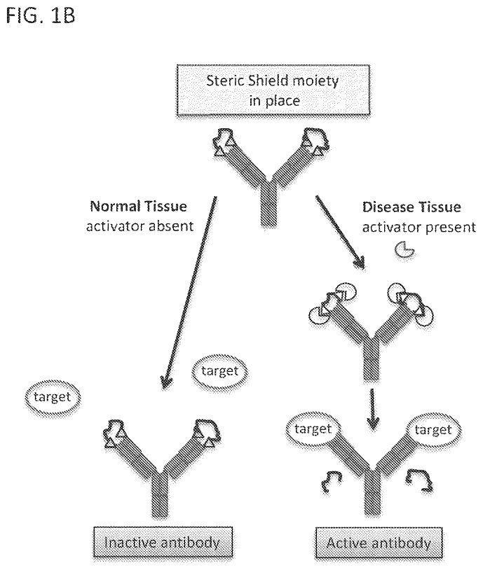

Upon association between B and C, the resulting S1-S2 steric shield is of sufficient length to span the distance between the N-termini of the VH and VL of the antibody component. This steric shield hoovers over the antigen binding site at a distance that prevents antigen binding until it is released via disruption of the disease-sensing releasable moieties DS1 and/or DS2 (e.g., FIG. 1B). In some embodiments, the association between S1 and S2 spans the entire length between the N-termini of the VH and VL. In some embodiments, the association between S1 and S2 spans a length greater than about any one of 35 Å, 40 Å, 45 Å, 50 Å, 55 Å, 60 Å, 65 Å, 70 Å, 75 Å, or 80 Å. In some embodiments, the association between S1 and S2 spans a length that is no more than about any one of 80 Å, 75 Å, 70 Å, 65 Å, 60 Å, 55 Å, 50 Å, 45 Å, 40 Å, or 35 Å.

As used herein, the “N-terminus” of a polypeptide refers to the first N-terminal amino acid of the polypeptide. In some embodiments, S1 is fused to the N-terminus of a native VH, and/or S2 is fused to the N-terminus of a native VL. In some embodiments, S1 is fused to the N-terminus of an engineered VH, and/or S2 is fused to the N-terminus of an engineered VL. In some embodiments, an engineered VH or VL has an amino acid variation in any one or more of the N-terminal residues 1-5 in the FR1 region of the VH or VL, including, but not limited to substitution, deletion, and/or insertion, which do no substantially reduce the binding affinity of VH, VL or the antigen-binding domain to its target antigen. For example, the binding affinity is reduced by less than any of 50%, 40%, 30%, 20%, and 10% as compared to the binding affinity of the antigen-binding domain without the amino acid variation.

Non-limiting exemplary schematics of the activatable antibody are shown in the formula below, wherein L₁, L₂, L₃, and L₄ are optional peptide linkers, and “-” between B and C indicate covalent and/or non-covalent interactions.

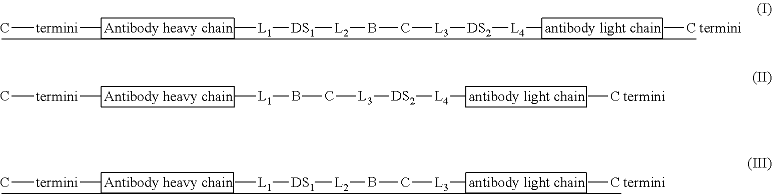

In some embodiments, B and C are residues or chemical groups as part of or attached to DS1, DS2, or the peptide linkers. In some embodiments, B is a polypeptide that extends from DS1, and C is a polypeptide extending from DS2. In some embodiments, one of B and C is a polypeptide extending from DS1 or DS2, and the other is a residue or chemical group as part of or attached to DS1. DS2 or the peptide linkers.

In some embodiments, B and C are complementary interaction moieties that are different from each other. In other embodiments. B and C are the same. In some embodiments, B and C are conjugated to each other by a covalent bond, such as a disulfide bond, diselenide bond, or isopeptide bond (e.g., via transglutamination). In some embodiments, B and C are protein domains or fragments, or complementary binding peptides that associate with each other non-covalently. In some embodiments, B and C associate with each other by both covalent and non-covalent interactions.

The various components of the activatable antibody, including modes of association between B and C (or S1 and S2), the disease-sensing releasable moieties (DS1 and DS2), peptide linkers, and the antibody component, are described in sections A-E below. One of skill in the art would readily appreciate that any one, some, or all properties described herein for each component can be combined in any suitable manner to form embodiments of the present invention.

### A. Covalent Association

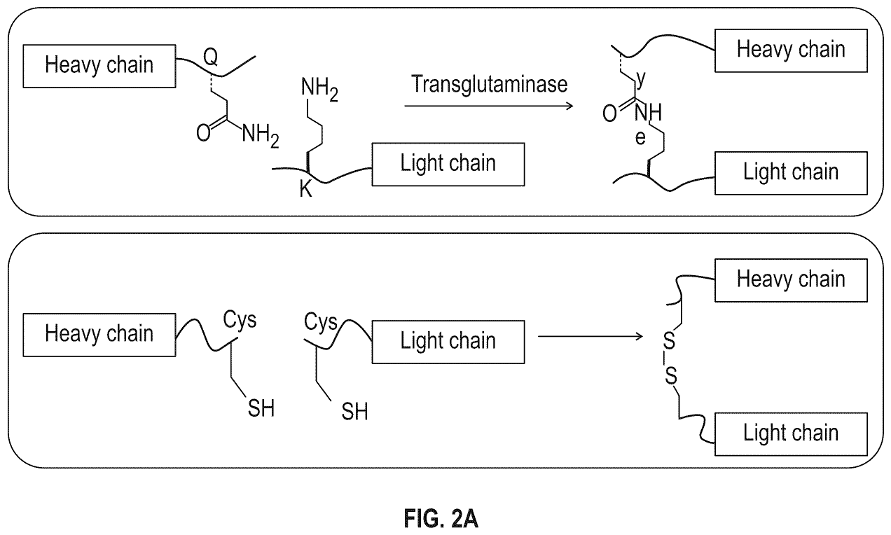

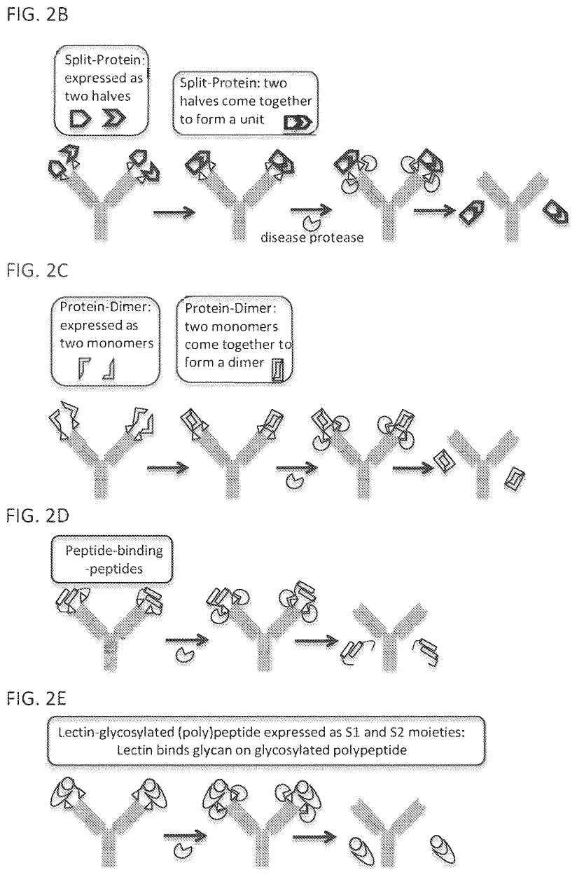

In some embodiments, the association between S1 and S2 is covalent. In some embodiments. S1 comprises a first association moiety (B), and S2 comprises a second association moiety (C), and wherein B and C are conjugated to each other via a covalent bond. See, for example. FIG. 2A. In some embodiments, B further interacts with C non-covalently, such as with any one of the non-covalent interactions described in Section B. B and C can be genetically encoded and engineered into the activatable antibodies. The conjugation between B and C can be formed during expression of the antibody, or by treating the expressed fusion protein comprising the heavy chain and light chain polypeptides of the activatable antibody with an enzyme (e.g., transglutaminase) or chemical reagents (such as reduction and oxidation reagents) to allow the formation (or re-formation) of one or more covalent bonds between B and C.

Non-limiting examples of B and C that allow covalent association between S1 and S2 are described in Sections 1-3 below.

### 1. Transglutamination

In some embodiments, B and C are conjugated to each other via an isopeptide bond. In some embodiments, B and C are conjugated to each other by a transglutamination reaction. In some embodiments, the transglutamination reaction is catalyzed by a transglutaminase. For example, see the schematics in FIG. 1A and FIG. 2A. Transglutaminases transfer the γ-glutaminyl of an acyl donor glutamine from a polypeptide to an acyl acceptor amine group, such as primary amine or the ε-amino group of lysine, resulting in an isopeptide bond connecting the acyl donor glutamine and the acyl acceptor residue. In some embodiments, the transglutaminase preferentially recognizes a peptide sequence that harbors an amino acid residue having an acyl acceptor group, and a peptide sequence that harbors an amino acid residue having an acyl donor group. The amino acid residue having the acyl acceptor group is referred herein as the “acyl acceptor residue” (e.g., lysing, N-terminal glycine) and the amino acid residue having the acyl donor group is referred herein as the “acyl donor residue” (e.g., glutamine). In some embodiments, B comprises an amino acid sequence comprising an acyl donor residue, and C comprises an amino acid sequence comprising an acyl acceptor residue and wherein B and C are conjugated to each other by a transglutaminase. In some embodiments, C comprises an amino acid sequence comprising an acyl acceptor residue, and B comprises an amino acid sequence comprising an acyl donor residue, and wherein B and C are conjugated to each other by a transglutaminase.

Exemplary acyl donor and acyl acceptor residue containing sequences are shown in Table 2.

TABLE 2
Exemplary acyl donor and acyl acceptor sequences.

| SEQ | | |
|---|---|---|
| ID | | |
| NO | Type | Sequence |
| 52 | Acyl | XXQX, wherein X is any natural or unnatural amino acid |
| 53 | donor | LLQX₁X₂X₃X₄X₅, wherein X₁ is G or P, wherein X₂ is A, G, |
| | | P, or absent, wherein X₃ is A, G, K, P, or absent, wherein X₄ |
| | | is G, K, or absent, and wherein X₅ is K or absent |
| 1 | | LLQG |
| 54 | | VLNLAQSKNFH (Human Interleukin-2) |
| 55 | | APALQPTQGAM (Filgrastim) |
| 56 | | IPKEQKYSF (Human growth hormone) |
| 57 | | MGGSPLAQSHGGS (Myoglobin) |
| 58 | | KETAAAKFERQHMDS (S-tag) |
| 59 | | TEYGLFQINNDS (a-lactalbumin) |
| 60 | | APQQEA (Human osteonectin) |
| 61 | | EDGFFKI (Human fibrillin-1) |
| 62 | | EEQYNSTYR (IgG) |
| 51 | Acyl | X₁KX₂, wherein X₁ and X₂ are any natural or unnatural |
| | acceptor | amino acids |
| 2 | | GKG |
| 74 | | GGG |
| 75 | | GGGG |

In some embodiments, the acyl donor residue is a glutamine (Q). In some embodiments, the sequence comprising the acyl donor residue is XXQX (SEQ ID NO: 52), wherein X is any natural or unnatural amino acid. In some embodiments, the sequence comprising the acyl donor residue is LLQX₁X₂X₃X₄X₅ (SEQ ID NO: 53), wherein X₁ is G or P, wherein X₂ is A, G, P, or absent, wherein X₃ is A, G, K, P, or absent, wherein X₄ is G, K, or absent, and wherein X₅ is K or absent. In some embodiments, the sequence comprising the acyl donor residue is LLQG (SEQ ID NO: 1). In some embodiments, the sequence comprising the acyl donor residue is any one of native transglutaminase acyl donor substrates found in naturally-occurring proteins, such as any one of SEQ ID NOs: 54-62. In some embodiments, the sequence comprising the acyl donor residue is any one of LQG (SEQ ID NO: 76), LLQGG (SEQ ID NO: 77), LSLSQG (SEQ ID NO: 78), GGGLLQGG (SEQ ID NO: 79), GLLQG (SEQ ID NO: 80), LLQ (SEQ ID NO: 81), GSPLAQSHGG (SEQ ID NO: 82), GLIQGGG (SEQ ID NO: 83), GLLQGG (SEQ ID NO: 84), GLLQ (SEQ ID NO: 85), LLQLLQGA (SEQ ID NO: 86), LLQGA (SEQ ID NO: 87), LLQYQGA (SEQ ID NO: 88), LLQGSG (SEQ ID NO: 89), LLQYQG (SEQ ID NO: 90), LLQLLQG (SEQ ID NO: 91), SLLQG (SEQ ID NO: 92), LLQLQ (SEQ ID NO: 93), LLQLLQ (SEQ ID NO: 94), LLQGR (SEQ ID NO: 95), LLQGPP (SEQ ID NO: 96), LLQGPA (SEQ ID NO: 97), GGLLQGPP (SEQ ID NO. 98), GGLLQGA (SEQ ID NO: 99), LLQGPGK (SEQ ID NO: 100), LLQGPG (SEQ ID NO: 101), LLQGP (SEQ ID NO: 102), LLQP (SEQ ID NO: 103), LLQPGK (SEQ ID NO: 104), LLQAPGK (SEQ ID NO: 105), LLQGAPG (SEQ ID NO: 106), LLQGAP (SEQ ID NO: 107), LLQLQG (SEQ ID NO: 108), LLQGA (SEQ ID NO: 109), LQG (SEQ ID NO: 110), GGLLQGA (SEQ ID NO: 111), LLQGPA (SEQ ID NO 112), LLQGPP (SEQ ID NO: 113), GGLLQGPP (SEQ ID NO: 114), LLQGWG (SEQ ID NO: 115), LLQG (SEQ ID NO: 116), LLQYQG (SEQ ID NO: 117), LLQLLQG (SEQ ID NO: 118), LLQLQG (SEQ ID NO. 119), LLQLLQ (SEQ ID NO: 120), LLQLQ (SEQ ID NO: 121), LLQGR (SEQ ID NO: 122), LLQYQGA (SEQ ID NO: 123), SLLQG (SEQ ID NO: 124), LLQLLQGA (SEQ ID NO: 125), LLQGPP (SEQ ID NO: 126), QVQLKE (SEQ ID NO: 127), VQLKE (SEQ ID NO: 128), GGLLQGG (SEQ ID NO: 129), TVQQEL (SEQ ID NO: I30), GQQQTPY (SEQ ID NO: 131), GLQQASV (SEQ ID NO: 132), WQTPMNS (SEQ ID NO: 133), WQHPLHDWFDLV (SEQ ID NO: 134), FQQPLDPWTSPI (SEQ ID NO: 135), HQSYVDPWMLDH (SEQ ID NO: 136), REQLYLDYNVFS (SEQ ID NO: 137), MWQKLPLVVHWPT (SEQ ID NO: 138), or THMYQSIYVPDI (SEQ ID NO: 139). See, for example, US20130230543A, US20130122020A1, WO2015/162563, US20150284713A1, US20140356385A1, Keresztessy Z et al. 2006 Protein Science 15: 2466-2480; and Sugimura Y et al. 2006 J Biol. Chem. 281.17699-17706; Biomaterials 31(2010) 4600-4608.

In some embodiments, the acyl acceptor residue is lysine (K). In some embodiments, the sequence comprising the acyl acceptor residue is X₂KX₂ (SEQ ID NO:51), wherein X₁ and X₂ are any natural or unnatural amino acids. In some embodiments, the sequence comprising the acyl acceptor residue is GKG (SEQ ID NO. 2). In some embodiments, the acyl acceptor residue is an N-terminal glycine. In some embodiments, the sequence comprising the acyl acceptor residue is GGG (SEQ ID NO: 74) or GGGG (SEQ ID NO:75). Also, see e.g., Tanaka T et al. 2005. FEBS Letters 579, 2092-2096.

In some embodiments, B comprises the amino acid sequence of SEQ ID NO:2 and C comprises the amino acid sequence of SEQ ID NO:1, or B comprises the amino acid sequence of SEQ ID NO:2 and C comprises the amino acid sequence of SEQ ID NO:1. In some embodiments, B comprises the amino acid sequence LLQGLSGRSDNH (SEQ ID NO:140) and C comprises the amino acid sequence GKGLSGRSDNH (SEQ ID NO:141), or B comprises the amino acid sequence of SEQ ID NO: 141 and C comprises the amino acid sequence of SEQ ID NO: 140. In some embodiments, S1 comprises the amino acid sequence LLQGLSGRSDNHGGGS (SEQ ID NO: 142) and S2 comprises the amino acid sequence GKGLSGRSDNHGGGS (SEQ ID NO: 143), or S1 comprises the amino acid sequence of SEQ ID NO: 143 and S2 comprises the amino acid sequence of SEQ ID NO:142. In some embodiments, S1 comprises the amino acid sequence LLQGPLGLAGGSGGGS (SEQ ID NO: 227) and S2 comprises the amino acid sequence GKGLSGRSDNHGGGS (SEQ ID NO. 143), or S1 comprises the amino acid sequence of SEQ ID NO:143 and S2 comprises the amino acid sequence of SEQ ID NO: 227.

In some embodiments, the activatable antibody comprises a first polypeptide comprising the amino acid sequence of SEQ ID NO: 21, and a second polypeptide comprising the amino acid sequence of SEQ ID NO: 22, wherein amino acid residue 3 (Q) in SEQ ID NO 21 is conjugated to amino acid residue 2 (K) of SEQ ID NO: 22 via isopeptide bond. In some embodiments, the activatable antibody comprises a first polypeptide comprising the amino acid sequence of SEQ ID NO: 23, and a second polypeptide comprising the amino acid sequence of SEQ ID NO: 24, wherein amino acid residue 3 (Q) in SEQ ID NO:23 is conjugated to amino acid residue 2 (K) of SEQ ID NO: 24 via isopeptide bond. In some embodiments, the activatable antibody comprises a first polypeptide comprising the amino acid sequence of SEQ ID NO: 29, and a second polypeptide comprising the amino acid sequence of SEQ ID NO: 30, wherein amino acid residue 3 (Q) in SEQ ID NO: 29 is conjugated to amino acid residue 2 (K) of SEQ ID NO: 30 via isopeptide bond. In some embodiments, the activatable antibody comprises a first polypeptide comprising the amino acid sequence of SEQ ID NO: 31, and a second polypeptide comprising the amino acid sequence of SEQ ID NO: 32, wherein amino acid residue 3 (Q) in SEQ ID NO: 31 is conjugated to amino acid residue 2 (K) of SEQ ID NO: 32 via isopeptide bond. In some embodiments, the activatable antibody comprises a first polypeptide comprising the amino acid sequence of SEQ ID NO: 33, and a second polypeptide comprising die amino acid sequence of SEQ ID NO: 34, wherein amino acid residue 3 (Q) in SEQ ID NO: 33 is conjugated to amino acid residue 2 (K) of SEQ ID NO: 34 via isopeptide bond. In some embodiments, the activatable antibody comprises a first polypeptide comprising the amino acid sequence of SEQ ID NO: 224, and a second polypeptide comprising the amino acid sequence of SEQ ID NO: 225, wherein amino acid residue 3 (Q) in SEQ ID NO: 224 is conjugated to amino acid residue 2 (K) of SEQ ID NO: 225 via isopeptide bond.

The transglutaminase used in the present application can be obtained or made from a variety of sources. See, e.g., US20130230543A1. In some embodiments, the transglutaminase is a naturally-occurring transglutaminase. In some embodiments, the transglutaminase is a calcium dependent transglutaminase which requires calcium to induce enzyme conformational changes and allow enzyme activity. For example, transglutaminase can be derived from guinea pig liver and obtained through commercial sources (e.g., Sigma-Aldrich (St Louis. Mo.) and MP Biomedicals (Irvine, Calif.)). In some embodiments, the transglutaminase is a calcium independent transglutaminase which does not require calcium to induce enzyme conformational changes and allow enzyme activity. In some embodiments, the transglutaminase is a microbial transglutaminase derived from a microbial genome, such as transglutaminase from Streptoverticillium or Streptomyces (e.g., Streptomyces mobarensis or Streptoverticillium mobarensis). Commercially available calcium independent transglutaminase such as ACTIVA™ (Ajinomoto, Japan) is suitable for the present invention. In some embodiments, the transglutaminase is a mammalian protein (e.g., human transglutaminase), a bacterial protein, a plant protein, a fungi protein (e.g., Oomycetes and Actinomycetes transglutaminases), or a prokaryotic protein. In some embodiments, the transglutaminase is from Micrococcus, Clostridium, Turolpsis, Rhizopus, Monascus, or Bacillus.

In some embodiments, the transglutaminase is a recombinant protein produced using recombinant techniques known to persons skilled in the art. In some embodiments, the transglutaminase is an engineered transglutaminase. In some embodiments, the transglutaminase is a purified protein. For example, the purified transglutaminase is least about 50% pure. In some embodiments, the purified transglutaminase is at least about any of 50%, 55%, 60%, 65%, 70%, 75%, 80%, 85%, 94%, 95%, 96%, 97%, 98%, 99%, or 100% pure.

### 2. Cys-Cys Disulfide Bond

In some embodiments, B and C are conjugated to each other by one or more (such as any of 1, 2, 3, or more) cysteine-cysteine disulfide bonds (see, e.g., the schematics in FIG. 1A and FIG. 2A). In some embodiments, B and C comprise an amino acid sequence comprising cysteine, and wherein B and C are conjugated to each other via a disulfide bond. In some embodiments, cysteines that form the disulfide bond are within DS1 and DS2 or peptide linkers in S1 and S2 respectively. In some embodiments, B and C are cysteine-containing peptides that are conjugated to each other via one or more Cys-Cys disulfide bonds B and C can be the same cysteine-containing peptides, or different cysteine-containing peptides. In some embodiments, each of B and C comprises the amino acid sequence SC (SEQ ID NO: 3).

In some embodiments, each of B and C contains a single cysteine. In some embodiments, each of B and C has any of 2, 3, or more cysteines. In some embodiments, B and C do not comprise cysteines that do not form disulfide bonds.

In some embodiments, DS1 and/or DS2 comprises a peptide substrate specific for an enzyme (e.g., protease) that is expressed at a disease site. In some embodiments, each of S1 and S2 comprises the amino acid sequence of SEQ ID NO: 3. In some embodiments, each of S1 and S2 comprises the amino acid sequence SCLSGRSDNH (SEQ ID NO:144). In some embodiments, each of S1 and S2 comprises the amino acid sequence SCLSGRSDNHGG (SEQ ID NO: 145). In some embodiments, each of S1 and S2 comprises the amino acid sequence SCLSGRSDNHGGGS (SEQ ID NO:146).

In some embodiments, the activatable antibody comprises a first polypeptide comprising the amino acid sequence of SEQ ID NO:25, and a second polypeptide comprising the amino acid sequence of SEQ ID NO: 26, wherein amino acid residue 2 (C) of SEQ ID NO: 25 is conjugated to amino acid residue 2 (C) of SEQ ID NO: 26 via Cys-Cys disulfide bond. In some embodiments, the activatable antibody comprises a first polypeptide comprising the amino acid sequence of SEQ ID NO: 27, and a second polypeptide comprising the amino acid sequence of SEQ ID NO. 28, wherein amino acid residue 2 (C) of SEQ ID NO: 27 is conjugated to amino acid residue 2 (C) of SEQ ID NO: 28 via Cys-Cys disulfide bond.

### 3. Unnatural Amino Acid Conjugation

As used herein, “unnatural amino acids” include any amino acids that do not occur or rarely occur in naturally produced proteins or polypeptides; that is, any amino acids that do not belong to the twenty conventional amino acids listed in the column “Original Residues” of Table 1 are considered “unnatural amino acids” for the purpose of this invention. In some embodiments, B and C comprise unnatural amino acids that are conjugated to each other via a covalent bond. In some embodiments, one of B and C comprises an unnatural amino acid, and the other comprises a natural amino acid, and wherein B and C are conjugated to each other via a covalent bond.

In some embodiments, B and C comprise selenocysteines which are conjugated via a diselenide bond. In some embodiments, one of B and C comprises a selenocysteine and the other comprises a cysteine, wherein the selenocysteine and the cysteine forms a Se—S bond. Selenocystine can be incorporated into proteins using any known methods in the art. For example, selenocysteine can be encoded by the UGA codon in an mRNA if a special stem-loop structure is present in the mRNA (Turanov A et al. 2013. Nucleic Acids Res. 41(14): 6952-6959).

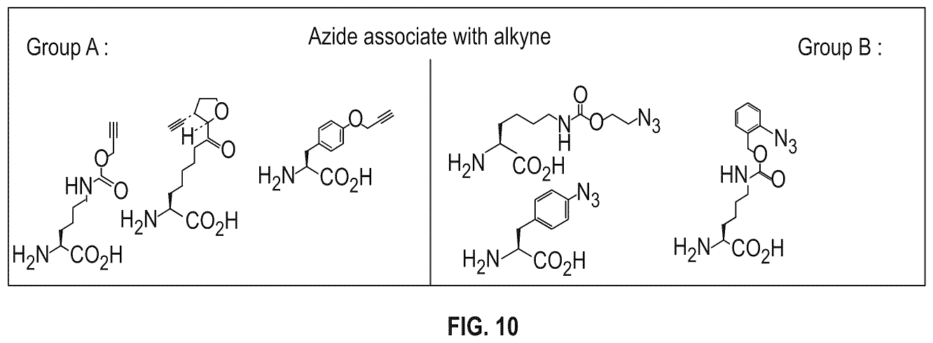

In some embodiments, B and C comprise an unnatural amino acid pair carrying side chains with orthogonal chemistries, such as those shown in FIG. 10. In some embodiments, one of B and C comprises an unnatural amino acid carrying an azide side chain, and the other comprises an unnatural amino acid carrying an alkyne group, wherein B and C are conjugated to each other via click-chemistry. In some embodiments, one of B and C comprises any one of the unnatural amino acids in group A of FIG. 10, and the other comprises any one of the unnatural amino acids in group B of FIG. 10. For example, B comprises p-Propargyloxyphenylalanine and C comprises p-Azidophcnylalanine. Other orthogonal unnatural amino acid pairs known in the art can also be used in the present application. See, for example, Liu C C and Schultz P G. 2010 Annual Review of Biochemistry. 79-413-444 Unnatural amino acids can be genetically incorporated into proteins expressed in mammalian or E. coli expression systems using known approaches in the art. See, for example, Liu C C and Schultz P G. 2010. Annual Review of Biochemistry. 79: 413-444.

### B. Non-Covalent Association

In some exemplary activatable antibodies, the association between S1 and S2 is non-covalent. In some embodiments, wherein S1 comprises a first association moiety (B), and S2 comprises a second association moiety (C), and wherein B and C interact with each other non-covalently. In some embodiments, B is further conjugated to C covalently, such as with any one of the manners described in Section A.

In some embodiments, B and C are proteins, domains or fragments, or complementary binding peptides. For example, B and C can each be a half fragment of a split-protein, a monomeric subunit of a protein dimer, one of a pair of complementary binding peptides, or one of VH and VL of an Fv fragment. B and C can be genetically encoded and engineered into the activatable antibodies using any known methods for producing antibody fusion proteins known in the art. B and C can form a functional protein or protein domain thereby providing the steric shield in the activatable antibody. See, e.g., FIGS. 21-2D. In some embodiments, one of B and C is a protein (e.g., lectin), and the other is a naturally occurring chemical moiety (e.g., glycan) that the protein binds to. See. e.g., FIG. 2E. The naturally occurring chemical moiety can be post-translationally added onto the polypeptide either during antibody expression or in vitro using an enzyme, thereby allowing formation of the non-covalent association between B and C.

The distance spanned by the folded S1 and S2 is sufficiently short to prevent antigen binding by the ABD in the activatable antibody. This distance can be controlled by the number of residues of disease-sensing releasable moieties DS1 and DS2 that act as releasable linkers between the N-termini of VH and VL of the antibody and the C-termini of the folded B and C moieties. This distance can be adjusted according to antigen binding efficiency data. e.g., assessed using surface plasmon resonance methods as described in Example 9.

Non-limiting examples of B and C that allow non-covalent association between S1 and S2 are described in sections 1-5 below %.

### 1. Split-Protein

In some embodiments, B and C are two complementary halves of a split-protein. Single-domain proteins can be split into two polypeptides that associate to form a whole protein. These two half fragments can be fused to the N-termini of VH and V L of the ABO respectively. The two half fragments associate non-covalently to form a steric shield comprising a whole protein, thereby joining the N-termini of the V-H and VL of the ABD. See, e.g., FIG. 2B.

In some embodiments, the split-protein is selected from the group consisting of GFP, Ubiquitin, CnaB2, interleukins, and chemokines. In some embodiments, S1 comprises the amino acid sequence of SEQ ID NO: 4 and S2 comprises the amino acid sequence of SEQ ID NO: 5, or S2 comprises the amino acid sequence of SEQ ID NO. 4 and S1 comprises the amino acid sequence of SEQ ID NO: 5. In some embodiments. S1 comprises the amino acid sequence of SEQ ID NO: 6 and S2 comprises the amino acid sequence of SEQ ID NO: 7, or S2 comprises the amino acid sequence of SEQ ID NO: 6 and S1 comprises the amino acid sequence of SEQ ID NO: 7.

In some embodiments, the activatable antibody comprises a first polypeptide comprising the amino acid sequence of SEQ ID NO. 39, and a second polypeptide comprising the amino acid sequence of SEQ ID NO: 40.

Any split-proteins known in the art can be used in the present application, including, but not limited to those listed in Table 3 below. Split-proteins have previously used to study protein-protein interactions. For example, GFP. Ubiquitin. CnaB2 domain from FbaB of Streptococcus pyaogenes, interleukins, and chemokines (such as CC; CXC; C; CX3C families) have been designed as split-proteins. See, for example, Johnsson, N. & Varshavsky, A. 1994. Proc. Natl. Acad. Sci USA 91, 10340-10344; Zakeri B et al. 2012. PNA. 109.690-697: Li L et al. 2014. J Mol Biol. 426.30)-317.

TABLE 3
Exemplary half fragments of split-proteins.

| SEQ | | |
|---|---|---|
| ID | | |
| NO | Source | Sequence |
| 4 | CnaB2 | DSATHIKFSKRDEDGKELAGATMELRDSSGKTISTWISDGQVKDF |
| | split- | YLYPGKYTFVE TAAPDGYEVATAITFTVNEQGQVTVNG |
| | protein | (Spy Catcher) |
| 5 | | AHIVMVAAYKPTK (Spytag) |
| 6 | Ubiquitin | MQIFVKTLTGKTITLEVEPSDTIENVKAKIQDKE (Cub) |
| 7 | split- | GIPPDQQRLIFAGKQLEDGRTLSDYNIQKESTLHLVLRLRGG (Nub) |
| | protein | |

### 2. Dimeric Protein

In some embodiments, B and C are two monomeric subunits of a protein dimer. In some embodiments. B and C have the same sequence. In some embodiments, B and C are associated to form a homodimeric protein in some embodiments, B and C have different sequences. In some embodiments, B and C are associated to form a heterodimeric protein. In some embodiments, the dimeric protein is a domain-swapped dimer. These two monomeric subunits can be fused to the N-termini of VH and V L of the ABD respectively. During protein expression, the monomeric subunits associate with each other non-covalently to form a homo- or hetero-dimeric protein, thereby joining the N-termini of the VH and VL of the ABD. See, e.g., FIG. 2C.

In some embodiments, the monomeric subunits in an activatable antibody are identical. In some embodiments, the monomeric subunits are different. In some embodiments, each of the monomeric subunits comprises an amino acid sequence selected from the group consisting of SEQ ID NOs: 8-10. In some embodiments, the first monomeric subunit comprises the amino acid sequence of SEQ ID NO: 11 and the second monomeric subunit comprises the amino acid sequence SEQ ID NO: 12, or the second monomeric subunit comprises the amino acid sequence of SEQ ID NO. 11 and the first monomeric subunit comprises the amino acid sequence SEQ ID NO: 12.

In some embodiments, the activatable antibody comprises a first polypeptide comprising the amino acid sequence of SEQ ID NO. 37, and a second polypeptide comprising the amino acid sequence of SEQ ID NO: 38. In some embodiments, the activatable antibody comprises a first polypeptide comprising the amino acid sequence of SEQ ID NO: 41, and a second polypeptide comprising the amino acid sequence of SEQ ID NO:42.

Any of the known dimeric proteins in the art can be used in the present application, including, but not limited to those listed in Table 4 below. In some embodiments, the dimeric protein is selected from the group consisting of HIV protease, relaxin-2, Myo10, STIL, Troponin C, defensin-5, IL-10. DHBN domain of human BLM helicase, Insulin, human copper chaperone, KIX domain of human RECQL5, galectin-1. TNF-alpha, human neutrophil peptide 1, CXCL5, CXCL4, ILT1-immunoglobulin-like transcript, and IL-12 See, for example, Zdanov et al. 1995. Structure. 3: 591-601.

TABLE 4
Exemplary monomeric subunits of protein dimers.

| SEQ | | |
|---|---|---|
| ID | | |
| NO | Source | Sequence |
| 8 | IL-10 | SPGQGTQSENSCTHFPGNLPNMLRDLRDAFSRVKTFFQMKDQLDNLL |
| | | LKESLLEDFKGYLGCQALSEMIQFYLEEVMPQAENQDPDIKAHVNSL |
| | | GENLKTLRLRLRRCHRFLPCENKSKAVEQVKNAFNKLQEKGIYKAMS |
| | | EFDIFINYIEAYMTMKIRN |
| 9 | CXCL4 | EAEEDGDLQCLCVKTTSQVRPRHITSLEVIKAGPHCPTAQLIATLKNG |
| | | RKICLDLQAPLYKKIIKKLLES |
| 10 | CXCLS | AGPAAAVLRELRCVCLQTTQGVHPKMISNLQVFAIGPQCSKVEVVAS |
| | | LKNGKEICLDPEAPFLKKVIQKILDGGNKEN |
| 11 | IL-12 | MCPARSLLLVATLVLLDHLSLARNLPVATPDPGMFPCLHHSQNLLRA |
| | | VSNMLQKARQTLEFYPCTSEEIDHEDITKDKTSTVEACLPLELTKNES |
| | | CLNSRETSFITNGSCLASRKTSFMMALCLSSIYEDLKMYQVEFKTMNA |
| | | KLLMDPKRQIFLDQNMLAVIDELMQALNFNSETVPQKSSLEEPDFYK |
| | | TKIKLCILLHAFRIRAVTIDRVMSYLNAS (Subunit A) |
| 12 | | MCHQQLVISWFSLVFLASPLVAIWELKKDVYVVELDWYPDAPGEMV |
| | | VLTCDTPEEDGITWTLDQSSEVLGSGKTLTIQVKEFGDAGQYTCHKG |
| | | GEVLSHSLLLLHKKEDGIWSTDILKDQKEPKNKTFLRCEAKNYSGRFT |
| | | CWWLTTISTDLTFSVKSSRGSSDPQGVTCGAATLSAERVRGDNKEYE |
| | | YSVECQEDSACPAAEESLPIEVMVDAVHKLKYENYTSSFFIRDIIKPDP |
| | | PKNLQLKPLKNSRQVEVSWEYPDTWSTPHSYFSLTFCVQVQGKSKRE |
| | | KKDRVFTDKTSATVICRKNASISVRAQDRYYSSSWSEWASVPCS |
| | | (Subunit B) |

### 3. Complementary Biding Peptides

In some embodiments, B and C are complementary binding peptides. In some embodiments. B and C are associated to form a parallel coiled-coil homo dimers. In some embodiments. B and C are associated to form an anti-parallel coiled-coil heterodimers. In some embodiments. B and C are associated to form a leucine zipper. In some embodiments, B and C have the same sequence. In some embodiments, B and C have different sequences. The two complementary binding peptides can be fused to the N-termini of VH and VL of the ABD respectively. During protein expression, the complementary binding peptides associate with each other non-covalently to form a homo- or hetero-dimer complex, thereby joining the N-termini of the VH and VL of the ABD. See, e.g., FIG. 2D.

In some embodiments, the first complementary binding peptide and the second complementary binding peptide are identical. In some embodiments, the first complementary binding peptide and the second complementary binding peptide are different. In some embodiments, each of the first complementary binding peptide and the second complementary binding peptide comprises an amino acid sequence selected from the group consisting of SEQ ID NOs: 13-15. In some embodiments, the first complementary binding peptide comprises the amino acid sequence of SEQ ID NO: 16 and the second complementary binding peptide comprises the amino acid sequence SEQ ID NO: 17, or the second complementary binding peptide comprises the amino acid sequence of SEQ ID NO. 16 and the first complementary binding peptide comprises the amino acid sequence SEQ ID NO: 17. In some embodiments, the activatable antibody comprises a first polypeptide comprising the amino acid sequence of SEQ ID NO: 43, and a second polypeptide comprising the amino acid sequence of SEQ ID NO: 44.

Any of the known complementary binding peptides in the art can be used in the present application, including, but not limited to those listed in Table 5 below.

TABLE S
Exemplary complementary binding peptides that form coiled coils.

| SEQ | | |
|---|---|---|
| ID | | |
| NO | Source | Sequence |
| 13 | Myo10 | ENKQVEEILRLEKEIEDLQRMKEQQELSLT |
| 14 | JIP3 | GAMDPEFTKNALNVVKNDLIAKVDQLSGEQEVLRGELEAAKQAK |
| | | VKLENRIKELEEELKRV |
| 15 | KIF21A | GSMTISNMEADMNRLLKQREELTKRREKLSKRREKIVKENGEGD |
| | | KNVANINEEMESLTANIDYINDSISDCQANIMQMEE |
| 63 | | KLEALEGKLEALEKLEALEGKLEALEGKLEALEG |
| 16 | MST1- | GSDYEFLKSWTVEDLQKRLLALDPMMEQEIEEIROKYQSKRQPIL |
| | RASSF5 | DAIEAK (Part A) |
| 17 | SARAH | GSEVEWDAFSIPELQNFLTILEKEEQDKIQQVQKKYDKFRQKLEEA |
| | | LRESQGKPG (Part B) |
| 64 | E.coli ProP | DNIEQKIDDIDHEIADLQAKRTRKVQQHPR |
| 65 | 468-497 | RPHQQVLRTRKAQLDAIEHDIDDIKQEIND |
| 66 | | RRRRRRRRRRR |
| 67 | | EEEEEEEEEEE |

### 4. Fv Fragments

In some embodiments, one of B and C comprises a second VH, and the other comprises a second VL, and wherein B and C are associated to form an Fv. In some embodiments, B comprises a second VH and C comprises a second VL. In some embodiments, B comprises a second VL and C comprises a second VH. The second VH and VL can be fused to the N-termini of VH and VL of the ABD, either in the form of VH2-VL1 and VL2-VH1, or VH2-VH1 and VL2-VL1. In some embodiments, each of B and C comprises a cysteine, and wherein B and C are conjugated to each other via a disulfide bond Fv fusions to antibodies, including those with disulfide enhancement for the Fv domains are known in the art to provide multispecific antibodies, in which the Fv domain does not block the antigen binding sites of the antibodies. See. e.g., DVD-Igs described in WO2007024715. While the fusion techniques known for DVD-Igs are applicable to the present application, the Fv in the activatable antibodies described herein sterically blocks antigen binding of the ABD. This can be achieved by shortening or omitting any peptide linkers between the second VH or VL and the N-terminus of the VH or VL of the ABD. In some embodiments, one or more (such as 1, 2, 3, 4, or 5) native N-terminal residues of the FR1 region of VH and/or VL of the ABD are deleted to enhance the steric hindrance of the Fv on the antigen binding site.

In some embodiments, the second VH comprises the amino acid sequence of SEQ ID NO: 35, and the second VL comprises the amino acid sequence of SEQ ID NO: 36. In some embodiments, the activatable antibody comprises a first polypeptide comprising the amino acid sequence of SEQ ID NO: 45, and a second polypeptide comprising the amino acid sequence of SEQ ID NO: 46. In some embodiments, disruption (e.g., cleavage) of DS1 and/or DS2 unblocks the binding of the ABD to its target. In some embodiments, DS1 and/or DS2 comprises a peptide substrates specific for an enzyme (e.g., protease) that is expressed at a diseased site. In some embodiments. DS1 and/or DS2 comprises a pH-sensitive protein or peptide. In some embodiments, the Fv is a therapeutic agent.

The VH and VL of any suitable Fv fragments can be used in the present application. In some embodiments, the second VH and second VL are derived from the L19 antibody as shown in Table 6. In some embodiments, the Fv is a therapeutic agent. In some embodiments, the Fv targets any one of the molecules listed in Table 8. In some embodiments, the Fv is derived from any one of the known therapeutic antibodies in the art, such as those listed in Table 9.

TABLE 6
Exemplary VH and VL sequences of therapeutic Fv.

| SEQ | | |
|---|---|---|
| ID | | |
| NO | Target | Sequence |
| 35 | ED-B | EVQLLESGGGLVQPGGSLRLSCAASGFTFSSFSMSWVRQAPGKGL |
| | | EWVSSISGSSGTTYYADSVKGRFTISRDNSKNTLYLQMNSLRAED |
| | | TAVYYCAKPFPYFDYWGQGTLVTV (VH) |
| 36 | | EIVLTQSPGTLSLSPGERATLSCRASQSVSSSYLAWYQQKPGQAPR |
| | | LLIYYASSRATGIPDRfSGSGSGTDFTLTISRLEPEDFAVYYCQQTG |
| | | RIPPTFGQGTKVEI (VL) |

### 5. Glycan-Binding Protein-Glycan Complex

In some embodiments, one of B and C is a glycan linked to a glycosylation peptide sequence, and the other is a glycan-binding protein. In some embodiments, the glycan-binding protein is a lectin, ficolin, or ERGIC-53. Both the glycosylation peptide sequence and the lectin can be genetically fused to the N-termini of the VH and VL of the ABD. During expression of the antibody in mammalian cells or other cells (e.g., yeast) expressing engineered glycosylases, the glycosylation peptide sequence is glycosylated, allowing formation of a glycan-binding protein-glycan complex to sterically block the antigen binding site of the ABD. In some embodiments, the conformation of the glycan-binding protein is sensitive to change in pH, such as acidic pH. Thus, the glycan-binding protein can serve as DS1 or DS2. For example, at acidic pH environment in a tumor tissue, the glycan-binding protein has a conformational change, thereby releasing the glycan, and disrupting the steric shield. In some embodiments, DS1 and/or DS2 comprise disease-specific protease substrates, which allow cleavage and release of the glycan-binding protein-glycan complex from the antigen-binding site.

In some embodiments, the glycan-binding protein is a lectin, ficolin, or ERGIC-53. In some embodiments, the glycan-binding protein is M-ficolin, such as an engineered M-ficolin monomer, and the glycan is GlcNAc. In some embodiments, the glycan-binding protein comprises the amino acid sequence of SEQ ID NO:20. In some embodiments, the glycosylation peptide sequence comprises the amino acid sequence of SEQ ID NO: 18. In some embodiments, the glycan-binding protein is M-ficolin. In some embodiments, the activatable antibody comprises a first polypeptide comprising the amino acid sequence of SEQ ID NO: 47, and a second polypeptide comprising the amino acid sequence of SEQ ID NO:48. In some embodiments, the activatable antibody comprises a first polypeptide comprising the amino acid sequence of SEQ ID NO: 49, and a second polypeptide comprising the amino acid sequence of SEQ ID NO: 50.

Any suitable glycan-binding protein-glycan pair known in the art can be used in the present application. In some embodiments, the glycan-binding protein is a lectin, ficolin, or ERGIC-53. In some embodiments, the glycosylation peptide sequence comprises one or more (such as any of 1, 2, 3, or more) glycosylation sites. In some embodiments, one or more (such as any of 1, 2, 3, or more) glycans are added to the glycosylation peptide sequence. In some embodiments, the glycosylation peptide sequence comprises an N-glycosylation site. In some embodiments, the glycosylation peptide sequence comprises an O-glycosylation site. Exemplary glycosylation peptide sequences are listed in Table 7. In some embodiments, the glycosylation peptide sequence has an amino acid sequence selected from SEQ ID NOs: 18, and 68-73.

In some embodiments, the glycans are added during expression of the fusion antibody in mammalian cells or other cells (e.g., yeast) expressing engineered glycosylases. In some embodiments, the glycans are added to the expressed fusion antibody in vitro by treating the fusion antibody with a glycosylase. In some embodiments, the antibody carrying the N-terminal glycosylation peptide sequence has its native glycosylation site(s) mutated to abolish the native glycosylation sites. For example, an amino acid substitution at residue Asn297 can be introduced to the antibody.

TABLE 7
Exemplary glycosylation tag and glycan-binding protein sequences.

| SEQ ID | | |
|---|---|---|
| NO | Source | Sequence |
| 68 | N- | NXS/T/C, where X is any amino acid with exception of P |
| | glycosylation | |
| | tag | |
| 18 | SLC6A1 | SNYSMVNTTNMTSA |
| 69 | ABCB11 | VNNTIVWTNSSLNQNMTNGT |
| 70 | FactorX | FNQTQPERGDNNLTR |
| 71 | CFTR | GNSTHSRNNSY |
| 72 | IgG | EEQYNSTYR |
| 73 | IgG | EEQYNSTFR |
| 20 | Monomeric | QSCATGPRNCKDLLDRGYFRSGWHTIYLPDCRPLTVLCDMDTDGGG |
| | M-Ficolin | WTVFQRRMDGSVDFYRDWAAYKQGFGSQLGEFWLGNDNIHALTAQ |
| | with site- | GSSELRTDLVDFEGNHQFAKYKSFKVADEAEKYKLVLGAFVGGSAGN |
| | specific | SLTGHNNNFFSTKDQDNDVSSSNCAEKFQGAWWYADCHASNLNGLY |
| | mutation | LMGPHESYANGINWSAAKGYKYSYKVSEMKVRPA |
| | (bold letter) | |

### C. Disease-Sensing Releasable Moieties

S1 and/or S2 comprises a disease-sensing releasable moiety. In some embodiments, S1 comprises DS1 but S2 does not comprise DS2. In some embodiments, S2 comprises DS2 but S1 does not comprise DS1. In some embodiments, S1 comprises DS1 and S2 comprises DS2. In some embodiments, DS1 is identical to DS2. In some embodiments, DS1 is different from DS2.

As used herein, “disease-sensing releasable moiety” refers to polypeptides or chemical groups that are sensitive to a specific chemical or physical element present in a diseased tissue. For example, many diseased tissues are known to differ from normal tissue by pH, oxygen level, and expression of enzymes such as disease-specific proteases. The disease-sensing releasable moiety is disrupted, such as cleaved or undergone conformational changes, by the chemical or physical element in the diseased tissue, thereby releasing the steric shield and unblocking the antigen binding site. Each of DS1 and DS2 can sense one or more chemical or physical elements specific to the diseased tissue. For example, each of DS1 and DS2 may comprise a single disease-specific protease substrate, or two or more substrates that respond to different disease-specific proteases. In some embodiments, DS1 and/or DS2 can respond to acidic pH and be cleaved by a disease-specific protease. In some embodiments, DS1 and DS2 sense the same specific chemical or physical element in a diseased tissue. In some embodiments, DS1 and DS2 sense different chemical or physical elements in a diseased tissue. For example, one of DS1 and DS2 is a pH sensor, and the other is a protease-cleavable sensor. A skilled person in the art would readily appreciate that the different disease-sensing releasable moieties described herein can be mixed and matched to provide activatable antibodies with different activation mechanisms and dynamics.

In some embodiments, DS1 and/or DS2 comprises at least two (such as any of 2, 3, 4, 5, or more) peptide substrates each specific for an enzyme that is expressed at a diseased site. In some embodiments. DS1 and/or DS2 comprises a peptide linker, such as a flexible peptide linker that connects between the various peptide substrates.

In some embodiments, DS1 and/or DS2 is a tumor-sensing releasable moiety. Tumors differ from normal tissues in several physiological and micro-environmental characteristics. For example, hypoxia and extracellular acidity are hallmark of malignant tumors. Tumor hypoxia indicates that within tumors significant areas have lower oxygen tension that found in normal tissues. pH of extracellular compartment in tumors is lower than commonly observed in normal tissues.

### 1. Disease-Specific Protease Sensors

In some embodiments, DS1 and/or DS2 is a protease substrate cleavable by a disease-specific protease, such as a tumor-specific protease. In some embodiments, the tumor-specific protease express at elevated levels in diseased tissues, such as tumor tissues, compared to normal tissues. For example, studies have shown that tumor tissues exhibit increased activity of specific proteases and decreased activity of the opposing endogenous inhibitors (Sevenich L and Joyce J A. Genes & Dev. 2014. 28: 2331-2347, 2014). In some embodiments, tumor-specific protease is selected from the group consisting of matriptase (MTSP1), urinary-type plasminogen activator (uPA), legumain. PSA (also called KLK3, kallikrein-related peptidase-3), matrix metalloproteinase-2 (MMP-2), matrix metalloproteinase-9 (MMP9), human neutrophil elastase (HNE), proteinase 3 (Pr3), cathepsin B and cathepsin K.

In some embodiments, DS1 and/or DS2 comprises a peptide substrate of matriptase. Any known matriptase cleavage sequence can be used, for example, RQARVV (SEQ ID NO. 147); X1SGRSA (SEQ ID NO: 148), wherein X1 is G or K; LSGRSX1NH (SEQ ID NO: 154) wherein X1 is D, V or A; X1X2X3X4X5X6X7X8 (SEQ ID NO: 149), wherein X1 is R, X2 and X3 are any amino acid, X4 is R, X5 is K, X6 is V, X7 is any amino acid, X8 is G; X9X10X11X12X13 (SEQ ID NO: 150), wherein X9 is K or R, X10 is R, K. H or Q, X11 is G, A or S, X12 is R or K, X13 is A; X14X15X16X17X18X19X20X21 (SEQ ID NO: 151), wherein X14 is L, I, A or K, X15 is S, G, or Q, X16 is K, L, A, G, or S, X17 is R, X18 is S, K, G, A or V, X19 is A, V or L, X20 is N, X21 is H, G, V, or L; X22X23X24X25X26 (SEQ ID NO: 152), wherein X22 is R or K, X23 is non-basic amino acid (G, A, S, T, C, V, L, I, M, P, F, Y, W, D, E, N, or Q), X24 is S, X25 is R, X26 is A; or X27X28X29X30X31 (SEQ ID NO: 153), wherein X27 is non-basic amino acid (G, A, S, T, C, V, L, I, M, P, F, Y, W, D, E, N, Q), X28 is R or K, X29 is S, X30 is R, X31 is A.

In some embodiments, DS1 and/or DS2 comprises a peptide substrate of legumain. Any known legumain cleavage sequence can be used, for example, LSGRSX1NH (SEQ ID NO: 154), wherein X1 is D, V or A; X1X2X3X4X5X6X7X8 (SEQ ID NO: 155), wherein X1, X2, X3 are any amino acid, X4 is N, X5, X6, X7, X8 are any amino acid: AANL (SEQ ID NO: 156): ATNL (SEQ ID NO: 157); or X9X10X11X12X13X14X15X16 (SEQ ID NO: 158), wherein X9 is L, I, A or K, X10 is S, G, or Q, X11 is K, L, A, G, or S, X12 is R, X13 is S, K, G, A or V, X14 is A, V or L, X15 is N, X16 is H, G, V, or L.

In some embodiments, DS1 and/or DS2 comprises a peptide substrate of uPA (Urinary-type plasminogen activator). Any known uPA cleavage sequence can be used, for example, X1SGRSA (SEQ ID NO: 159), wherein X1 is G or K; LSGRSX1NH (SEQ ID NO: 154) wherein X1 is D. V or A; X1X2X3X4X5X6X7X8 (SEQ ID NO: 160), wherein X1 is any amino acid, X2 is S, X3 is G or S, X4 is R or K, X5 is any amino acid, X6 is R or V, X7 is any amino acid, X8 is any amino acid; SGRSA (SEQ ID NO: 161); X9X10X11X12X13X14X15X16 (SEQ ID NO. 162) wherein X9 is L, I, A or K, X10 is S, G, or Q, X11 is K, L, A, G, or S, X12 is R, X13 is S, K, G, A or V, X14 is A, V or L, X15 is N, X16 is H, G, V, or L; or RKSSHIRMRDWL (SEQ ID NO: 163).

In some embodiments, DS1 and/or DS2 comprises a peptide substrate of Pro-urokinase Any known Pro-uPA cleavage sequence can be used, for example, PRFX1HGG (SEQ ID NO: 164), wherein X1 is K or R.

In some embodiments, DS1 and/or DS2 comprises a peptide substrate of matrix metalloprotease. Any known matrix metalloprotease cleavage sequence can be used, for example, SRPLALR (SEQ ID NO: 165); SRPANLR (SEQ ID NO: 166); or X1X2X3X4X5X6X7 (SEQ ID NO: 167), wherein X1 is S, V or R, X2 is any amino acid, X3 is P or A, X4 is L, M, A, R or Y, X5 is A, S, N, G, H or M, X6 is L, X7 is R, L, Q or M.

In some embodiments, DS1 and/or DS2 comprises a peptide substrate of MMP1 (Human fibroblast collagenase). Any known MMP1 cleavage sequence can be used, for example, DVAQFVLT (SEQ ID NO: 168): or X1X2X3X4X5X6X7X8 (SEQ ID NO. 169), wherein X1 is any amino acid, X2 is P or A, X3 and X4 are any amino acids, X5 is L or 1, X6, X7, X8 are any amino acids.

In some embodiments, DS1 and/or DS2 comprises a peptide substrate of MMP2. Any known MMP2 cleavage sequence can be used, for example, HRPRGX1TN (SEQ ID NO: 170), wherein X1 is V or W; X1X2X3X4X5X6X7X8 (SEQ ID NO: 171), wherein X1 is any amino acid, X2 is P, X3 and X4 are any amino acids, X5 is L or 1, X6, X7 and X8 are any amino acids: or PLGLAG (SEQ ID NO: 172).

In some embodiments, DS1 and/or DS2 comprises a peptide substrate of MMP9. Any known MMP9 cleavage sequence can be used, for example, HRPRGX1TN (SEQ ID NO: 173), wherein X1 is V or W; X1X2X3X4X5X6X7X8 (SEQ ID NO: 174), wherein X1 is G, X2 is P or A, X3 is any amino acids, X4 is G or A, X5 is L, X6 is any amino acid, X7 is G, X8 is any amino acid; GPLGIAGQ (SEQ ID NO: 175); PVGLIG (SEQ ID NO: 176): or HPVGLLAR (SEQ ID NO: 177).

In some embodiments, DS1 and/or DS2 comprises a peptide substrate of Gelatinase. Any known Gelatinase cleavage sequence can be used, for example, PLGLWA (SEQ ID NO: 178): or X1X2X3X4X5X6X7 (SEQ ID NO: 179), wherein X1 is H, K, R or V, X2 is R, M, T, V, Y, or A, X3 is P or V, X4 is R, S or A, X5 is G, A, S, N or W, X6 is V, W, L or Y, X7 is T, R, Y, I.

In some embodiments, DS1 and/or DS2 comprises a peptide substrate of PSA. Any known PSA cleavage sequence can be used, for example, SSKLQ (SEQ ID NO: 180); SRKSQQY (SEQ ID NO: 181); GQKGQHY (SEQ ID NO: 182), KGISSQY (SEQ ID NO. 183); X1X2X3X4X5X6X7X8 (SEQ ID NO: 184), wherein X1 is S or I, X2 is S or Q, X3 is any amino acid, X4 is Y, Q, or E, X5 is S, X6 is S, X7 and X8 are any amino acid: or SSX1YSX2 (SEQ ID NO: 185), wherein X1 is Y or F and X2 is G or S.

In some embodiments, DS1 and/or DS2 comprises a peptide substrate of human neutrophil elastase Any known human neutrophil elastase cleavage sequence can be used, for example, X1X2X3X4X5X6X7X8 (SEQ ID NO: 186), wherein X1, X2 and X3 are any amino acid, X4 are V, I, A or T, X5, X6, X7, X8 are any amino acid; X9EX10VVY (SEQ ID NO: 187), wherein X9 is R or M, X10 is A or H; VADCAQ (SEQ ID NO: 188): APEEIMDRQ (SEQ ID NO: 189); IVSARMAPEEIIMDRQ (SEQ ID NO: 190); or GIATFCMLMPEQ (SEQ ID NO 191).

In some embodiments. DS1 and/or DS2 comprises a peptide substrate of proteinase 3. Any known proteinase 3 cleavage sequence can be used, for example, X1X2X3X4X5X6X7X8 (SEQ ID NO: 192), wherein X1 is any amino acid X2 is Y, X3 is Y, X4 is V, T or A, X5, X6. X7, X8 are any amino acids: VADCAQ (SEQ ID NO: 193). APEEIMDRQ (SEQ ID NO: 194); IVSARMAPEEIIMDRQ (SEQ ID NO: 195): or GIATFCMLMPEQ (SEQ ID NO: 196).

In some embodiments, DS1 and/or DS2 comprises a peptide substrate of Factor Xa. Any known Factor Xa cleavage sequence can be used, for example, IEGR (SEQ ID NO: 197); IDGR (SEQ ID NO: 198); GGSIDGR (SEQ ID NO: 199): or X1X2X3X4X5X6X7X8 (SEQ ID NO: 200), wherein X1 is A or 1, X2 is any amino acid, X3 is G, P or F, X4 is R, X5 is T, S or 1, X6 is V or F, X7 is any amino acid, X8 is G.

In some embodiments, DS1 and/or DS2 comprises a peptide substrate of cathepsin B. Any known cathepsin B cleavage sequence can be used, for example, X1X2X3GX4GX5 (SEQ ID NO: 201), wherein X1, X2, X3, X4 and X5 are any amino acid.

In some embodiments, DS1 and/or DS2 comprises a peptide substrate of cathepsin K. Any known cathepsin K cleavage sequence can be used, for example, X1X2X3X4X5X6X7X8 (SEQ ID NO: 202), wherein X1, X2, X5, X6, X7 and X8 are any amino acids, and X3 is L, P or V, and X4 is E or A.

In some embodiments, DS1 and/or DS2 comprise a single peptide substrate cleavable by a disease-specific protease, such as any one of SEQ ID NOs: 147-202. In some embodiments, DS1 and/or DS2 comprises two or more copies (such as any one of 2, 3, 4, 5, or more) of a peptide substrate cleavable by a disease-specific protease. In some embodiments, DS1 and/or DS2 comprises a peptide substrate that can be cleaved by more than one (e.g., any of 2, 3, 4, or more) disease-specific proteases. In some embodiments, DS1 and/or DS2 comprises two or more (e.g., any of 2, 3, 4, or more) peptide substrates cleavable by one or more (e.g., any of 2, 3, 4, or more) disease-specific proteases. Any of the protease peptide substrate sequences disclosed herein (e.g., SEQ ID NOs: 19 and 147-202) can be mixed and matched to provide a disease-sensing releasable moiety with optimal mechanism and dynamics for release of the steric shield in a diseased site. The different protease substrate sequences or copies thereof can be fused to each other via peptide linkers to provide suitable disease-sensing releasable moieties.

In some embodiments, DS1 and/or DS2 comprises the amino acid sequence of LSGRSDNH (SEQ ID NO: 19). Tbc protease substrate LSGRSDNH (SEQ ID NO: 19) is sensitive to numerous proteases that are activated and/or upregulated in a variety of human carcinomas, including matriptase, urinary-type plasminogen activator (uPA), and legumain. In some embodiments. DS1 and/or DS2 comprises the amino acid sequence of PLGLAG (SEQ ID NO: 172). The protease substrate PLGLAG (SEQ ID NO: 172) is sensitive to matrix metalloproteases that are activated and/or upregulated in a variety of human carcinomas. In some embodiments, DS1 and/or DS2 comprises the amino acid sequence of KSRTTNG (SEQ ID NO: 240) or KGSRTTNG (SEQ ID NO: 241). The protease substrates KSRTTNG (SEQ ID NO: 240) and KGSRTTNG (SEQ ID NO 241) are sensitive to matriptase and legumain.

In some embodiments, DS1 and/or DS2 comprises a peptide with unnatural amino acids that can be cleaved by a disease-specific protease. In some embodiments, DS1 and/or DS2 comprises valine-citrulline (SEQ ID NO: 2(4), which can be cleaved by cathepsin B.

### 2. pH Sensors.

In some embodiments, DS1 and/or DS2 is a pH sensing moiety. In some embodiments, DS1 and/or DS2 is disrupted by low pH. Studies have shown that tumor cell mediated oncogenic metabolism generates a large amount of lactic acid and protons, leading to the reduction in the extracellular pH values to as low as 6 in tumor tissue (Icard et al., Biochim. Biophys. Acta. 1826:423433, 2012). Thus, in the healthy tissues where pH concentration is higher, the N-termini of activatable antibodies have a low-pH sensor as DS1 and/or DS2 remain linked, while in the diseased tissue, under acidic pH conditions. DS1 and/or DS2 is cleaved, thereby releasing the steric shield and unblocking the antigen biding sites.

In some embodiments, DS1 and/or DS2 are proteins that undergo conformational changes as pH shifts from neutral towards acidic. For example, histidines within proteins can act as pH sensor and thus mediate pH-controlled structural changes. In some embodiments, the association moieties B and/or C are pH-sensitive proteins, and thus also serve as disease-sensing releasable moieties.

In some embodiments, one of each of DS1 and DS2 are pH-sensitive peptides. Any known pH-sensitive peptides can be used in the present application, for example, the GALA comprising the amino acid sequence of WEAALAEALAEALAEHLAEALAEALEALAA (SEQ ID NO: 203), coiled-coil domain of macrophage scavenger receptor (Doi et al 1994. J. Biol Chem. 269: 25598-25604), or coiled-coil domains of Emp46p and Emp47p (Ishi K et al. 2015. PLoS ONE 10(10): e014028).

### D. Peptide Linkers

In some embodiments, S1 and/or S2 comprises one or more peptide linkers that connect between the various components in the activatable antibody. In some embodiments, the N-terminus of the VH is fused to the C-terminus of S1 via a peptide linker. In some embodiments, the N-terminus of the VL is fused to the C-terminus of S2 via a peptide tinker. In some embodiments. DS1 is fused to B via a peptide linker. In some embodiments, DS2 is fused to C via a peptide linker. In some embodiments, different protease substrate sequences within DS1 are linked to each other via a peptide linker.

The peptide linker can have any suitable sequences and of any suitable length. In some embodiments, the peptide linker is about any one of 1, 2, 3, 4, 5, 6, 7, 8, 9, 10, 12, 15, 20 amino acids long. In some embodiments, the peptide linker is about 1-12 amino acids long. In some embodiments, the peptide linker is a flexible peptide linker containing flexible amino acid residues, such as glycine and serine. In some embodiments, the peptide linker comprises a motif. e.g., multiple or repeating motifs, of GS, GGS (SEQ ID NO: 205), GGGGS (SEQ ID NO: 206), GGSG (SEQ ID NO: 207). SGGG (SEQ ID NO: 208), or GSGGGS (SEQ ID NO: 226). In some embodiments, the peptide linker comprises 1 to 12 amino acids including motifs of GS. e.g., GS, GSGS (SEQ ID NO: 209), GSGSGS (SEQ ID NO: 210), GSGSGSGS (SEQ ID NO: 211), GSGSGSGSGS(SEQ ID NO: 212), or GSGSGSGSGSGS (SEQ ID NO: 213). In some embodiments, the peptide linker comprises 3 to 12 amino acids including motifs of GGS (SEQ ID NO 205), e.g., GGS (SEQ ID NO: 205), GGSGGS (SEQ ID NO: 214). GGSGGSGGS (SEQ ID NO: 215), or GGSGGSGGSGGS (SEQ ID NO. 216). In some embodiments, the peptide linker comprises 4 to 12 amino acids including motifs of GGSG (SEQ ID NO: 207), e.g., GGSG (SEQ ID NO: 207). GGSGGGSG (SEQ ID NO: 217), GGSGGGSGGGSG (SEQ ID NO: 218). In some embodiments, the peptide linker comprises repeating motifs of (GGGGS)n (SEQ ID NO: 219), wherein n is an integer from 1 to 10. In some embodiments, the peptide linker does not comprise a motif. Other exemplary peptide linkers that can be used herein include, but are not limited to, GG. GGGS (SEQ ID NO: 223), PAS (SEQ ID NO:220), A(EAAAK)n (SEQ ID NO: 221), wherein n is an integer from 1 to 10, or (XP)n (SEQ ID NO: 222) wherein X is any amino acid, preferably Ala. Lys, or Glu and n is an integer from 1 to 10.

### E. Antibody

The activatable antibodies described herein comprise an antibody that is capable of specifically binding to one or more targets on a diseased cell (such as tumor) when the antigen-binding sites are not blocked by the association of the polypeptide shields (i.e., the steric shield). The antibodies contemplated herein include any suitable antibody formats having a heavy chain and a light chain, including antigen-binding fragments and antibody-drug conjugates.

In some embodiments, the antibody is a single-domain antibody fragment, such as Fv or Fab. In some embodiments, the antibody comprises two or more (such as any of 2, 3, 4, 5, or more) antigen binding domains. In some embodiments, each of the antigen binding domains are fused to polypeptide shield moieties. In some embodiments, polypeptide shield moieties are fused to the N-terminal of VH and VL where one or more (e.g., 1, 2, 3, 4, or 5) amino acids at the N-terminal of the native VH and VL are removed. In some embodiments, the antibody is a full length antibody. In some embodiments, the antibody described herein is a monoclonal antibody, polyclonal antibody, a human antibody, a mouse antibody, a rat antibody, a monkey antibody, humanized antibody, a chimeric antibody, a bispecific antibody, or a multispecific antibody.

In some embodiments, the antibody comprises an Fc region. In some embodiments, the antibody is an IgG. In some embodiments, the IgG is selected from the group consisting of IgG1, IgG2, IgG3, IgG4. In some embodiments, the antibody is an IgA, IgE, IgD, or IgM.

In some embodiments, the antibody has one or more amino acid substitutions compared to the native antibody. In some embodiments, the antibody has one or more amino acid additions to the N-terminus of the native heavy chain and/or the native light chain. In some embodiments, the antibody has one or more amino acid deletions in the N-terminal residues (such as any one or more of residues 1-5) of the native FR1 region in the heavy chain and/or the native light chain. In some embodiments, the antibody has one or more amino acid substitutions in the N-terminal residues (such as any one or more of residues 1-5) of the native FR1 region in the heavy chain and/or the native light chain.

In some embodiment, the antibody has one antigen binding domain (ABD) directed against a single target. In some embodiments, the antibody is monovalent. In some embodiments, the antibody is in the form of a Fab, Fab′, or an Fv fragment.

In some embodiments, the antibody has two or more antigen binding domains (ABDs), such as any one of 2, 3, 4, 5, 6, or more ABDs. In some embodiments, the ABDs specifically bind to the same target. In some embodiments, the ABDs specifically bind to the same epitope. In some embodiments, the ABDs specifically bind to different epitopes of the same target. In some embodiments, the antibody is multivalent, such as bivalent, trivalent, or tetravalent. In some embodiments, the ABDs specifically bind to different targets. In some embodiments, the antibody is multispecific, such as bispecific, trispecific, or tetraspecific.

The antibody (or the ABD) can be directed against any targets of interest that are expressed on a diseased cell or in a diseased tissue. In some embodiments, the antibody (or the ABD) is directed against a target that is over-expressed on a diseased cell (or in a diseased tissue) compared to a normal cell (or in a normal tissue). In some embodiments, the antibody (or the ABD) is directed against a target that is expressed at a substantially similar level on a diseased cell (or in a diseased tissue) compared to a normal cell (or in a normal tissue). In some embodiments, the antibody (or the ABD) is directed to a target expressed on a tumor cell. In some embodiments, the antibody (or the ABD) is directed to a target expressed in a tumor tissue. In some embodiments, the antibody (or the ABD) is directed to a target expressed on a tumor stromal cell. In some embodiments, the antibody (or the ABD) is directed to a target expressed on a blood vessel cell in a tumor tissue. In some embodiments, the antibody (or the ABD) is directed to a tumor antigen, such as a tumor-specific antigen or a tumor-associated antigen.

Suitable targets of the antibody (or the ABD) include, but are not limited to the molecules listed in Table 8 below. In some embodiments, the antibody (or the ABD) is capable of binding to EGFR. CTLA-4, PD-1, CD-71 or PD-L1.

TABLE 8
Exemplary targets of the activatable antibody.

| | | | | | |
|---|---|---|---|---|---|
| a-4 integrin | CD22 | CD125 | CXCR4 | GAL3STI | IgE Receptor |
| | | | | | (Fce RI) |
| a-V integrin | CD24 | CD132 | CYR61 | G-CSF | IGF |
| | | (IL-2RG) | | | |
| a-4-b-1 | CD25 | CD133 | DL44 | G-CSFR | IGF1R |
| integrin | | | | | |
| a-4-b-7 | CD27 | CD137 | DLK1 | GD2 | IL1B |
| integrin | | | | | |
| AGR2 | CD28 | CD138 | DLL4 | GITR | IL1R |
| Anti- | CD30 | CD166 | DPP-4 | GLUT1 | IL2 |
| Lewis Y | | | | | |
| Apelin J | CD40 | CD172A | DSG1 | GLUT4 | IL2R |
| receptor | | | | | |
| APRIL | CD40L | CD248 | EGFR | GM-CSF | IL4 |
| B7-H3 | CD41 | CDH6 | EGFRVIII | GM-CSFR | IL4R |
| B7-H4 | CD44 | CEACM5 | Endothelin | GPIIb/IIIa | IL6 |
| | | (CEA) | b receptor | receptor | |
| | | | (ETBR) | | |
| BCMA | CD44v6 | CEACAM6 | ENPP3 | Gp130 | IL6R |
| | | (NCA-90) | | | |
| BTLA | CD47 | CLAUDIN-3 | EPCAM | GPIIB/IIIA | IL11 |
| C5 | CD51 | CLAUDIN-4 | EPHA2 | GPNMB | IL12 |
| complement | | | | | |
| C-242 | CD52 | cMet | EPHB2 | GRP78 | IL12p40 |
| CA9 | CD56 | Collagen | ERBB3 | HER-2/neu | IL12R |
| CA19-9 | CD64 | Cripto | F protein of | HGF | IL12Rb1 |
| (Lewis a) | | | RSV | | |
| Carbonic | CD70 | CSFR | FAP | hGH | IL13 |
| anhydrase 9 | | | | | |
| CD2 | CD71 | CSFR-1 | FGF-2 | HVEM | IL13R |
| CD3 | CD74 | CTLA-4 | FGF-8 | Hyaluronidase | IL15 |
| CD6 | CD80 | CTFG | FGFR-1 | ICOS | IL17 |
| CD9 | CD81 | CXCL10 | FGFR-2 | INFa | IL18 |
| CD11a | CD86 | CXCL13 | FGFR-3 | INFb | IL21 |
| CD19 | CD95 | CXCR1 | FGFR-4 | INFg | IL23 |
| CD20 | CD117 | CXCR2 | Folate | IgE | IL23R |
| | | | receptor | | |
| IL27 | MCSP | OX-40L | RAGE | TLR9 | VEGF-B |
| IL27R | Mesothelin | OX-40R | SLC44A4 | TMEM31 | VEGF-C |
| (wsx1) | | | | | |
| IL29 | MRP4 | PAR2 | TNFa | Sphingosine 1 | VEGF-D |
| | | | | Phosphate | |
| IL31 | MUC1 | PDGF-AA | STEAP1 | TNFR | VEGFR1 |
| IL31R | Mucin-16 | PDGF-BB | STEAP2 | TNFRS12A | VEGFR2 |
| | (CA-125) | | | | |
| Insulin | Na/K ATPase | PDGFRa | TAG-72 | TRAIL-R1 | VEGFR3 |
| Receptor | | | | | |
| Jagged-1 | Neutrophil | PDGFRb | TAPA1 | TRAIl-R2 | VISTA |
| | elastase | | | | |
| Jagged-2 | NGF | PD-1 | TGFb | Transferrin | WISP-1 |
| KIR | Nicastrin | PD-L1 | TIGIT | Transferrin | WISP-1 |
| | | | | Receptor | |
| LAG-3 | NOTCH1 | PD-L2 | TIM3 | TRK-A | WISP-2 |
| LIF-R | NOTHC2 | Phospahtidyl- | TLR2 | TRK B | WISP-3 |
| | | serine | | | |
| Lewis X | NOTHC3 | P1GF | TLR4 | uPAR | |
| LIGHT | NOTCH4 | PSCA | TLR6 | VCAM-1 | |
| LRP4 | NOV | PSMA | TLR7 | VEGF | |
| LRRC26 | OSM-R | RAAG12 | TLR8 | VEGF-A | |

In some embodiments, the antibody is a therapeutic antibody. In some embodiments, the antibody is derived from one or more known therapeutic antibodies known in the art. Exemplary, suitable therapeutic antibodies include, but are not limited to the antibodies listed in Table 9 below. In some embodiments, the activatable antibody comprises an antibody derived from Cetuximab, Trastuzumab. Tremelimumab. Ipilimumab. Nivolumab, Pembrolizumab, or Atezolizumab.

In some embodiments, the activatable antibody comprises an ADB comprising a heavy chain variable domain (VH) comprising three CDRs of the amino acid sequence of SEQ ID NO: 228, and a light chain variable domain (VL) comprising three CDRs of the amino acid sequence of SEQ ID NO: 229. In some embodiments, the activatable antibody comprises an ADB comprising a VH comprising an amino acid sequence having at least about 90%, 91%, 92%, 93%, 94%, 95%, 96%, 97%, 98%, 99%, or 100% sequence identity to the amino acid sequence of SEQ ID NO. 228, and a VL comprising an amino acid sequence having at least about 90%, 91%, 92%, 93%, 94%, 95%, 96%, 97%, 98%, 99%, or 100% sequence identity to the amino acid sequence of SEQ ID NO: 229 in some embodiments, the activatable antibody comprises an antibody derived from Pembrolizumab.

In some embodiments, the activatable antibody comprises an ADB comprising a heavy chain variable domain (VH) comprising three CDRs of the amino acid sequence of SEQ ID NO: 230, and a light chain variable domain (VL) comprising three CDRs of the amino acid sequence of SEQ ID NO: 231 In some embodiments, the activatable antibody comprises an ADB comprising a VH comprising an amino acid sequence having at least about 90%, 91%, 92%, 93%, 94%, 95%, 96%, 97%, 98%, 99%, or 100% sequence identity to the amino acid sequence of SEQ ID NO: 230, and a V L comprising an amino acid sequence having at least about 90%, 91%, 92%, 93%, 94%, 95%, 96%, 97%, 98%, 99%, or 100% sequence identity to the amino acid sequence of SEQ ID NO: 231. In some embodiments, the activatable antibody comprises an antibody derived from Cetuximab.

In some embodiments, the activatable antibody comprises an ADB comprising a heavy chain variable domain (VH) comprising three CDRs of the amino acid sequence of SEQ ID NO: 232, and a light chain variable domain (VL) comprising three CDRs of the amino acid sequence of SEQ ID NO 233. In some embodiments, the activatable antibody comprises an ADB comprising a V H comprising an amino acid sequence having at least about 90%, 91%, 92%, 93%, 94%, 95%, 96%, 97%, 98%, 99%, or 100% sequence identity to the amino acid sequence of SEQ ID NO: 232, and a VL comprising an amino acid sequence having at least about 90%, 91%, 92%, 93%, 94%, 95%, 96%, 97%, 98%, 99%, or 100% sequence identity to the amino acid sequence of SEQ ID NO: 233. In some embodiments, the activatable antibody comprises an antibody derived from Tremelimumab.

In some embodiments, the activatable antibody comprises an ADB comprising a heavy chain variable domain (VH) comprising three CDRs of the amino acid sequence of SEQ ID NO: 234, and a light chain variable domain (VL) comprising three CDRs of the amino acid sequence of SEQ ID NO 235. In some embodiments, the activatable antibody comprises an ADB comprising a VH comprising an amino acid sequence having at least about 90%, 91%, 92%, 93%, 94%, 95%, 96%, 97%, 98%, 99%, or 100% sequence identity to the amino acid sequence of SEQ ID NO: 234, and a VL comprising an amino acid sequence having at least about 90%, 91%, 92%, 93%, 94%, 95%, 96%, 97%, 98%, 99%, or 100% sequence identity to the amino acid sequence of SEQ ID NO: 235. In some embodiments, the activatable antibody comprises an antibody derived from Trastuzumab.

In some embodiments, the activatable antibody comprises an ADB comprising a heavy chain variable domain (VH) comprising three CDRs of the amino acid sequence of SEQ ID NO-236, and a light chain variable domain (V L) comprising three CDRs of the amino acid sequence of SEQ ID NO: 237. In some embodiments, the activatable antibody comprises an ADB comprising a V H comprising an amino acid sequence having at least about 90%, 91%, 92%, 93%, 94%, 95%, 96%, 97%, 98%, 99%, or 100% sequence identity to the amino acid sequence of SEQ ID NO. 236, and a VL comprising an amino acid sequence having at least about 90%, 91%, 92%, 93%, 94%, 95%, 96%, 97%, 98%, 99%, or 100% sequence identity to the amino acid sequence of SEQ ID NO: 237. In some embodiments, the activatable antibody comprises an antibody derived from Ipilimumab.

In some embodiments, the activatable antibody comprises an ADB comprising a heavy chain variable domain (VH) comprising three CDRs of the amino acid sequence of SEQ ID NO: 238, and a light chain variable domain (VL) comprising three CDRs of the amino acid sequence of SEQ ID NO. 239. In some embodiments, the activatable antibody comprises an ADB comprising a VH comprising an amino acid sequence having at least about 90%, 91%, 92%, 93%, 94%, 95%, 96%, 97%, 98%, 99%, or 100% sequence identity to the amino acid sequence of SEQ ID NO: 238, and a VL comprising an amino acid sequence having at least about 90%, 91%, 92%, 93%, 94%, 95%, 96%, 97%, 98%, 99%, or 100% sequence identity to the amino acid sequence of SEQ ID NO: 239. In some embodiments, the activatable antibody comprises an antibody derived from 9D9.

In some embodiments, the antibody comprises a heavy chain variable domain (VH) sequence having at least 90%, 91%, 92%, 93%, 94%, 95%, 96%, 97%, 98%, 99%, or 100% sequence identity to the amino acid sequence of the VH of an antibody of Table 9. In some embodiments, the antibody comprises a light chain variable domain (VL) sequence having at least 90%, 91%, 92%, 93%, 94%, 95%, 96%, 97%, 98%, 99%, or 100% sequence identity to the amino acid sequence of the V L of an antibody of Table 9. In some embodiments, a VH or VL sequence having at least 90%, 91%, 92%, 93%, 94%, 95%, 96%, 97%, 98%, or 99% identity contains substitutions (e.g., conservative substitutions), insertions, or deletions relative to the reference sequence, but the antibody comprising that sequence retains the ability to bind to its target. In some embodiments, substitutions, insertions, or deletions occur in regions outside the HVRs (i.e., in the FRs).

TABLE 9
Exemplary therapeutic antibodies or antibody-drug conjugates.

| Antibody | Target | Antibody | Target |
|---|---|---|---|
| Atezoliaumab (e.g., | PD-L1 | Duravalumab | PD-L1 |
| TECENTRIQ ®) | | | |
| Bevacizumab (e.g., AVASTIN ®) | VEGF-A | Ipilimumab | CTLA-4 |
| | | (e.g., YERVOY ®) | |
| Ranibizumab (e.g., LUCENTIS ®) | VEGF | Tremelimumab | CTLA-4 |
| Cetuximab (e.g., ERBITUX ®) | EGFR | Nivolumab (e.g., | PD1 |
| | | OPDIVO ®) | |
| Panitumumab (e.g., | EGFR | Pembrolizumab(e.g., | PD-1 |
| VECTIBIX ®) | | KEYTRUDA ®) | |
| Infliximab (e.g., REMICADE ®) | TNFa | Utomilumab (PF- | CD137 |
| | | 05082566) | |
| Adalimumab (e.g., HUMIRA ®) | TNFa | Hu5F9-G4 | CD47 |
| Natalizumab (e.g., TYSABRI ®) | Integrina4 | Ertumaxomab | CD3/HER-2-neu |
| Basiliximab (e.g., SIMULECT ®) | IL2R | Tanezumab | NGF |
| Eculizumab (e.g., SOLIRIS ®) | Complement CS | Bavituximab | Phosphatidylserine |
| Efalizumab (e.g., RAPTIVA ®) | CD11a | Zalutumumab | EGFR |
| Tositumomab (e.g., BEXXAR ®) | CD20 | Matuzumab | EGFR |
| Ibritumomab tiuxetan (e.g., | CD20 | Nimotuzumab | EGFR |
| ZEVALIN ®) | | | |
| Rituximab (e.g., RITUXAN ®) | CD20 | Figitumumab | IGF1R |
| Ocrelizumab (e.g., OCREVUS ®) | CD20 | Tocilizumab (e.g., | IL-6R |
| | | ACTEMRA ®) | |
| Ofatumumab (e.g., ARZERRA ®) | CD20 | Mapatumumab | TRAIL-R1 |
| | | (HGS-ETR1) | |
| Obinutuzumab (e.g., | CD20 | Teprotumumab | IGF-1R |
| GAZYVA ®) | | | |
| Daclizumab (e.g., ZENAPAX ®) | CD25 | MDX447 | EGFR/CD64 |
| Brentuximab vedotin (e.g., | CD30 | HuJ591 | PSMA |
| ADCENTRIS ®) | | | |
| Gemtuzumab | CD33 | Denosumab (e.g., | RANKL |
| | | PROLIA ®) | |
| Gemtuzumab ozogamicin (e.g., | CD33 | Ustekinumab (e.g., | Il-12/IL-23 |
| MYELOTARG ®) | | STELARA ®) | |
| Alemtuzumab (e.g., | CD52 | Adecatumumab | EpCAM |
| CAMPATH ®) | | | |
| Abiciximab (e.g., REOPRO ®) | Glycoprotein | Abrilumab | integrin α4β7 |
| | receptor IIb/IIIa | | |
| Omalizumab (e.g., XOLAIR ®) | IgE | Belimumab | BAFF |
| Trastuzumab (e.g., | HER-2 | Briakinumab | IL-12; IL-23 |
| HERCEPTIN ®) | | | |
| Trastuzumab emtansine (e.g., | HER-2 | Lexatumumab | TRAIL-R2 |
| KADCYLA ®) | | | |
| Palivizumab (e.g., SYNAGIS ®) | F protein of RSV | Oxelumab | OX-40 |
| Ruplizumab (Hu5c8) | CD40L | Ziralimumab | CD147 |
| Pertuzumab | HER-2/neu | Inotuzumab | CD22 |
| | | ozogamicin | |
| Avelumab | PD-L1 | | |

### F. Binding Affinity

Binding affinity and specificity of the activatable antibodies described herein can be determined experimentally by methods known in the art. Such methods comprise, but are not limited to Western blots. ELISA, RIA, ECL, IRMA, EIA, and BIACORE® based binding assays and peptide scans.

In some embodiments, the K_{D} of the binding between the activatable antibody having intact S1 and S2 associated with each other and the target of the ABD is no stronger than about any one of 10⁻⁹ M, 10⁻⁸ M, 10⁻⁷ M, 10⁻⁶ M, 10⁻⁵ M, 10⁻⁴ M, 10⁻³ M, or less. In some embodiments, the K_{D} of the binding between the activatable antibody after disruption (e.g., cleavage) of the DS1 and/or DS2 and the target of the ABD is stronger than about any one of 10⁻⁷ M, 10⁻⁸ M, 10⁻⁹ M, 10⁻¹⁰ M, 10⁻¹¹ M, or 10⁻¹² M. In some embodiments, the K_{D} of the binding between the activatable antibody and the target of the ABD after disruption (e.g., cleavage) of the DS1 and/or DS2 is at least about any one of 2×, 5×, 10×, 20×, 50×, 100×, 200×, 500×, 1000× or stronger than the K_{D} of the binding between the activatable antibody having intact S1 and S2 associated with each other and the target of the ABD. In some embodiments, the K_{D} of the binding between the ABD and the target of the ABD is at least about any one of 2×, 5×, 10×, 20×, 50×, 100×, 200×, 500×, 1000× or stronger than the K_{D} of the binding between the activatable antibody having intact S1 and S2 associated with each other and the target of the ABD.

In some embodiments, the Kₒₙ of the binding between activatable antibody having S1 and S2 associated with each other and the target of the ABD is no more than about any one of 1 M⁻¹s⁻¹, 10 M⁻¹s⁻¹, 100 M⁻¹s⁻¹, 10³ M⁻¹s⁻¹, 10⁴ M⁻¹s⁻¹, 10⁵ M⁻¹s⁻¹, or 10⁶ M⁻¹s⁻¹.

In some embodiments, the K_{off} of the binding between activatable antibody having S1 and S2 associated with each other and the target of the ABD is at least about any one of 10³ s⁻¹, 100 s⁻¹, 10 s⁻¹, 1 s⁻¹, 10⁻² s⁻¹, 10⁻³ s⁻¹.

In some embodiments, the EC₅₀ of the activatable antibody having intact S1 and S2 associated with each other in a cell proliferation assay with a target cell expressing the target of the ABD is more than about any one of 2×, 5×, 10×, 20×, 30×, 40×, 50×, 100×, 200×, 500×, 1000× or more than that of the EC₅₀ of the activatable antibody after disruption (e.g., cleavage) of the DS1 and/or DS2 in the same cell proliferation assay.

### III. Methods of Preparing Activatable Antibodies

Further provided are methods of preparing the activatable antibodies described herein. As the poly peptide shield moieties (i.e., S1 and S2) of the activatable antibodies are genetically fused to the antibody component in the activatable antibodies, any of the known antibody expression and purification methods in the art can be used to prepare a fusion antibody comprising the polypeptide shield moieties fused to the N-termini of the VH and VL. In some embodiments, an activatable antibody is obtained while the polypeptide shield moieties in the fusion antibody associate with each other during the antibody expression process. In some embodiments, the method further comprises treating the fusion antibody under a condition that allows association of the polypeptide shield moieties, thereby providing the activatable antibody. “Heavy chain” when used in conjunction with the activatable antibody refers to the polypeptide chain comprising the polypeptide shield moiety S1 fused to the N-termini of the VH of the antibody. “Light chain”, when used in conjunction with the activatable antibody refers to the polypeptide chain comprising the polypeptide shield moiety S2 fused to the N-termini of the VL of the antibody. “Fusion antibody” refers to the protein complex formed by the heavy chain(s) and the light chain(s) of the activatable antibody, wherein the steric shield may or may not be formed.

### A. Nucleic Acid Molecules Encoding Activatable Antibodies

Nucleic acid molecules comprising polynucleotides that encode one or more chains of the activatable antibodies are provided. In some embodiments, there is provided a first nucleic acid encoding a heavy chain comprising S1 fused to the N-terminus of the VH in the activatable antibody, and a second nucleic acid encoding a light chain comprising S2 fused to the N-terminus of the VL in the activatable antibody. In some embodiments, there is provided a nucleic acid encoding a heavy chain comprising S1 fused to the N-terminus of the VH in the activatable antibody, and a second nucleic acid encoding a light chain comprising S2 fused to the N-terminus of the VL in the activatable antibody. In some embodiments, wherein the activatable antibody comprises two non-identical heavy chains and two non-identical light chains, a nucleic acid encoding each of the four chains or a single nucleic acid encoding all four chains is provided. In some embodiments, the nucleic acids encoding the heavy chain(s) and the light chain(s) are operably linked to the same promoter. In some embodiments, the nucleic acids encoding the heavy chain(s) and the light chain(s) are operably linked to different promoters.

In some embodiments, a nucleic acid encoding a heavy chain or light chain of the activatable antibody comprises a nucleotide sequence that encodes a leader sequence, which, when translated, is located at the N terminus of the heavy chain or light chain. The leader sequence may be the native antibody heavy or light chain leader sequence, or may be another heterologous leader sequence.

Nucleic acid molecules may be constructed using recombinant DNA techniques conventional in the art. In some embodiments, a nucleic acid molecule is an expression vector that is suitable for expression in a selected host cell. Vectors comprising polynucleotide(s) that encode the heavy chains and light chain(s) of the activatable antibodies are provided. Such vectors include, but are not limited to, DNA vectors, phage vectors, viral vectors, retroviral vectors, etc. In some embodiments, there is provided a vector comprising: (a) a first nucleic acid encoding a heavy chain comprising S1 fused to the N-terminus of the VH, and (b) a second nucleic acid encoding a light chain comprising S2 fused to the N-terminus of the VL.

In some embodiments, a first vector comprises a first nucleic acid encoding the heavy chain of the activatable antibody and a second vector comprises a second nucleic acid encoding the light chain of the activatable antibody. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1.1 and 1:5 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, e.g., in Running Deer et al., Biotechnol. Prig. 20:880-889 (2004).

### B. Host Cells

In various embodiments, the heavy chains and/or light chains of the activatable antibodies may be expressed in prokaryotic cells, such as bacterial cells; or in eukaryotic cells, such as fungal cells, yeast cells, plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44, Lec13 CHO cells, and FUT8 CHO cells; PER C6® cells (Crucell); and NSO cells. In some embodiments, a particular eukaryotic host cell is selected based on its ability to make desired post-translational modifications to the activatable antibodies. In some embodiments, a host cell capable of protein glycosylation is provided. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Non-limiting exemplary methods are described. e.g., in Sambrook et al., Molecular Cloning. A Laboratory Manual, 3^{rd} ed. Cold Spring Harbor Laboratory Press (2001). Nucleic acids may be transiently or stably transfected in the desired host cells, according to any suitable method.

The invention also provides host cells comprising any of the nucleic acids or vectors described herein. In some embodiments, the invention provides a host cell comprising an activatable antibody or a vector encoding the heavy chain(s) and/or light chain(s) of the activatable antibody. Any host cells capable of over-expressing heterologous DNAs can be used for the purpose of isolating the genes encoding the activatable antibody, polypeptide or the corresponding fusion antibodies. Non-limiting examples of mammalian host cells include but not limited to COS, HeLa, and CHO cells. See also PCT Publication No. WO 87/04462. Suitable non-mammalian host cells include prokaryotes (such as E. coli or B. subtilis) and yeast (such as S. cerevisae. S pombe; or K. lactis).

In some embodiments, the activatable antibody is produced in a cell-free system. Non-limiting exemplary cell-free systems are described. e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### C. Purification

The activatable antibodies or the corresponding fusion proteins may be purified by any suitable method. Such methods include, but are not limited to, the use of affinity matrices or hydrophobic interaction chromatography. Suitable affinity ligands include the ROR1 ECD and ligands that bind antibody constant regions. For example, a Protein A. Protein G, Protein A/G, or an antibody affinity column may be used to bind the constant region and to purify, a full-length activatable antibody. Hydrophobic interactive chromatography, for example, a butyl or phenyl column, may also suitable for purifying some activatable antibodies. Ion exchange chromatography (e.g. anion exchange chromatography and/or cation exchange chromatography) may also suitable for purifying some activatable antibodies. Mixed-mode chromatography (e.g. reversed phase/anion exchange, reversed phase/cation exchange, hydrophilic interaction/anion exchange, hydrophilic interaction/cation exchange, etc.) may also suitable for purifying some activatable antibodies. Many methods of purifying polypeptides are known in the art and may be used to purify the fusion antibody prior to the formation of the steric shield, and/or the activatable antibody after the formation of the steric shield.

### D. Formation of Steric Shield

In some embodiments, the steric shield forms, i.e., S1 and S2 associates with each other spontaneously upon expression of the fusion antibody corresponding to the activatable antibody. In some embodiments, there is provided a method of preparing an activatable antibody, comprising: (a) culturing a host cell comprising one or two vectors encoding the heavy chain(s) and the light chain(s) of the activatable antibody under a condition that produces a fusion antibody comprising the heavy chain(s) and the light chain(s), and (b) isolating the fusion antibody, thereby providing the activatable antibody.

In some embodiments, the fusion antibody is subject to an enzymatic or chemical treatment step to conjugate S1 and S2. In some embodiments, the fusion antibody is subject to a chemical treatment step to reform the covalent bond between S1 and S2. In some embodiments, S1 and S2 are conjugated to each other via one or more disulfide bonds.

In some embodiments, there is provided a method of preparing an activatable antibody, wherein S1 and S2 comprise cysteines, and S1 and S2 are conjugated to each other via one or more disulfide bonds, the method comprising (a) culturing a host cell comprising one or two vectors encoding the heavy chain(s) and the light chain(s) of the activatable antibody under a condition that produces a fusion antibody comprising the heavy chain(s) and the light chain(s), (b) isolating the fusion antibody; and (c) treating the fusion antibody with a reducing agent followed by an oxidation agent, thereby providing the activatable antibody. In some embodiments, the reducing agent is TCEP and the oxidation agent is ascorbic acid. In some embodiments, the molar ratio between the fusion antibody to the TCEP is at least about any one of 10:1, 5:1, 4:1, 3:1, 2:1, 1.1, 1:2, or 1:5. In some embodiments, the molar ratio between the TCEP to ascorbic acid is no more than about any one of 1:1, 1:2, 1:3, 1:4, 1:5, 1:10 or more. In some embodiments, the reducing agent is reduced glutathione, and the oxidation agent is oxidized glutathione. Any other known reducing agents and oxidization agents suitable for protein preparation can also be used.

In some embodiments, there is provided a method of preparing an activatable antibody, wherein one of S1 and S2 comprises an acyl donor residue and the other comprises an acyl acceptor residue, and wherein S1 and S2 are conjugated to each other by a transglutaminase, the method comprising: (a) culturing a host cell comprising a vector encoding the heavy chain(s) and the light chain(s) of the activatable antibody under a condition that produces a fusion antibody comprising the heavy chain(s) and the light chain(s); (b) isolating the fusion antibody; and (c) treating the fusion antibody with the transglutaminase, thereby providing the activatable antibody.

In some embodiments, there is provided a method of preparing an activatable antibody, wherein one of S1 and S2 comprises a glycan-binding protein (such as lectin, ficolin, or ERGIC-53) and the other comprises an glycan attached to a glycosylation residue, the method comprising: (a) culturing a host cell comprising a vector encoding the heavy chain(s) and the light chain(s) of the activatable antibody under a condition that produces a fusion antibody comprising the heavy chain(s) and the light chain(s); (b) isolating the fusion antibody; and (c) treating the fusion antibody with a glycosylase, thereby providing the activatable antibody.

Also provided is a composition comprising the fusion antibody prepared in any one of the methods described above.

### IV. Methods of Treatment

The activatable antibodies described herein and compositions thereof (such as pharmaceutical composition) can be used to treat various diseases, such as cancer, including solid tumor or hematological cancer. Thus, in some embodiments, there is provided a method of treating a disease in an individual (such as a human), comprising administering to the individual an effective amount of any one of the activatable antibodies described herein. In some embodiments, the disease is cancer. In some embodiments, the cancer is selected from the group consisting of bladder cancer, brain cancer, breast cancer, cervical cancer, colorectal cancer, esophageal cancer, kidney cancer, leukemia, liver cancer, wig cancer, melanoma, non-Hodgkin's lymphoma, ovarian cancer, pancreatic cancer, prostate cancer, skin cancer, thyroid cancer and uterine cancer.

In some embodiments, there is provided a method of treating a disease (such as cancer) in an individual, comprising administering to the individual an effective amount of an activatable antibody comprising a therapeutic antibody comprising an antigen-binding domain (ABD), w-herein the ABD comprises a heavy chain variable region (VH) and a light chain variable region (VL), wherein the N-terminus of the VH is fused to a first polypeptide shield moiety (S1), and the N-terminus of the VL is fused to a second polypeptide shield moiety (S2), wherein S1 comprises a first disease-sensing releasable moiety (DS1) and/or S2 comprises a second disease-sensing releasable moiety (DS2), wherein association of S1 With S2 blocks binding of the ABD to its target, wherein the ABD does not specifically bind to S1. S2, or association thereof, and wherein DS1 and/or DS2 are disrupted (such as cleaved) at a diseased site, thereby unblocking binding of the ABD to its target at the diseased site. In some embodiments, S1 comprises a first association moiety B, and S2 comprises a second association moiety C, wherein B and C associate with each other covalently and/or non-covalently. In some embodiments, DS1 and/or DS2 comprises a peptide substrates specific for an enzyme (e.g., protease) that is expressed at a diseased site. In some embodiments, DS1 and/or DS2 comprise a pH-sensitive protein or peptide. In some embodiments, the disease is a cancer. In some embodiments, the cancer is selected from the group consisting of bladder cancer, brain cancer, breast cancer, cervical cancer, colorectal cancer, esophageal cancer, kidney cancer, leukemia, liver cancer, lung cancer, melanoma, non-Hodgkin's lymphoma, ovarian cancer, pancreatic cancer, prostate cancer, skin cancer, thyroid cancer and uterine cancer.

In some embodiments, there is provided a method of preferentially delivering a therapeutic antibody to a diseased site in an individual in need of treatment with the therapeutic antibody, comprising administering to the individual an effective amount of an activatable antibody comprising the therapeutic antibody comprising an antigen-binding domain (ABD), wherein the ABD comprises a heavy chain variable region (VH) and a light chain variable region (VL), wherein the N-terminus of the VH is fused to a first polypeptide shield moiety (S1), and the N-terminus of the VL is fused to a second polypeptide shield moiety (S2), wherein S1 comprises a first disease-sensing releasable moiety (DS1) and/or S2 comprises a second disease-sensing releasable moiety-(DS2), wherein association of S1 with S2 blocks binding of the ABD to its target, wherein the ABD does not specifically bind to S1, S2, or association thereof and wherein DS1 and/or DS2 are disrupted (such as cleaved) at a diseased site, thereby unblocking binding of the ABD to its target at the diseased site. In some embodiments, S1 comprises a first association moiety B, and S2 comprises a second association moiety C, wherein B and C associate with each other covalently and/or non-covalently. In some embodiments, DS1 and/or DS2 comprises a peptide substrates specific for an enzyme (e.g., protease) that is expressed at a diseased site. In some embodiments, DS1 and/or DS2 comprise a pH-sensitive protein or peptide. In some embodiments, the disease is a cancer. In some embodiments, the cancer is selected from the group consisting of bladder cancer, brain cancer, breast cancer, cervical cancer, colorectal cancer, esophageal cancer, kidney cancer, leukemia, liver cancer, lung cancer, melanoma, non-Hodgkin's lymphoma, ovarian cancer, pancreatic cancer, prostate cancer, skin cancer, thyroid cancer and uterine cancer.

In some embodiments, there is provided a method of increasing the effective concentration of a therapeutic antibody at a diseased site in an individual in need of treatment with the therapeutic antibody, comprising administering to the individual an effective amount of an activatable antibody comprising the therapeutic antibody comprising an antigen-binding domain (ABD), wherein the ABD comprises a heavy chain variable region (VH) and a light chain variable region (VL), wherein the N-terminus of the VH is fused to a first polypeptide shield moiety (S1), and the N-terminus of the VL is fused to a second polypeptide shield moiety (S2), wherein S1 comprises a first disease-sensing releasable moiety (DS1) and/or S2 comprises a second disease-sensing releasable moiety (DS2), wherein association of S1 with S2 blocks binding of the ABD to its target, wherein the ABD does not specifically bind to S1, S2, or association thereof, and wherein DS1 and/or DS2 are disrupted (such as cleaved) at a diseased site, thereby unblocking binding of the ABD to its target at the diseased site. In some embodiments. S1 comprises a first association moiety B, and S2 comprises a second association moiety C, wherein B and C associate with each other covalently and/or non-covalently. In some embodiments, DS1 and/or DS2 comprises a peptide substrates specific for an enzyme (e.g., protease) that is expressed at a diseased site. In some embodiments, DS1 and/or DS2 comprises a pH-sensitive protein or peptide. In some embodiments, the disease is a cancer. In some embodiments, the cancer is selected from the group consisting of bladder cancer, brain cancer, breast cancer, cervical cancer, colorectal cancer, esophageal cancer, kidney cancer, leukemia, liver cancer, lung cancer, melanoma, non-Hodgkin's lymphoma, ovarian cancer, pancreatic cancer, prostate cancer, skin cancer, thyroid cancer and uterine cancer. In some embodiments, the effective concentration of the therapeutic antibody is increased by about any of 10%, 20%, 50%, 1×, 2×, 5× or more at the diseased site compared to administration of the therapeutic antibody to the individual at the same effective amount.

In some embodiments, there is provided a method of reducing binding of a therapeutic antibody to a target in normal tissues in an individual having a disease in need of treatment with the therapeutic antibody, comprising administering to the individual an effective amount of an activatable antibody comprising the therapeutic antibody comprising an antigen-binding domain (ABD), wherein the ABD comprises a heavy chain variable region (VH) and a light chain variable region (VL), wherein the N-terminus of the VH is fused to a first polypeptide shield moiety (S1), and the N-terminus of the VL is fused to a second polypeptide shield moiety (S2), wherein S1 comprises a first disease-sensing releasable moiety (DS1) and/or S2 comprises a second disease-sensing releasable moiety (DS2), wherein association of S1 with S2 blocks binding of the ABD to its target, wherein the ABD does not specifically bind to S1, S2, or association thereof, and wherein DS1 and/or DS2 are disrupted (such as cleaved) at a diseased site, thereby unblocking binding of the ABD to its target at the diseased site. In some embodiments, S1 comprises a first association moiety B, and S2 comprises a second association moiety C, wherein B and C associate with each other covalently and/or non-covalently. In some embodiments, DS1 and/or DS2 comprises a peptide substrates specific for an enzyme (e.g., protease) that is expressed at a diseased site. In some embodiments, DS1 and/or DS2 comprises a pH-sensitive protein or peptide. In some embodiments, the disease is a cancer. In some embodiments, the cancer is selected from the group consisting of bladder cancer, brain cancer, breast cancer, cervical cancer, colorectal cancer, esophageal cancer, kidney cancer, leukemia, liver cancer, lung cancer, melanoma, non-Hodgkin's lymphoma, ovarian cancer, pancreatic cancer, prostate cancer, skin cancer, thyroid cancer and uterine cancer. In some embodiments, administration of the activatable antibody reduces binding of the therapeutic antibody to its target on normal cells by about any of 10%, 20%, 30%, 40%, 50%, 60%, 70%, 80%, 90% or more compared to administration of the therapeutic antibody to the individual.

In some embodiments, there is provided a method of reducing toxicity (such as on-target off-tissue toxicity) of a therapeutic antibody to an individual having a disease in need of treatment with the therapeutic antibody, comprising administering to the individual an effective amount of an activatable antibody comprising the therapeutic antibody comprising an antigen-binding domain (ABD), wherein the ABD comprises a heavy chain variable region (VH) and a light chain variable region (VU, wherein the N-terminus of the VH is fused to a first polypeptide shield moiety (S1), and the N-terminus of the VL is fused to a second polypeptide shield moiety (S2), wherein S1 comprises a first disease-sensing releasable moiety (DS1) and/or S2 comprises a second disease-sensing releasable moiety (DS2), wherein association of S1 with S2 blocks binding of the ABD to its target, wherein the ABD does not specifically bind to S1, S2, or association thereof, and wherein DS1 and/or DS2 are disrupted (such as cleaved) at a diseased site, thereby unblocking binding of the ABD to its target at the diseased site. In some embodiments. S1 comprises a first association moiety B, and S2 comprises a second association moiety C, wherein B and C associate with each other covalently and/or non-covalently. In some embodiments, DS1 and/or DS2 comprises a peptide substrates specific for an enzyme (e.g., protease) that is expressed at a diseased site. In some embodiments, DS1 and/or DS2 comprises a pH-sensitive protein or peptide. In some embodiments, the disease is a cancer. In some embodiments, the cancer is selected from the group consisting of bladder cancer, brain cancer, breast cancer, cervical cancer, colorectal cancer, esophageal cancer, kidney cancer, leukemia, liver cancer, lung cancer, melanoma, non-Hodgkin's lymphoma, ovarian cancer, pancreatic cancer, prostate cancer, skin cancer, thyroid cancer and uterine cancer. In some embodiments, administration of the activatable antibody reduces the toxicity of the therapeutic antibody by about any of 10%, 20%, 30%, 40%, 50%, 60%, 70%, 80%, 90% or more compared to administration of the therapeutic antibody at the same effective amount to the individual.

The activatable antibodies described herein can be administered to an individual via various routes, for example, with the same route of administration as the therapeutic antibody that the activatable antibody is based on. Exemplary mutes of administration include, but are not limited to parenteral, intravenous, intramuscular, and subcutaneous administration.

The effective amount, suitable dose, and dosing schedule of the activatable antibody administered to an individual can vary depending on the particular composition, the route of administration, and the particular type of disease being treated. In some embodiments, the effective amount of the activatable antibody is the same as the effective amount of the therapeutic antibody. In some embodiments, the effective amount of the activatable antibody is at least about any of 90% 80%, 70%, 60%, 50%, 40%, 30%, 20%, or less than the effective amount of the therapeutic antibody.

In some embodiments, the individual is a mammal, such as human, rodents, or primates. In some embodiments, the individual is human.

### V. Pharmaceutical Compositions, Kits, and Articles of Manufacture

The present application further provides pharmaceutical compositions, kits, and articles of manufacture comprising any one of the activatable antibodies described herein. In some embodiments, there is provided a pharmaceutical composition comprising any one of the activatable antibodies described herein and a pharmaceutically-acceptable carrier. In some embodiments, the composition (such as pharmaceutical composition) further comprises a carrier, diluent, or excipient, which may facilitate administration of the composition to an individual in need thereof. Examples of carriers, diluents, and excipients include, but are not limited to, calcium carbonate, calcium phosphate, various sugars such as lactose, or types of starch, cellulose derivatives, gelatin, vegetable oils, polyethylene glycols and physiologically compatible solvents.

If desired, solutions of the above compositions may be thickened with a thickening agent such as methylcellulose. They may be prepared in emulsified form, either water in oil or oil in water. Any of a wide variety of pharmaceutically acceptable emulsifying agents may be employed including, for example, acacia powder, a non-ionic surfactant (such as a Tween), or an ionic surfactant (such as alkali polyether alcohol sulfates or sulfonates, e.g., a Triton).

The pharmaceutical compositions described herein can be prepared by mixing the ingredients following generally accepted procedures. For example, the selected components may be simply mixed in a blender or other standard device to produce a concentrated mixture which may then be adjusted to the final concentration and viscosity by the addition of water or thickening agent and possibly a buffer to control pH or an additional solute to control tonicity.

Other pharmaceutically acceptable carriers and their formulation are described in standard formulation treatises. e.g., Remington's Pharmaceutical Sciences by E. W. Martin. See also Wang, Y. J, and Hanson, M. A. “Parenteral Formulations of Proteins and Peptides Stability and Stabilizers,” Journal of Parenteral Science and Technology, Technical Report No. 10, Supp. 42:2S (1988).

In some embodiments, the pharmaceutical composition is a liquid suspension. In some embodiments, the pharmaceutical composition is a lyophilized powder. In some embodiments, the pharmaceutical composition is a sterile composition.

Also provided are kits comprising any one of the activatable antibodies described herein. The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease as described herein, and may have a sterile access port. The label or package insert indicates that the composition is used for treating the particular condition in an individual. The label or package insert will further comprise instructions for administering the composition to the individual.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a disease (such as cancer).

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The examples below are intended to be purely exemplary of the invention and should therefore not be considered to limit the invention in any way. The following examples and detailed description are offered by way of illustration and not by way of limitation Example 1. Production of fusion antibodies.

Fusion antibodies that correspond to exemplary activatable antibodies, including those comprising a fill-length antibody or Fab fragments, were prepared by expressing nucleic acids encoding the heavy chain and light chain of each activatable antibody.

Briefly, fusion gene constructs were generated using gene synthesis and codon optimized for expression in mammalian cells (DNA2.0). The heavy and light chain genes were cloned into separate mammalian expression vectors, and then co-transfected in EXPI293F™ cells (Thermo Fisher). Antibody expression was carried out in EXPI293F™ Expression Medium (Thermo Fisher). The supernatant was harvested at 5-7 days post transfection.

Fusion antibodies comprising full-length antibodies were purified using MABSELECT™ Sure resin (GE Healthcare Life Sciences) The fusion antibodies were loaded onto the resin in 20 mM sodium phosphate, 150 mM NaCl. pH 7.2 and eluted using 0.1 M sodium citrate, pH 3.5. Fractions were eluted into 1.0 M Tris-HCl, pH 8.0, pooled and buffer exchanged into 1×PBS, pH 7.4 buffer. Size-exclusion data indicated that none of the fusion antibodies was aggregated during expression (data not shown).

In fusion Fab constructs, each of the heavy chains is designed to carry a 10× histidine affinity tag, which allows purification of the fusion Fabs using Immobilized Metal Affinity Chromatography (IMAC). Briefly, HIS-TRAP™ Ni Sepharose resin column (GE Healthcare) is first equilibrated with 5 mM imidazole, 20 mM TrisHCl (pH7.4), 500 mM NaCl buffer. The clarified media expressing each fusion Fab is loaded onto HIS-TRAP™ Ni Sepharose excel affinity resin and column washed with 5 mM imidazole, 20 mM TrisHCl (pH7.4), 500 mM NaCl buffer. Then, the column is washed with 40 mM imidazole, 20 mM Tris HCl pH 7.4, 500 mM NaCl buffer and subsequently eluted with 200 mM imidazole, 20 mM Tris HCl pH 7.4 500 mM NaCl buffer. Eluted fractions containing each fusion Fab are pooled and buffer exchanged into 1×PBS buffer, pH 7.4.

### Example 2. Design of Exemplary Activatable Antibodies

This example describes the design of various exemplary activatable antibodies (including activatable antibody fragments). The activatable antibodies described herein remain inert until activated locally in a diseased tissue to bind to their antigens. This property w ill minimize binding of the activatable antibody to its antigen on the surface of non-diseased cells and prevent undesirable “on-target-off-tissue” toxicity. To generate such constructs, exemplary activatable antibodies are designed to carry a releasable steric shield covering their antigen binding sites thus preventing antigen binding Polypeptide shield moieties S1 and S2 are genetically fused, respectively, to the N-termini of the heavy chain and light chain of a therapeutic antibody. The polypeptide shield moieties internet with each other to provide the steric shield, which does not bind to the antigen binding sites. Rather, the steric shield hoovers over the antigen binding sites at such a distance as to present steric hindrance to antigen binding. A disease-sensing releasable moiety is further incorporated as a pat of each polypeptide shield moiety. In the presence of a diseased tissue-specific element (such as an enzyme), the disease-sensing releasable moieties are disrupted. Consequently, the steric shield is either allowed to freely diffuse away from the antibody, or cleaved such that it no longer joins the N-termini of the heavy chain and light chain, but portions of the polypeptide shield moieties remain attached to the antibody.

The fusion antibodies corresponding to the activatable antibodies are produced as described in Example 1, and the steric shields are formed during expression, or by treatment of the fusion antibodies with transglutaminase (transglutaminase-conjugated activatable antibodies), oxidation/reduction agents (Cys-Cys disulfide-conjugated activatable antibodies), or glycosylase (glycan-binding protein-glycan) antibodies, thereby providing the activatable antibodies. Formation of the steric shields is assessed by SBS-PAGE gel analysis. Antigen binding of the activatable antibodies in the presence and absence of the steric shields is evaluated by surface plasmon resonance as described in Example 9.

A. Exemplary Activatable Anti-EGFR Antibody Constructs with Covalently Associated Polypeptide Shield Moieties.

Four exemplary activatable anti-EGFR antibody (referred herein as “activatable EGFR Ab”) constructs were prepared. Polypeptide shield moieties were genetically fused to the N-termini of the heavy chain and the light chain of a full-length therapeutic anti-EGFR antibody comprising a VH comprising the amino acid sequence of SEQ ID NO: 230, and a VL comprising the amino acid sequence of SEQ ID NO. 231. The anti-EGFR antibody was modified to carry a point mutation that abolished its native N-glycosylation site in the Fab region to facilitate analysis. The activatable EGFR Ab constructs (and the corresponding fusion antibodies) comprise sequences that are listed in Table 10 below. In each sequence, the polypeptide shield moiety is underlined, including an association moiety (the single underlined portion with bold letters), a disease-sensing releasable moiety (the underlined portion with regular letters. SEQ ID NO: 19) which can be cleaved by disease-specific proteases, and in some cases, a flexible peptide linker (italicized letters with double underlines). Activatable EGFR Ab 1 and 2 had acyl donor (SEQ ID NO: 1) and acyl acceptor sequences (SEQ ID NO 2) to allow transglutaminase-catalyzed conjugation. A skilled person in the art would readily appreciate that other acyl donor and acyl acceptor sequences, cysteine-containing peptides, disease-sensing releasable moieties (such as protease substrate peptides) and therapeutic antibodies described herein can be used to prepare similar activatable antibodies.

The lengths of the polypeptide shield moieties were designed based on the structural models as described in Example 3. In constructs having a transglutaminase-conjugated steric shield (e.g., activatable EGFR Ab 1 and 2), each polypeptide shield was designed to have 11, 12, 15 or 16 amino acids, but other lengths are possible. The total numbers of added amino acid residues to the native N-termini of the anti-EGFR antibody n ere 12 for the heavy chain and 11 for the light chain in the activatable EGFR Ab 1; and 16 for the heavy and 15 for the light chain in the activatable EGFR Ab 2. After transglutaminase-catalyzed conjugation, the length of the steric shield is 20 and 28 amino acids respectively for the activatable EGFR Ab 1 and 2 respectively.

In constructs having a disulfide-conjugated steric shield (e.g., activatable EGFR Ab 3 and 4), each polypeptide shield was designed to have 12 or 14 amino acids, but other lengths are possible. The total numbers of added amino acid residues to the native N-termini of the anti-EGFR antibody were 12 for the heavy chain and 12 for the light chain in the activatable EGFR Ab 3; and 14 for the heavy and 14 for the light chain in the activatable EGFR Ab 4. After transglutaminase-catalyzed conjugation, the length of the steric shield is 22 and 26 amino acids respectively for the activatable EGFR Ab 3 and 4 respectively.

TABLE 10
Exemplary activatable anti-EGFR antibody constructs.

| | Chain | |
|---|---|---|
| | (SEQ | |
| No. | ID NO) | Sequence |
| 1 | Heavy | LLQGLSGRSDNHQVQLKQSGPGLVQPSQSLSITCTVSGFSLTNYGVHW |
| | (21) | VRQSPGKGLEWLGVIWSGGNTDYNTPFTSRLSINKDNSKSQVFFKMNS |
| | | LQSEDTAIYYCARALTYYDYEFAYWGQGTLVTVSAASTKGPSVFPLAP |
| | | SSKSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTHTCP |
| | | PCPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSRDELTKNQVSLTCLVK |
| | | GFYPSDIAVEWESNGQPENNYKTTPPVLDSDGSFFLYSKLTVDKSRWQ |
| | | QGNVFSCSVMHEALHNHYTQKSLSLSPG |
| | Light | GKGLSGRSDNHDILLTQSPVILSVSPGERVSFSCRASQSIGTNIHWYQQ |
| | (22) | RTNGSPRLLIKYASESISGIPSRFSGSGSGTDFTLSINSVESEDIADYYCQ |
| | | QNNNWPTTFGAGTKLELKRTVAAPSVFIFPPSDEQLKSGTASVVCLLN |
| | | NFYPREAKVQWKVDNALQSGNSQESVTEQDSKDSTYSLSSTLTLSKA |
| | | DYEKHKVYACEVTHQGLSSPVTKSFNRGEC |
| 2 | Heavy | LLQGLSGRSDNHGGGSQVQLKQSGPGLVQPSQSLSITCTVSGFSLTNY |
| | (23) | GVHWVRQSPGKGLEWLGVIWSGGNTDYNTPFTSRLSINKDNSKSQVF |
| | | FKMNSLQSEDTAIYYCARALTYYDYEFAYWGQGTLVTVSAASTKGPS |
| | | VFPLAPSSKSTSGGTAALGCLVKDYFPEPVTVSWNSGALTSGVHTFPA |
| | | VLQSSGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDVSHED |
| | | PEVKFNWYVDGVEVHNAKTKPREEQYNSTYRVVSVLTVLHQDWLNG |
| | | KEYKCKVSNKALPAPIEKTISKAKGQPREPQVYTLPPSRDELTKNQVSL |
| | | TCLVKGFYPSDIAVEWESNGQPENNYKTTPPVLDSDGSFFLYSKLTVD |
| | | KSRWQQGNVFSCSVMHEALHNHYTQKSLSLSPG |
| | Light | GKGLSGRSDNHGGGSDILLTQSPVILSVSPGERVSFSCRASQSIGTNIHW |
| | (24) | YQQRTNGSPRLLIKYASESISGIPSRFSGSGSGTDFTLSINSVESEDIADY |
| | | YCQQNNNWPTTFGAGTKLELKRTVAAPSVFIFPPSDEQLKSGTASVVC |
| | | LLNNFYPREAKVQWKVDNALQSGNSQESVTEQDSKDSTYSLSSTLTLS |
| | | KADYEKHKVYACEVTHQGLSSPVTKSFNRGEC |
| 3 | Heavy | SCLSGRSDNHGGQVQLKQSGPGLVQPSQSLSITCTVSGFSLTNYGVHW |
| | (25) | VRQSPGKGLEWLGVIWSGONTDYNTPFTSRLSINKDNSKSQVFFKMNS |
| | | LQSEDTAIVYCARALTYYDYEFAYWGQGTLVTVSAASTKGPSVFPLAP |
| | | SSKSTSGGTAALGCLVKDYFPEPVTVSWNSGALTSGVHTFPAVLQSSG |
| | | LYSLSSVVTVPSSSLGTQTYICNVNHKPSNTKVDKKVEPKSSDKTHTCP |
| | | PCPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSRDELTKNQVSLTCLVK |
| | | GFYPSDIAVEWESNGQPENNYKTTPPVLDSDGSFFLYSKLTVDKSRWQ |
| | | QGNVFSCSVMHEALHNHYTQKSLSLSPG |
| | Light | SCLSGRSDNHGGDILLTQSPVILSVSPGERVSFSCRASQSIGTNIHWYQQ |
| | (26) | RTNGSPRLLIKYASESISGIPSRFSGSGSGTDFTLSINSVESEDIADYYCQ |
| | | QNNNWPTTFGAGTKLELKRTVAAPSVFIFPPSDEQLKSGTASVVCLLN |
| | | NFYPREAKVQWKVDNALQSGNSQESVTEQDSKDSTYSLSSTLTLSKA |
| | | DYEKHKVYACEVTHQGLSSPVTKSFNRGES |
| 4 | Heavy | SCLSGRSDNHGGGSQVQLKQSGPGLVQPSQSLSITCTVSGFSLTNYGV |
| | (27) | HWVRQSPGKGLEWLGVIWSGGNTDYNTPFTSRLSINKDNSKSQVFFK |
| | | MNSLQSEDTAIYYCARALTYYDYEFAYWGQGTLVTVSAASTKGPSVF |
| | | PLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSSDKT |
| | | HTCPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGK |
| | | EYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDK |
| | | SRWQQGNVFSCSVMHEALHNHYTQKSLSLSPG |
| | Light | SCLSGRSDNHGGGSDILLTQSPVILSVSPGERVSFSCRASQSIGTNIHWY |
| | (28) | QQRTNGSPRLLIKYASESISGIPSRFSGSGSGTDFTLSINSVESEDIADYY |
| | | CQQNNNWPTTFGAGTKLELKRTVAAPSVFIFPPSDEQLKSGTASVVCL |
| | | LNNFYPREAKVQWKVDNALQSGNSQESVTEQDSKDSTYSLSSTLTLSK |
| | | ADYEKHKVYACEVTHQGLSSPVTKSFNRGES |

B. Exemplary Activatable Fabs with Covalently Associated Polypeptide Shield Moieties.

Based on structural models of Example 3, it was discovered that the same polypeptide shield moieties can be engineered into different antigen binding sites to provide steric shields. Exemplary activatable Fabs targeting human HER-2, human CTLA-4, human PD-1, and mouse CTLA-4 are designed and prepared. The activatable Fabs (and the corresponding fusion Fabs) comprise sequences that are shown in Table 11. The exemplary activatable Fabs were further designed to contain a His tag at the C-terminus of the heavy chain. Sequences corresponding to the polypeptide shield moieties S11 and S2 are underlined. A skilled person in the art would readily appreciate that other acyl donor and acyl acceptor sequences, cysteine-containing peptides, disease-sensing releasable moieties (such as protease substrate peptides) and antigen-binding fragments derived from therapeutic antibodies described herein can be used to prepare similar activatable antibodies.

TABLE 11
Exemplary activatable Fabs.

| | Chain | |
|---|---|---|
| | (SEQ | |
| Target | ID NO) | Sequence |
| Human | Heavy | LLQGLSGRSDNHGGGSEVQLVESGGGLVQPGGSLRLSCAASGFNIKDTYIH |
| HER-2 | (29) | WVRQAPGKGLEWVARIYPTNGYTRYADSVKGRFTISADTSKNTAYLQMNS |
| | | LRAEDTAVYYCSRWGGDGFYAMDYWGQGTLVTVSSASTKGPSVFPLAPSS |
| | | KSTSGGTAALGCLVKDYFPEPVTVSWNSGALTSGVHTFPAVLQSSGLYSLSS |
| | | VVTVPSSSLGTQTYICNVNHKPSNTKVDKKVEPKSC |
| | Light | GKGLSGRSDNHGGGSDIQMTQSPSSLSASVGDRVTITCRASQDVNTAVAWY |
| | (30) | QQKPGKAPKLLIYSASFLYSGVPSRFSGSRSGTDFTLTISSLQPEDFATYYCQ |
| | | QHYTTPPTFGQGTKVEIKRTVAAPSVFIFPPSDEQLKSGTASVVCLLNNFYPR |
| | | EAKVQWKVDNALQSGNSQESVTEQDSKDSTYSLSSTLTLSKADYEKHKVY |
| | | ACEVTHQGLSSPVTKSFNRGEC |
| Human | Heavy | LLQGLSGRSDNHGGGSQVQLVESGGGVVQPGRSLRLSCAASGFTFSSYTMH |
| CTLA-4 | (31) | WVRQAPGKGLEWVTFISYDGNNKYYADSVKGRFTISRDNSKNTLYLQMNS |
| | | LRAEDTAIYYCARTGWLGPFDYWGQGTLVTVSSASTKGPSVFPLAPSSKSTS |
| | | GGTAALGCLVKDYFPEPVTVSWNSGALTSGVHTFPAVLQSSGLYSLSSVVT |
| | | VPSSSLGTQTYICNVNHKPSNTKVDKRVEPKSC |
| | Light | GKGLSGRSDNHGGGSEIVLTQSPGTLSLSPGERATLSCRASQSVGSSYLAWY |
| | 32) | QQKPGQAPRLLIYGAFSRATGIPDRFSGSGSGTDFTLTISRLEPEDFAVYYCQ |
| | | QYGSSPWTFGQGTKVEIKRTVAAPSVFIFPPSDEQLKSGTASVVCLLNNFYP |
| | | REAKVQWKVDNALQSGNSQESVTEQDSKDSTYSLSSTLTLSKADYEKHKV |
| | | YACEVTHQGLSSPVTKSFNRGEC |
| Human | Heavy | LLQGLSGRSDNHGGGSQVQLVQSGVEVKKPGASVKVSCKASGYTFTNYYM |
| PD-1 | (33) | YWVRQAPGQGLEWMGGINPSNGGTNFNEKFKNRVTLTTDSSTTTAYMELK |
| | | SLQFDDTAVYYCARRDYRFDMGFDYWGQGTTVTVSSASTKGPSVFPLAPC |
| | | SRSTSESTAALGCLVKDYFPEPVTVSWNSGALTSGVHTFPAVLQSSGLYSLS |
| | | SVVTVPSSSLGTKTYTCNVDHKPSNTKVDKRV |
| | Light | GKGLSGRSDNHGGGSEIVLTQSPATLSLSPGERATLSCRASKGVSTSGYSYL |
| | (34) | HWYQQKPGQAPRLLIYLASYLESGVPARFSGSGSGTDFTLTISSLEPEDFAVY |
| | | YCQHSRDLPLTFGGGTKVEIKRTVAAPSVFIFPPSDEQLKSGTASVVCLLNNF |
| | | YPREAKVQWKVDNALQSGNSQESVTEQDSKDSTYSLSSTLTLSKADYEKH |
| | | KVYACEVTHQGLSSPVTKSFNRGEC |
| Mouse | Heavy | LLQGPLGLAGGSGGGSEAKLQESGPVLVKPGASVKMSCKASGYTFTDYYM |
| CTLA-4 | (224) | NWVKQSHGKSLEWIGVINPYNGDTSYNQKFKGKATLTVDKSSSTAYMELN |
| | | SLTSEDSAVYYCARYYGSWFAYWGQGTLITVSTAKTTPPSVYPLAPGCGDT |
| | | TGSSVTLGCLVKGYFPESVTVTWNSGSLSSSVHTFPALLQSGLYTMSSSVTV |
| | | PSSTWPSETVTCSVAHPASSTTVDKKLEP |
| | Light | GKGLSGRSDNHGGGSDIVMTQTTLSLPVSLGDQASISCRSSQSIVHSNGNTY |
| | (225) | LEWYLQKPGQSPKLLIYKVSNRFSGVPDRFSGSGSGTDFTLKISRVEAEDLG |
| | | VYYCFQGSHVPYTFGGGTKLEIKRADAAPTVSIFPPSSEQLTSGGASVVCFL |
| | | NNFYPKDINVKWKIDGSERQNGVLNSWTDQNSKDSTYSMSSTLTLTKDEYE |
| | | RHNSYTCEATHKTSTSPIVKSFNRNEC |

C. Exemplary Activatable Antibodies with Non-Covalently Associated Polypeptide Shield Moieties.

Exemplary activatable antibodies targeting human EGFR and human CTLA-4 are designed, in which each polypeptide shield moiety comprises protein fragments or complementary binding peptides. Non-covalent binding between the protein fragments or complementary binding peptides result in steric shields that block the antigen binding sites of the therapeutic antibodies. Sequences of the heavy chain and light chain of the exemplary activatable antibodies are shown in Table 12. A skilled person in the art would readily appreciate that other dimeric proteins, split-proteins, complementary binding peptides, and Fv fragments, disease-sensing releasable moieties (such as protease substrate peptides) and therapeutic antibodies described herein can be used to prepare similar activatable antibodies.

Exemplary Activatable Antibodies with a Dimeric Protein as Steric Shield

Exemplary activatable antibodies with a dimeric protein as the steric shield are prepared. The polypeptide shield moieties, which contain monomeric subunits of a protein dimer, are genetically fused to the N-termini of the heavy chain and light chain of a therapeutic antibody via a disease-sensing releasable moiety. The monomeric subunits associate to form a homo- or hetero-dimer, thereby joining the N-termini of the heavy chain and light chain of the therapeutic antibody. In one exemplary construct, an IL-10 monomeric subunit (e.g., SEQ ID NO 8) is fused to each of the N-termini of the heavy chain and light chain of an exemplary anti-EGFR antibody via a tumor-sensing releasable moiety (e.g., SEQ ID NO. 19). In another exemplary construct, a CXCL4 monomeric subunit (e.g., SEQ ID NO: 9) is fused to each of the N-termini of the heavy chain and light chain of an exemplary anti-EGFR antibody via a tumor-sensing releasable moiety (e.g., SEQ ID NO: 19). In a third exemplary construct, a CXCL5 monomeric subunit (e.g., SEQ ID NO: 19) is fused to each of the N-termini of the heavy chain and light chain of an exemplary anti-EGFR antibody via a tumor-sensing releasable moiety (e.g., SEQ ID NO: 19).

Monomeric subunits of heterodimers can also be used to generate activatable antibodies. For example, IL-12 is hetero-dimer where two monomeric subunits comprising amino acid sequences SEQ ID NO: II and SEQ ID NO: 12 respectively. In one exemplary construct, a polypeptide having the amino acid sequence of SEQ ID NO: 11 is genetically fused to the N-terminus of VH of an exemplary anti-EGFR antibody via a tumor-sensing releasable moiety (e.g., SEQ ID NO: 19), and a polypeptide having the amino acid sequence of SEQ ID NO: 12 is genetically fused to the N-terminus of VL of the exemplary anti-EGFR antibody via a tumor-sensing releasable moiety (e.g., SEQ ID NO: 19). Although a large number of protein dimers are suitable as steric shields, IL-10, IL-12, CXCL4 and CXCL5 have been chosen as examples as they could deliver therapeutic benefit on their own once released from the activatable antibodies in a diseased tissue.

Exemplary Activatable Antibodies with a Split-Protein as Steric Shield

Exemplary activatable antibodies with a split-protein as the steric shield are prepared. The split-protein is separated into two polypeptides that associate to form a whole split-protein fused to the N-termini of the heavy chain and light chain of an exemplary anti-EGFR antibody via releasable linker disease-sensor moiety. In an exemplary construct. Cub (e.g., SEQ ID NO: 6) is genetically fused to the N-terminus of the heavy chain of an exemplary anti-EGFR antibody via a tumor-sensing releasable moiety (e.g., SEQ ID NO: IQ), and Nub (e.g., SEQ ID NO: 7) is genetically fused to the N-terminus of the heavy chain of the exemplary anti-EGFR antibody via a tumor-sensing releasable moiety (e.g., SEQ ID NO: 19). Cub and Nub are complementary half fragments of split ubiquitin (Johnsson, N. & Varshavsky, A. 1994 Proc. Natl. Acad Sci. USA 91, 10340-10344). Upon expression, the two halves (Nub and Cub) associate to form ubiquitin, thereby joining the N-termini of the heavy and light chains of the exemplary anti-EGFR antibody and sterically blocking its antigen binding sites.

In another exemplary construct. SpyCatcher (e.g., SEQ ID NO: 4) is genetically fused to the N-terminus of the heavy chain of an exemplary anti-EGFR antibody via a tumor-sensing releasable moiety (e.g., SEQ ID NO: 19), and SpyTag (e.g., SEQ ID NO: 5) is genetically fused to the N-terminus of the heavy chain of the exemplary anti-EGFR antibody via a tumor-sensing releasable moiety (e.g., SEQ ID NO: 10). Upon expression, the two halves (SpyCatcher and SpyTag) associate to form ubiquitin, thereby joining the N-termini of the heavy and light chains of the exemplary anti-EGFR antibody and sterically blocking its antigen binding sites.

Exemplary Activatable Antibodies with a Coiled-Coil Complex as Steric Shield

Exemplary activatable antibodies with a coiled-coil complex as the steric shield are prepared. The heavy and light chains of the activatable antibodies carry peptides that bind to each other and form parallel or antiparallel coiled-coils. In an exemplary construct, Myo 10 anti-CC peptide (e.g., SEQ ID NO: 13), JIP3 fragment (e.g., SEQ ID NO: 14) or KIF21A (e.g., SEQ ID NO. 15) is genetically fused to each of the N-termini of the heavy chain and light chain of an exemplary anti-EGFR antibody via a tumor-sensing releasable moiety (e.g., SEQ ID NO. 19).

Heterodimer coiled-coils can also be used as steric shields. In an exemplary construct, MST1-RASSF5 SARAH peptide A (e.g., SEQ ID NO: 16) is genetically fused to the N-terminus of the heavy chain of an exemplary anti-EGFR antibody via a tumor-sensing releasable moiety (e.g., SEQ ID NO: 19), and MST1-RASSF5 SARAH peptide B (e.g., SEQ ID NO: 17) is genetically fused to the N-terminus of the light chain of the exemplary anti-EGFR antibody via a tumor-sensing releasable moiety (e.g., SEQ ID NO. 19).

Exemplary Activatable Antibodies with an Fv as Steric Shield

Exemplary activatable antibodies with an Fv fragment as the steric shield are prepared. For example, Fv (also known as “variable fragment”) of an exemplary anti-ED-B antibody comprises a VH having an amino acid sequence of SEQ ID NO: 35, and a VL having an amino acid sequence of SEQ ID NO: 36. The L19 Fv is a previously described human recombinant antibody specific for the ED-B domain of fibronectin isoform B-FN, which targets tumor blood vessels (Tarli L et al. 1999. Blood. 94: 192-8). In an exemplary construct, an activatable anti-CTLA4 antibody is designed. The VH1 of L19 (e.g., SEQ ID NO: 35) is genetically fused to the N-terminus of the heavy chain of an exemplary anti-CTLA-4 antibody via a tumor-sensing releasable moiety (e.g., SEQ ID NO: 19), and the VL of L19 (e.g., SEQ ID NO: 36) is genetically fused to the N-terminus of the light chain of the anti-CTLA-4 antibody via a tumor-sensing releasable moiety (e.g., SEQ ID NO:19). Fusion of the 119 Fv to the anti-CTLA-4 antibody prevents binding of the activatable anti-CTLA-4 antibody to its antigen CTLA-4 until L19 Fv is released. The N-termini of the heavy chain and light chain of the parent anti-CTLA-4 antibody have been engineered, with a few amino acid residues deleted from the native sequence to shorten the distance between the two N-termini. The shield moiety L19 Fv has two-fold capacities: 1) to block CTLA-4 binding by the activatable anti-CTLA-4 antibody outside tumor, and 2) to concentrate the activated anti-CTLA-4 antibody, in tumor by unblocking the CTLA-4 binding site in the activatable anti-CTLA-4 antibody in tumor tissue only.

TABLE 12
Exemplary activatable antibodies comprising split-proteins, protein dimers and
peptide coiled coil as steric shields.

| | | Chain | |
|---|---|---|---|
| | Steric | (SEQ | |
| target | shield | ID NO) | Sequence |
| Human | IL-10 | Heavy | SPGGGTQSENSCTHEPGNLPNMLRDLRDAESRVKTFFGMKDGLDNL |
| EGFR | dimer | (37) | LLKESLLEDFKGYLGCQALSEMIQFYLEEVMPQAENGDPDIKAHVN |
| | | | SLGENLKTLRLRLRRCHRFLPCENKSKAVEGVKNAFNKLGEKGIYK |
| | | | AMSEFDIFINYIEAYMTMKIRNLSGRSDNHQVGLKQSGPGLVQPSQS |
| | | | LSITCTVSGFSLTNYGVHWVRQSPGKGLEWLGVIWSGGNTDYNTPF |
| | | | TSRLSINKDNSKSQVFFKMNSLQSEDTAIYYCARALTYYDYEFAYW |
| | | | GQGTLVTVSAASTKGPSVFPLAPSSKSTSGGTAALGCLVKDYFPEPV |
| | | | TVSWNSGALTSGVHTFPAVLQSSGLYSLSSVVTVPSSSLGTQTYICN |
| | | | VNHKPSNTKVDKKVEPKSCDKTHTCPPCPAPELLGGPSVFLFPPKPK |
| | | | DTLMISRTPEVTCVVVDVSHEDPEVKENWYVDGVEVHNAKTKPRE |
| | | | EQYNSTYRVVSVLTVLHQDWLNGKEYKCKVSNKALPAPIEKTISKA |
| | | | KGQPREPQVYTLPPSRDELTKNQVSLTCLVKGFYPSDIAVEWESNG |
| | | | QPENNYKTTPPVLDSDGSFFLYSKLTVDKSRWQQGNVFSCSVMHE |
| | | | ALHNHYTGKSLSLSPG |
| | | Light | SPGQGTGSENSCTHEPGNLPNMLRDLRDAFSRVKTFFGMKDGLDNL |
| | | (38) | LLKESLLEDEKGYLGCGALSEMIGFYLEEVMPQAENGDPDIKAHVN |
| | | | SLGENLKTLRLRLRRCHRFLPCENKSKAVEQVKNAFNKLGEKGIYK |
| | | | AMSEFDIFINYIEAYMTMKIRNLSGRSDNHDILLTGSPVILSVSPGER |
| | | | VSFSCRASQSIGTNIHWYQQRINGSPRLLIKYASESISGIPSRFSGSGS |
| | | | GTDFTLSINSVESEDIADYYCQQNNNWPTTFGAGTKLELKRTVAAP |
| | | | SVFIFPPSDEQLKSGTASVVCLLNNFYPREAKVGWKVDNALQSGNS |
| | | | QESVTEQDSKDSTYSLSSTLTLSKADYEKHKVYACEVTHQGLSSPV |
| | | | TKSFNRGEC |
| | Split | Heavy | MQIFVKTLTGKTITLEVEPSDTIENVKAKIGDKELSGRSDNHQVGLK |
| | ubiquitin | (39) | QSGPGLVQPSQSLSITCTVSGFSLTNYGVHWVRQSPGKGLEWLGVI |
| | (Nub/Cub) | | WSGGNTDYNTPFTSRLSINKDNSKSQVFFKMNSLQSEDTAIYYCAR |
| | | | ALTYYDYEFAYWGQGTLVTVSAASTKGPSVFPLAPSSKSTSGGTAA |
| | | | LGCLVKDYFPEPVTVSWNSGALTSGVHTFPAVLQSSGLYSLSSVVT |
| | | | VPSSSLGTQTYICNVNHKPSNTKVDKKVEPKSCDKTHTCPPCPAPEL |
| | | | LGGPSVFLFPPKPKDTLMISRTPEVTCVVVDVSHEDPEVKFNWYVD |
| | | | GVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTGKSLSLSPG |
| | | Light | GIPPDGGRLIFAGKGLEDGRTLSDYNIGKESTLHLVLRLRGGLSGRS |
| | | (40) | DNHDILLTGSPVILSVSPGERVSFSCRASQSIGTNIHWYGGRINGSPR |
| | | | LLIKYASESISGIPSRFSGSGSGTDFTLSINSVESEDIADYYCGGNNNW |
| | | | PTTFGAGTKLELKRTVAAPSVFIFPPSDEQLKSGTASVVCLLNNFYP |
| | | | REAKVGWKVDNALQSGNSQESVTEGDSKDSTYSLSSTLTLSKADYE |
| | | | KHKVYACEVTHQGLSSPVTKSFNRGEC |
| | CXCL4 | Heavy | EAEEDGDLGCLCVKTTSGVRPRHITSLEVIKAGPHCPTAQLIATLKN |
| | dimer | (41) | GRKICLDLGAPLYKKIIKKLLESLSGRSDNHQVQLKQSGPGLVQPSQ |
| | | | SLSITCTVSGFSLTNYGVHWVRQSPGKGLEWLGVIWSGGNTDYNTP |
| | | | FTSRLSINKDNSKSQVFFKMNSLQSEDTAIYYCARALTYYDYEFAY |
| | | | WGQGTLVTVSAASTKGPSVFPLAPSSKSTSGGTAALGCLVKDYFPE |
| | | | PVTVSWNSGALTSGVHTFPAVLQSSGLYSLSSVVTVPSSSLGTQTYI |
| | | | CNVNHKPSNTKVDKKVEPKSCDKTHTCPPCPAPELLGGPSVFLFPPK |
| | | | PKDTLMISRTPEVTCVVVDVSHEDPEVKFNWYVDGVEVHNAKTKP |
| | | | REEQYNSTYRVVSVLTVLHQDWLNGKEYKCKVSNKALPAPIEKTIS |
| | | | KAKGQPREPQVYTLPPSRDELTKNQVSLTCLVKGFYPSDIAVEWES |
| | | | NGQPENNYKTTPPVLDSDGSFFLYSKLTVDKSRWQQGNVFSCSVM |
| | | | HEALHNHYTGKSLSLSPG |
| | | Light | EAEEDGDLGCLCVKTTSGVRPRHITSLEVIKAGPHCPTAQLIATLKN |
| | | (42) | GRKICLDLQAPLYKKIIKKLLESLSGRSDNHDILLTQSPVILSVSPGER |
| | | | VSFSCRASQSIGTNIHWYGGRTNGSPRLLIKYASESISGIPSRESGSGS |
| | | | GTDFTLSINSVESEDIADYYCGGNNNWPTTFGAGTKLELKRTVAAP |
| | | | SVFIFPPSDEQLKSGTASVVGLLNNFYPREAKVGWKVDNALQSGNS |
| | | | QESVTEQDSKDSTYSLSSTLTLSKADYEKHKVYACEVTHQGLSSPV |
| | | | TKSFNRGEC |
| | Myo10 | Heavy | ENKGVEEILRLEKEIEDLGRMKEQQELSLTLSGRSDNHQVQLKQSGP |
| | anti-parallel | (43) | GLVQPSQSLSITCTVSGFSLTNYGVHWVRQSPGKGLEWLGVIWSGG |
| | coiled coil | | NTDYNTPFTSRLSINKDNSKSQVFFKMNSLQSEDTAIYYCARALTYY |
| | | | DYEFAYWGQGTLVTVSAASTKGPSVFPLAPSSKSTSGGTAALGCLV |
| | | | KDYFPEPVTVSWNSGALTSGVHTFPAVLQSSGLYSLSSVVTVPSSSL |
| | | | GTQTYICNVNHKPSNTKVDKKVEPKSCDKTHTCPPCPAPELLGGPS |
| | | | VFLFPPKPKDTLMISRTPEVTCVVVDVSHEDPEVKFNWYVDGVEVH |
| | | | NAKTKPREEQYNSTYRVVSVLTVLHQDWLNGKEYKCKVSNKALP |
| | | | APIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNV |
| | | | FSCSVMHEALHNHYTQKSLSLSPG |
| | | Light | ENKQVEEILRLEKEIEDLGRMKEQQELSLTLSGRSDNHDILLTGSPVI |
| | | (44) | LSVSPGERVSFSCRASQSIGTNIHWYQQRINGSPRLLIKYASESISGIP |
| | | | SRFSGSGSGTDFTLSINSVESEDIADYYCGGNNNWPTTFGAGTKLEL |
| | | | KRTVAAPSVFIFPPSDEQLKSGTASVVCLLNNFYPREAKVQWKVDN |
| | | | ALQSGNSQESVTEQDSKGSTYSLSSTLTISKADYEKHKVYACEVTH |
| | | | GGLSSPVTKSFNRGEC |
| Human | L19 | Heavy | EVQLLESGGGLVQPGGSLRLSCAASGFTFSSESMSWVRQAPGKGLE |
| CTLA-4 | Fv | (45) | WVSSISGSSGTTYYADSVKGRFTISRDNSKNTLYLGMNSLRAEDTA |
| | | | VYYCAKPFPYFDYWGQGTLVTVLSGRSDNHVQLVESGGGVVQPGR |
| | | | SLRLSCAASGFTFSSYGMHWVRQAPGKGLEWVAVIWYDGSNKYY |
| | | | ADSVKGRFTISRDNSKNTLYLQMNSLRAEDTAVYYCARDPRGATL |
| | | | YYYYYGMDVWGQGTTVTVSSASTKGPSVFPLAPCSRSTSESTAALG |
| | | | CLVKDYFPEPVTVSWNSGALTSGVHTFPAVLQSSGLYSLSSVVTVPS |
| | | | SNFGTQTYTCNVDHKPSNTKVDKTVERKCCVECPPCPAPPVAGPSV |
| | | | FLFPPKPKDTLMISRTPEVTCVVVDVSHEDPEVQFNWYVDGVEVHN |
| | | | AKTKPREEQFNSTFRVVSVLTVVHQDWLNGKEYKCKVSNKGLPAPI |
| | | | EKTISKTKGQPREPQVYTLPPSREEMTKNQVSLTCLVKGFYPSDIAV |
| | | | EWESNGQPENNYKTTPPMLDSDGSFFLYSKLTVDKSRWQQGNVFS |
| | | | CSVMHEALHNHYTQKSLSLSPGK |
| | | Light | EIVLTQSPGTLSLSPGERATLSCRASQSVSSSYLAWYQQKPGQAPRL |
| | | (46) | LIYYASSRATGIPDRFSGSGSGTDFTLTISRLEPEDFAVYYCQQTGRIP |
| | | | PTFGGGTKVEILSGRSDNHIQMTQSPSSLSASVGDRVTITCRASQSIN |
| | | | SYLDWYQQKPGKAPKLLIYAASSLQSGVPSRFSGSGSGTDFTLTISSL |
| | | | QPEDFATYYCQQYYSTPFTFGPGTKVEIKRTVAAPSVFIFPPSDEQLK |
| | | | SGTASVVCLLNNFYPREAKVQWKVDNALQSGNSQESVTEQDSKDS |
| | | | TYSLSSTLTLSKADYEKHKVYACEVTHQGLSSPVTKSFNRGEC |

D. Exemplary Activatable Anti-EGFR Ab Constructs with a pH-Sensing or Protease-Sensing Steric Shield Comprising a Glycan-Binding Protein-Glycan Complex.

Some proteins undergo conformational changes as pH shifts from neutral towards acidic. For example, histidines within proteins can act as pH sensor and thus mediate pH-controlled structural changes. Tumor tissues are known to have lower extracellular pH than normal tissues, thus shield moiety wherein peptide shield moieties S1 and S2 are associated at normal tissue pH (e.g. ˜pH 7.4), and dissociated at tumor tissue pH (e.g. ˜pH 6.5 or lower) is designed.

M-ficolin is a human lectin that binds to N-acetylglucosamine (GlcNAc), where binding of M-ficolin to N-acetylglucosamine (GlcNAc) is subject to a pH-sensitive conformational switch. M-ficolin binds GlcNAc at normal physiological pH, but affinity starts to decrease at ˜pH 6.5 (Garlatti V. et al. 2008. J. Biol. Chem. 282: 35814-35820, Tanio M et al. J. Synchrotron Rad. 2008, 15, 243-245 Gout E. et al 2010. J. Biol Chem. 285: 6612-6622). Therefore, pH activatable anti-EGFR antibodies are designed. An engineered M-ficolin monomer (e.g., SEQ ID NO. 20) is directly fused to the N-terminus of the heavy chain of an exemplary anti-EGFR antibody, and an N-glycosylation sequence tag (e.g., SEQ ID NO: 18) is directly fused to the N-terminus of the light chain of the anti-EGFR antibody, as shown in Table 13. The N-glycosylation tag is glycosylated during expression of the fusion antibody in mammalian cell lines, and the glycans added to the glycosylation tag binds to M-ficolin at normal tissue pH thus providing a steric shield in the activatable anti-EGFR antibody. At lower than normal pH (e.g., in a tumor tissue), M-ficolin changes its conformation and releases the glycan, thereby releasing the steric shield and allowing the antigen binding site to bind its antigen. EGFR. In another exemplary construct, the engineered M-ficolin monomer (e.g., SEQ ID NO: 20) is genetically fused to the N-terminus of the heavy chain of the anti-EGFR antibody via a tumor-specific protease substrate (e.g., SEQ ID NO: 19), and the N-glycosylation sequence tag (e.g., SEQ ID NO: 18) is genetically fused to the N-terminus of the heavy chain of anti-the EGFR antibody via a tumor-specific protease substrate (e.g., SEQ ID NO: 19). Cleavage of the tumor-specific protease substrate (i.e., disease-sensing releasable moieties) release the M-ficolin-glycan complex, thereby unblocking the antigen binding site for EGFR.

TABLE 13
Exemplary activatable anti-EGFR antibody constructs comprising a glycan binding
protein-glycan sieric shield and a pH-sensing releasable moiety or protease-cleavable
disease-sensing releasable moiety.

| | Chain | |
|---|---|---|
| | (SEQ ID | |
| DS | NO) | Sequence |
| pH-sensing | Heavy | QSCATGPRNCKDLLDRGVERSGWHTIYLPDCRPLTVLCDMIDTDGGGWTV |
| releasable | (47) | FQRRMDGSVDFYRDWAAYKQGFGSQLGEFWLGNDNIHALTAQGSSELRT |
| moiety | | DLVDFEGNHQFAKYKSFKVADEAEKYKLVLGAFVGGSAGNSLTGHNNNF |
| | | FSTKDQDNDVSSSNCAEKFQGAWWYADCHASNLNGLYLMGPHESYANGI |
| | | NWSAAKGYKYSYKVSEMKVRPAQVQLKQSGPGLVQPSQSLSITCTVSGFSLT |
| | | NYGVHWVRQSPGKGLEWLGVIWSGGNTDYNTPFTSRLSINKDNSKSQVFFKM |
| | | NSLQSEDTAIYYCARALTYYDYEFAYWGQGTLVTVSAASTKGPSVFPLAPSSK |
| | | STSGGTAALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAKTKPRE |
| | | EQYNSTYRVVSVLTVLHQDWLNGKEYKCKVSNKALPAPIEKTISKAKGQPREP |
| | | QVYTLPPSRDELTKNQVSLTCLVKGFYPSDIAVEWESNGQPENNYKTTPPVLD |
| | | SDGSFFLYSKLTVDKSRWQQGNVFSCSVMHEALHNHYTQKSLSLSPG |
| | Light | SNYSMVNTTNMTSDILLTQSPVILSVSPGERVSFSCRASQSIGTNIHWYQQRTN |
| | (48) | GSPRLLIKYASESISGIPSRFSGSGSGTDFTLSINSVESEDIADYYCQQNNNWPTT |
| | | FGAGTKLELKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQGLSS |
| | | PVTKSFNRGEC |
| Disease-specific | Heavy | QSCATGPRNCKDLLDRGYFRSGWHTIYLPDCRPLTVLCDMDTDGGGWTV |
| protease | (49) | FQRRMDGSVDFYRDWAAYKQGFGSQLGEFWLGNDNIHALTAQGSSELRT |
| substrate | | DLVDFEGNHQFAKYKSFKVADEAEKYKLVLGAFVGGSAGNSLIGHNNNE |
| | | ESTKDQDNDVSSSNCAEKFQGAWWYADCHASNLNGLYLMGPHESYANGI |
| | | NWSAAKGYKYSYKVSEMKVRPALSGRSDNHQVQLKQSGPGLVQPSQSLSIT |
| | | CTVSGFSLTNYGVHWVRQSPGKGLEWLGVIWSGGNTDYNTPFTSRLSINKDNS |
| | | KSQVFFKMNSLQSEDTAIYYCARALTYYDYEFAYWGQGTLVTVSAASTKGPS |
| | | VFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPIEKTIS |
| | | KAKGQPREPQVYTLPPSRDELTKNQVSLTCLVKGFYPSDIAVEWESNGQPENN |
| | | YKTTPPVLDSDGSFFLYSKLTVDKSRWQQGNVFSCSVMHEALHNHYTQKSLS |
| | | LSPG |
| | Light | SNYSMVNTTNMTSLSGRSDNHDILLTQSPVILSVSPGERVSFSCRASQSIGTNIH |
| | (50) | WYQQRTNGSPRLLIKYASESISGIPSRESGSGSGTDFTLSINSVESEDIADYYCQQ |
| | | NNNWPTTFGAGTKLELKRTVAAPSVFIFPPSDEQLKSGTASVVCLLNNFYPREA |
| | | KVQWKVDNALQSGNSQESVTEQDSKDSTYSLSSTLTLSKADYEKHKVYACEV |
| | | THQGLSSPVTKSFNRGEC |

### Example 3. Structural Models of Polypeptide Steric Shields on Four Different Antibodies

Structural examination of antibodies showed that the N-termini of the heavy chain and light chain are positioned such that if connected by a moiety of sufficient length, that moiety can hoover over the antigen binding site and consequently prevent antigen binding. Structural modeling was performed to build a model of peptide-based steric shields generated when two polypeptides are genetically fused to the N-termini of the heavy chain and light chain, and then conjugated to each other.

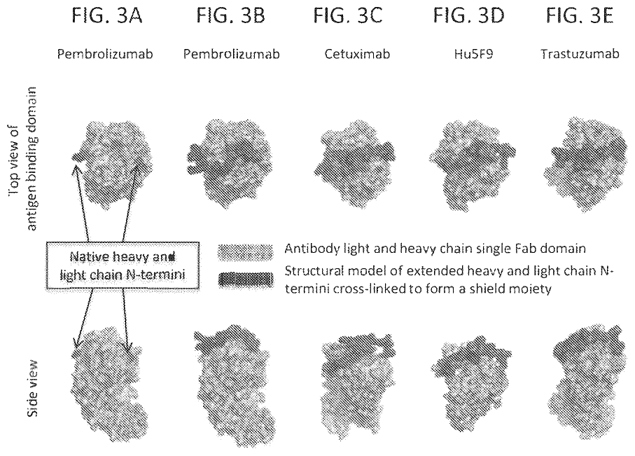

FIG. 3 shows structural models of peptide steric shields on four different antibodies that bind different antigens, including Pembrolizumab:PD1, Cetuximab:EGFR, Trastuzumab:HER-2, and Hu5F9:CD47. Shown are X-ray crystal structures of single Fab domains of the four different antibodies (light gray color) with structural models of peptide steric shields formed by extending the native N-termini with polypeptide shield moieties S1 and S2, and then conjugating S1 and S2 (dark grey color) by transglutamination or Cys-Cys disulfide bridge formation (i.e., reactions illustrated in FIG. 2A). Top view and side views of single Fab domains are shown. Top view is top-down view of the antigen binding site, and side view is 90° rotation of the top view. FIG. 3A shows the crystal structure of a single Fab domain of Pembrolizumab (PDBID: 5JXE) with arrows pointing to the native N-termini of the heavy chain and light chain. FIG. 3B shows the crystal structure of Pembrolizumab with structural models of the polypeptide shield moieties S1 and S2 wherein S1 and S2 have identical sequence, SCLSGRSDNHGG (SEQ ID NO: 145), added to each of the N-termini of the heavy chain and light chain A Cys-Cys disulfide bond was modeled to associate S1 and S2 in silico. In SEQ ID NO: 145, the association moeity B and C are SC (SEQ ID NO: 3), in which the cysteines form a Cys-Cys disulfide bridge and thus join the N-termini of the heavy chain and light chain. Both S1 and S2 carry the same disease-sensing release moiety (DS1 and DS2) LSGRSDNH (SEQ ID NO: 19), which is a substrate of matriptase and uPA.

FIGS. 3C-3E show crystal structures of Cetuximab (PDBID: 1YY9) (FIG. 3C), Hu5F9 (PDBID: 51WL) (FIG. 3D) and Trastuzumab (PDBID 1N8Z) (FIG. 3E) with structure models of identical polypeptide shield moieties S1 and S2 added to the N-termini of the heavy chain and light chain of each antibody respectively. Structural model of polypeptide shield moiety (S1), LLQGLSGRSDNH (SEQ ID NO. 140), was added to the native N-terminus of the heavy chain Cetuximab, Trastuzumab and Hu5F9 respectively, and structural models of polypeptide shield moiety (S2) GKGLSGRSDNH (SEQ ID NO: 141), was added to the native N-terminus of the light chain of Cetuximab. Trastuzumab and Hu5F9, respectively. Polypeptide shield moiety S1 carries the association moiety B having the sequence LLQG (SEQ ID NO: 1), an acyl donor tag; and polypeptide shield moiety S2 carries C having the sequence GKG (SEQ ID NO: 2), an acyl acceptor tag. Both S1 and S2 carries the disease-sensing release moiety (DS1 and DS2) LSGRSDNH (SEQ ID NO: 19), which is a substrate of matriptase and uPA. An isopeptide bond residues Q in S1 and K in S2 was modeled to associate S1 with S2 in silico.

Results of the structural modeling of the steric shields on four antibodies that bind different targets clearly demonstrate that connecting the N-termini of the heavy chain and light chain via peptide moieties of sufficient length generated steric shields that sit across the antigen binding sites for all four antibodies. Thus, presence of the peptide shields will prevent antigen binding of the antibodies.

### Example 4. Steric Shield Generation by Transglutaminase-Catalyzed Conjugation of Polypeptide Shield Moieties

Steric shields were formed on two exemplary activatable anti-EGFR antibodies (referred herein as “activatable EGFR Ab”), constructs 1 and 2 of Table 10, by treating the corresponding fusion antibodies with transglutaminase under different reaction conditions.

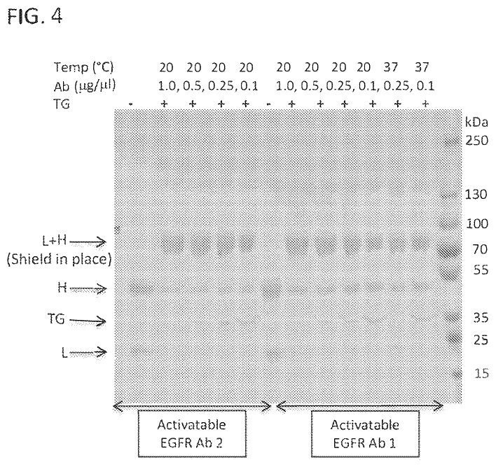

The following reaction parameters were used: transglutaminase concentration range (0.25, 0.5, 0.75, 1.0, 2.0% wt/v), fusion antibody concentration range (0, 1. 0.25, 0.5 and 1.0 mg/ml), NaCl concentration (20, 150 and 300 mM). pH (7.4, 8.0, 8.5) and temperature of reaction (20, 37° C.) for 12 hr. Efficiency of transglutaminase-catalyzed steric shield formation was analyzed using 4-12% Tris-Glycine SDS-PAGE gels. FIG. 4 shows results fora subset of tested conditions. The 4-12% SOS-PAGE gel was ran in the presence of reducing agent (TCEP) so that heavy chain (marked by letter H) and light chain (marked by letter L) in conventional antibodies ran as separate bands, around ˜50 and ˜25 kDa respectively. However, activatable antibodies having conjugated steric shield yielded a band corresponding to conjugated heavy chain and light chain species at about ˜75 kDa. On top of the gel image, absence or presence of transglutaminase (TG) in the reaction is marked by minus (−) or plus (+) sign, and fusion antibody concentrations (Ab μg/μl) and reaction temperature for each reaction are indicated.

Notably, in the presence of transglutaminase, the heavy chain and light chain in the activatable antibody were conjugated, and thus ran as the 75 kDa specie (marked as H+L). Thus, this data clearly demonstrates that the polypeptide shield moieties fused to the N-termini of the heavy chain and light chain of the anti-EGFR antibody were conjugated by the transglutaminase, thereby providing a complete steric shield.

### Example 5. In Vitro Removal or Transglutaminase-Conjugated Steric Shield by Protease Cleavage of Tumor-Sensing Releasable Moieties

In order to determine whether the activatable EGFR Ab 1 and 2 could be activated specifically in tumor tissues, in vitro experiments were performed using proteases uPA and matriptase, which are commonly known to be up-regulated in cancers.

In order to optimize cleavage conditions, activatable EGFR Ab 1 and 2, were each incubated with matriptase or uPA at a protease:antibody concentration ratio of 1:10, 1:100, or 1:1000 (mole:mole) in 50 mM Tris pH 8.5, 0.01% Tween-20 buffer for 18 hrs at 37° C. or at a protease:antibody concentration ratio of 1:100 in 100 mM Tris-HCl pH 8.0, 150 mM NaCl or 100 mM Tris-HCl pH 8.5, 150 mM NaCl buffers. The cleavage reaction was stopped by adding SOS-PAGE sample buffer and heating sample at 95° C. for 5 minutes. Effects of matriptase or uPA-specific digestion were evaluated by running samples on reducing 4-12% Tris-Glycine SDS-PAGE gels (Thermo Fisher) in the presence of the reducing agent TCEP, so that heavy (marked by letter H) and light (marked by letter L) chains ran as separate bands, ˜50 and ˜25 kDa respectively, unless conjugated (marked as H+L, 75 kDa specie).

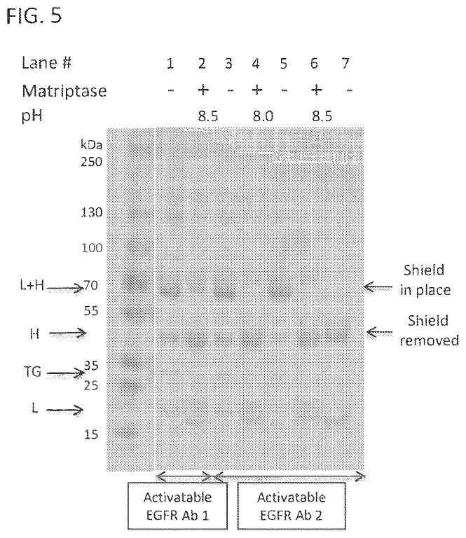

Results are shown in FIG. 5. For samples in lanes 1-6, steric shields were first formed by incubation with a transglutaminase (TG) in separate reactions, followed by removal of the steric shields by cleavage of the tumor-sensing releasable moiety (SEQ ID NO: 19) by matriptase at pH 8.5 or pH 8.0. The minus (−) or plus (+) signs on top of the gel image indicate absence or presence of matriptase in each reaction. Lane 7 shows a control sample in which the antibody was not subjected to transglutaminase treatment, and thus a steric shield was never formed prior to matriptase treatment. As a result, in lane 7, the heavy (H) and light (L) chains ran as separate bands. Notably, in the presence of matriptase, the fraction of conjugated heavy and light chains (H+L; ˜75 kDa band) decreased, while the fraction of heavy (H) and light (L) chains that ran as separate bands (˜50 kDa and ˜25 kDa respectively) increased as the relative concentration of matriptase in the reaction increased. Thus, this data demonstrates that the transglutaminase-conjugated steric shield can be removed via cleavage of the disease-sensing releasable moieties by a disease-specific protease.

### Example 6. Steric Shield Generation by Cys-Cys Disulfide Bridge Formation Between Polypeptide Shield Moieties During Expression in Mammalian Cells

Eukaryotic protein expression systems support disulfide bond formation in eukaryotic proteins. In particular, mammalian expression systems using HEK293 and CHO cell lines have been extensively used in the art for expression of antibodies with their native disulfide bonds in place. Two exemplary activatable EGFR Abs (constructs 3 and 4 of Table 10) have been designed. The cysteines in B and C were designed to be in the vicinity of each another so that they could form a disulfide bridge during expression in mammalian cells.

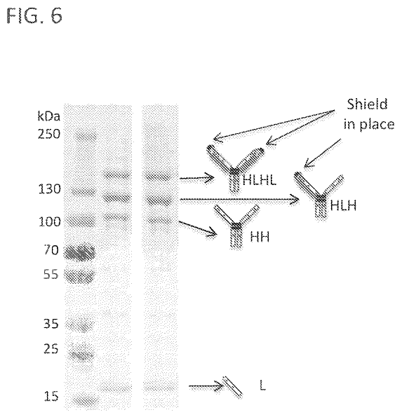

The fusion antibody constructs, were expressed in Expi293F cells. After protein purification, non-reducing SDS-PAGE gels were used to analyze disulfide bridge mediated steric shield formation during antibody expression in Expi293F cells. FIG. 6 shows 4-12% Tris-Glycine SDS-PAGE gel (ThermoFisher) image ran in the absence of reducing agent, thus Cys-Cys disulfide bridges (shown as black lines in the antibody cartoons) remained intact. Antibody constructs with heavy and light chains SEQ ID NO: 25 and 26, and SEQ ID NO: 27 and 28 are designed to carry Cys/Ser mutation in their heavy (H) and light (L) chains such that native heavy-light (HL) Cys-Cys disulfide bridge could not form, while native heavy-heavy chain (HH) disulfide bridges still formed. Thus, it was expected to observe the following species on non-reducing SDS-PAGE gel: ˜25 kDa light chain species (in FIG. 6 labeled L and shown as cartoon), ˜100 kDa heavy-heavy chain species (in FIG. 6 labeled HH and shown as cartoon) if the steric shield did not form, ˜125 kDa species if the Cys-Cys steric shield formed on one of the two antigen-binding-sites (in FIG. 6, labeled HHL and shown as cartoon) and ˜150 kDa species if the Cys-Cys steric shield formed on both antigen binding sites (in FIG. 6, labeled HLHL and shown as cartoon).

SDS-PAGE gel showed the presence of four species: ˜150 kDa HLHL species (2 steric shields formed); ˜125 kDa HLH species (1 steric shield formed); ˜100 kDa HH species (no steric shield formed); and ˜25 kDa light chain species. Data shown in FIG. 6 demonstrated that Cys-Cys conjugated steric shields formed on both antigen-binding sites or on one of the two antigen binding sites during expression in mammalian cells.

### Example 7. Steric Shield Generation by Cys-Cys Disulfide Bridge Formation Between Polypeptide Shield Moieties by Reduction/Oxidation

During protein expression, cysteines introduced to the N-termini of antibodies can complex with free cysteine or glutathione amino acids present in the expression media (Junutula J R et al. 2008. Nature Biotech. 26, 925-932), thus blocking said cysteines from forming a disulfide bond with the desired partner(s). Such cysteines can be unconjugated from cysteine or glutathione amino acids by reduction using TCEP followed by oxidation using ascorbic acid (Junutula J R et al. 2008. Nature Biotech. 26, 925-932).

Here, the fusion antibodies (activatable EGFR Ab 3 and 4) expressed in mammalian cells was incubated with TCEP (ThermoFisher) at a range of concentration ratios between the fusion antibody and TCEP including 1:1, 1:3, 1:5, 1.10 or 1:20 (mole:mole) for 30 or 60 min at 20° C. or 4° C. TCEP was removed by dialysis for 3 hr at mom temperature in 1×PBS buffer, using SLIDE-A-LYZER™ MINI Dialysis Device (ThermoFisher). The N-terminal Cys-Cys disulfide bond between the polypeptide shield moieties was formed by addition of dihydroascorbic acid (dhAA) (Sigma-Aldrich) to the dialyzed antibodies at TCEP:dhAA ratios (mole:mole) of 1:3 or 1:5 at 20° C. or 4° C. In addition to TCEP:dhAA, other reduction/oxidation reagents can be used, e.g., reduced/oxidized glutathione.

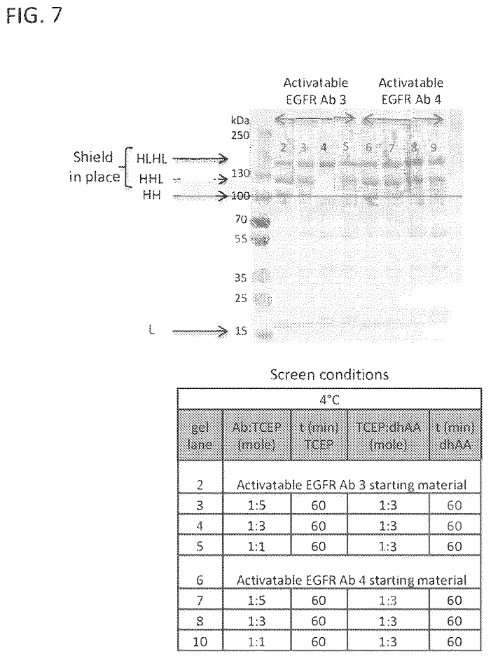

Formation of the Cys-Cys disulfide bond in the steric shield after TCEP/dHAA incubation was assessed using non-reducing 4-12% SDS-PAGE gels. Results from a subset of reaction conditions are shown in FIG. 7. As discussed in Example 6, four distinct species may be observed on the SDS-PAGE gel: ˜25 kDa light chain (in FIG. 7 labeled L); ˜100 kDa heavy-heavy chain species (in FIG. 7 labeled HH), when the steric shield did not form; ˜125 kDa species if the steric shield formed on one of the two antigen-binding sites (in FIG. 7, labeled HHL), and ˜150 kDa species if the steric shield formed on both antigen binding sites (in FIG. 7, labeled HLHL).

Samples in lanes 3-5 and 7-9 were first reduced by incubation with TCEP under conditions listed in the accompanying table in FIG. 7, then TCEP was dialyzed and ascorbic acid (dhAA) added under conditions indicated in the accompanying table in FIG. 7. Comparison of lanes 2 (starting material) and lanes 3-5 for activatable EGFR Ab construct 3 and lanes 6 (starting material) and lanes 7-10 for EGFR Ab construct 4 showed that reduction followed by oxidation with TCEP-dhAA changed the ratio among 3 species: HH (no steric shield), HHL (one steric shield) and HLHL (two steric shields). Fractions of the HH species (no steric shield formed) decreased, while fractions of the HLHL (two steric shields formed) were enriched as the Antibody:TCEP ratio in the reduction step decreased. This data demonstrates that the Cvs-Cvs disulfide-conjugated steric shield can be re-formed in vitro after expression.

### Example 8. In Vitro Removal of Cys-Cys Disulfide-Conjugated Steric Shield by Cleavage of Tumor-Sensing Releasable Moieties

In order to determine whether the activatable EGFR Ab 3 and 4 could be activated specifically in tumor tissues, in vitro experiments were performed using proteases uPA and matriptase, which are commonly known to be up-regulated in cancers.

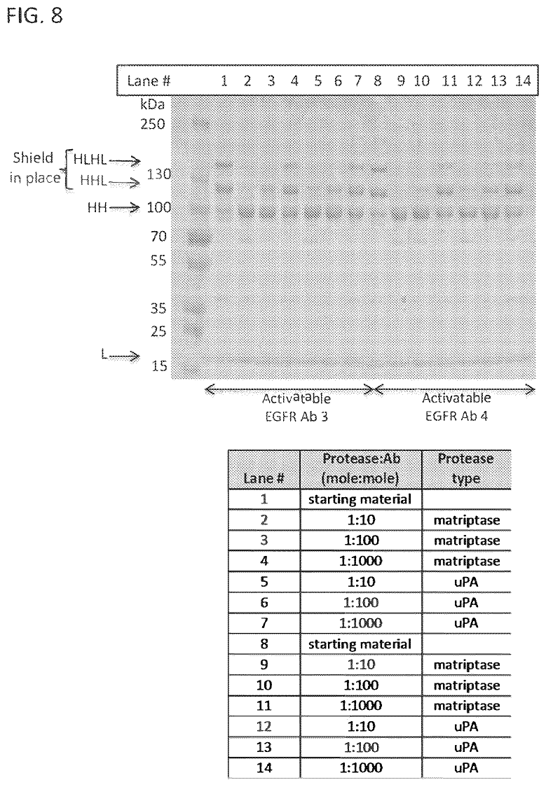

In order to optimize cleavage conditions, activatable EGFR Ab 3 and 4 were incubated with matriptase or uPA at a range of protease, antibody ratios (1:10, 1:100, 1:1000 (mole:mole)) in 50 mM Tris pH 8.5, 0.01% Tween-20 buffer for 18 hrs at 37° C. The cleavage reaction by matriptase or uPA was stopped by adding SDS-PAGE sample buffer, and the samples were subsequently heated at 95° C. for 5 minutes. Effects of matriptase or uPA specific digestion was evaluated by running the samples on non-reducing 4-12% Tris-Glycine SDS-PAGE gels (Thermo Fisher) so that any disulfide-bridges would remain intact. Results are shown in FIG. 8.

Non-reducing SDS-PAGE gel analysis of the starting material (FIG. 6 and FIG. 8 lanes 1 and 8) before treatment with matriptase or uPA showed the ˜150 kDa HLHL species and 125 kDa HHL species, which correspond to activatable antibodies having both or one antigen binding site covered by the Cys-Cys disulfide-conjugated steric shield, respectively. After incubation with matriptase (lanes 2-4 and lanes 9-12) or uPA (lanes 5-7 and lanes 12-14), the ˜125 kDa HHL species (one steric shield) and the ˜150 kDa HLHL species (two steric shields) decreased with increasing protease concentration. This data clearly demonstrates that the activatable EGFR Ab 3 and 4 can be cleaved by matriptase or uPA to release the Cys-Cys conjugated steric shield in vitro. By comparing the amount of the uncleaved activatable antibody (HHL and HLHL species) to that of completely cleaved species (HH), it appears that matriptase was more efficient than uPA to cleave the activatable antibodies, i.e., resulting in more complete cleavage.

### Example 9. Antigen Binding to Activatable Antibodies in the Presence and Absence of Steric Shields

Binding of human EGFR to activatable EGFR Ab 1, 2, 3, and 4 that either did or did not have steric shields in place (i.e., conjugation between S1 and S2 were formed or not formed), and protease-treated activatable antibodies were analyzed on a PROTEON™ XPR36 Surface Plasmon Resonance (SPR) Instrument (BioRad) using phosphate buffered saline (pH 7.4) supplemented with 0.01% Tween-20 as running buffer.

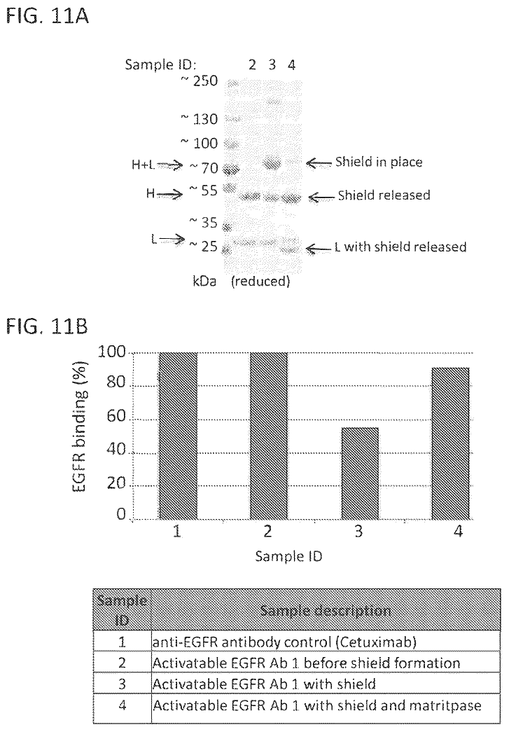

For transglutaminase-catalyzed shield formation, 1.0 and 0.5 mg/ml of fusion antibody comprising SEQ ID NOs: 21 and 22 (activatable EGFR Ab 1) or fusion antibody comprising SEQ ID NOs: 23 and 24 (activatable EGFR Ab 2) was incubated with 0.75% wt/v transglutaminase enzyme in 100 mM Tris-HCl pH 8.5, 150 mM NaCl buffer for 12 hrs at 18° C. Afterwards, transglutaminase was removed from activatable EGFR Ab 1 or EGFR Ab 2 by protein A affinity chromatography. Representative results of shield formation of activatable EGFR Ab 1 is show-n on a reduced SDS-PAGE gel in FIG. 11A (lane labeled sample ID 3) Reducing SDS-PAGE gel analysis of the starting (lane labeled sample ID 2) and transglutaminase cross-linked material (lane labeled sample ID 3) clearly demonstrates formation of ˜70 kDa HL species with conjugated heavy and light chains as discussed in Example 4

For Cys-Cys disulfide bridge mediated shield formation, the Cys-Cys disulfide bridge on activatable EGFR Ab 3 and 4 was reformed post-expression as described in Example 7, and purified using chromatography. For the shown SPR data incubation conditions with TCEP/dhAA, Ab:TCEP ratios were 1:10 and 1:20 for 30 min at 20° C., and 1:5 for 60 mm at 4° C., followed by TCEP:dhAA of 1:3 for 60 or 180 min.

After steric shield formation, for n vitro shield removal (FIGS. 11A-11B), activatable EGFR Ab 1 with shield moiety in place was incubated with matriptase protease at protease:antibody ratio of 1:50 (mole:mole) in 50 mM Tris pH 8.5, 0.01% Tween-20 buffer for 18 hrs at 37° C. Representative result of shield removal of activatable EGFR Ab 1 is shown on a reduced SDS-PAGE gel in FIG. 11A (lane labeled sample ID 4). Reducing SDS-PAGE cl analysis of the starting and matriptase treated material (lane labeled sample ID 3 and lane labeled sample ID 4) clearly demonstrates that activatable EGFR Ab 1 cleaved by matriptase releases the transglutaminase conjugated steric shield in vitro, as discussed in Example 5.

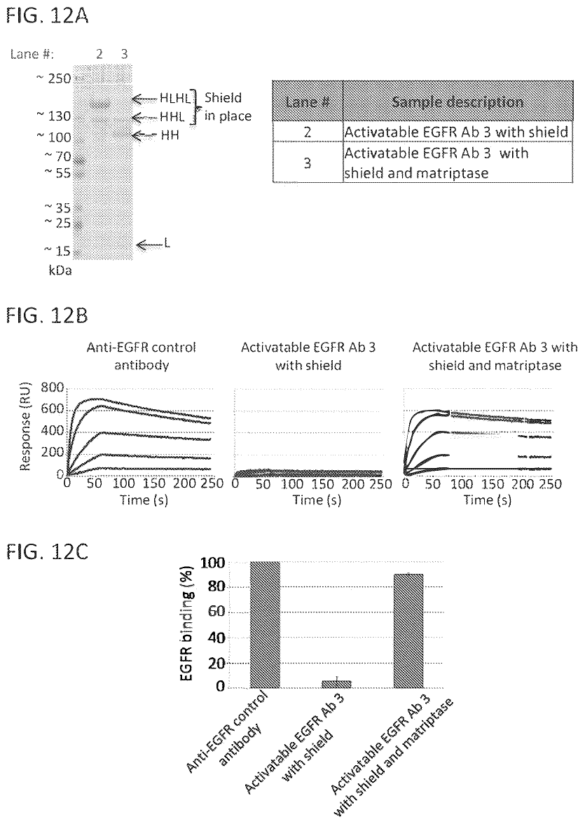

In another experiment (FIGS. 12A-12C), activatable EGFR Ab 3 with shield moiety in place was incubated with matriptase protease at protease:antibody ratio of 1:50 (mole:mole) in PBS pH 7.4 for 18 hr at 37° C. FIG. 12A shows a non-reducing 4-12% Tris-Glycine SDS-PAGE gel demonstrating Cys-Cys disulfide bridge formation (lane labeled as sample ID: 2), and results of subsequent shield removal mediated by matriptase cleavage of the tumor-sensing moieties (SEQ ID NO: 19). Non-reducing SDS-PAGE gel analysis of the starting material (FIG. 12A lane labeled SAMPLE ID 2 before treatment with matriptase) shows that the majority of sample were the ˜150 kDa HLHL species and ˜125 kDa HHL, species, which correspond to activatable antibodies having both or one antigen binding site covered by the Cys-Cys disulfide-conjugated steric shield, respectively. After incubation with matriptase (FIG. 12A lane labeled SAMPLE ID 3) the ˜25 kDa HHL species (one steric shield) and the ˜150 kDa HLHL species (two steric shields) decreased, while the ˜100 kDa HH species (no steric shield) increased. This data clearly demonstrates that the activatable EGFR Ab 3 cleaved by matriptase releases the Cys-Cys conjugated steric shield in vitro, as discussed in Example 8.

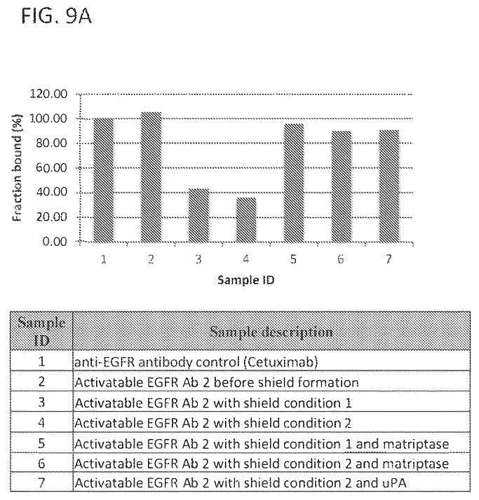

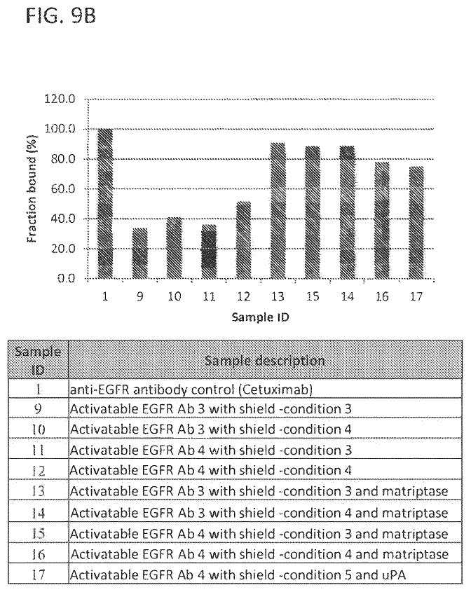

In the experiments shown in FIGS. 9A-9B, activatable EGFR Ab 2-4 with shield moiety in place were incubated with either matriptase or uPA protease at protease antibody ratio of 1:15 (mole:mole) in 50 mM Tris pH 8.5, 0.01% Tween-20 buffer for 18 hrs at 37° C.

Activatable EGFR Ab constructs and the native anti-EGFR antibody were attached to chips via Protein G, and binding to their antigen, EGFR, was measured. The amount of EGFR-binding by the activatable EGFR Abs was normalized to EGFR binding by the parent anti-EGFR antibody, and shown as bar graphs. EGFR-binding efficiency was evaluated before steric shield formation (activatable EGFR Ab 1-2), after shield formation, and after protease-specific shield removal (activatable EGFR Ab 1-4).

FIGS. 9A and 11B show bar graphs illustrating differences in binding activity of activatable EGFR Ab 2 and activatable EGFR Ab 1, respectively, to its target, EGFR, before and after shield moiety formation via transglutamination, and before and after in vitro release of the steric shield via cleavage of the tumor sensing releasable moiety by protease. The results demonstrate: 1) the presence of unconjugated polypeptide shield moieties at the N-termini of the anti-EGFR antibody did not hinder EGFR binding since the fraction of the fusion antibody bound to EGFR was similar to that of its parent antibody; (2) formation of the steric shield effectively blocked EGFR binding as steric shield formation by transglutamination under two different experimental conditions reduced the fraction of the activatable antibodies bound to EGFR compared to the parent antibody; and (3) following steric shield release by protease cleavage of the tumor-sensing releasable moiety SEQ ID NO: 19 (matriptase or uPA), EGFR binding was restored to levels similar to that of parent antibody.

FIG. 12B shows representative SPR sensograms of EGFR binding to parent anti-EGFR antibody and activatable EGFR Ab 3 with shield in place, and with shield removed by protease matriptase. FIGS. 9B and 12B show bar graphs illustrating differences in binding activities of activatable EGFR Ab 3 and 4 to the target. EGFR, before and after shield moiety formation via Cys-Cys disulfide bridge, and before and after in vitro release of the steric shield via cleavage of the tumor sensing releasable moiety by protease. The results demonstrate: (1) formation of the steric shield effectively blocked EGFR binding as steric shield formation via Cys-Cys disulfide conjugation under two different experimental conditions and for two different polypeptide shield moiety sequences reduced the fraction of the activatable antibodies bound to EGFR compared to the parent antibody; (2) following steric shield release by protease cleavage of the tumor-sensing releasable moieties (matriptase or uPA), EGFR binding was restored to levels similar to that of parent antibody.

### Example 10. In Vitro Efficacy of Activatable EGFR Ab 2-4

The in vitro efficacy of activatable EGFR Ab 2, 3, and 4 was evaluated by cell proliferation assays. H292 human lung cancer cell line NCI-H292 (ATCC, USA) and human colon cancer cell line SW49 (ATCC, USA) were used because proliferation of H292 and SW48 cells is sensitive to EGFR inhibition. H292 and SW48 cells were maintained at 37° C. (5% CO₂) in RPMI-1640 medium (Gibco. USA) supplemented with 15% fetal bovine serum (FBS) (Hyclone, USA). For the proliferation assay of SW48 cell line, cells were seeded at 1680 cells per well in a 384-well plate under low serum conditions (RPMI containing 2% FBS). For the proliferation assay of H292 cell line, cell were seeded at 850 cells per well in a 384 well plate under low serum condition (RPMI containing 2% FBS) After 24 hrs, parent anti-EGFR antibody, activatable EGFR Ab 2, 3 and 4 with a shield in place, or matriptase-treated activatable EGFR Ab 2, 3 and 4 (activated activatable EGFR Ab) were each respectively added to the cancer cells at 2 fold serial dilutions. Cell viability was measured 96 hrs later using a CELLTITER-GLO™ kit (Promega) according to the manufacturer's instructions.

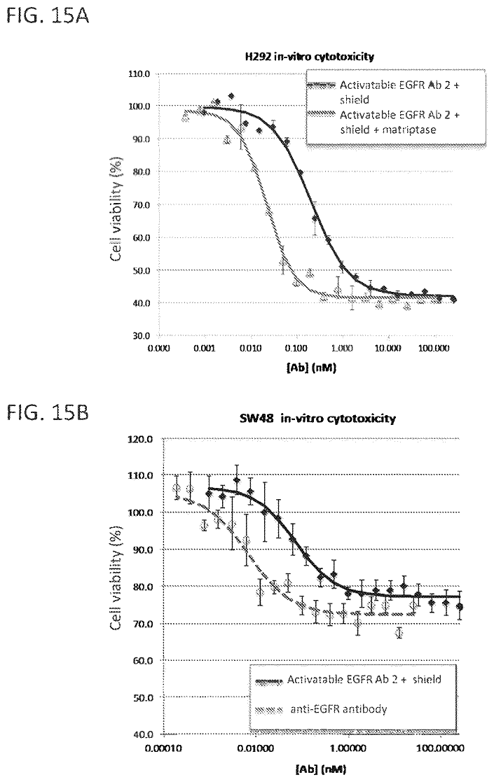

FIG. 15A shows representative results of H292 cell proliferation in the presence of activatable EGFR Ab 2 carrying a steric shield and active activatable EGFR Ab 2 with shield released by matriptase cleavage of the disease-sensing moieties. The data shows that the anti-proliferation activity of activatable EGFR Ab 2 carrying a steric shield is reduced relative to that of activated activatable EGFR Ab 2, demonstrating the shielding effect. Error bars represent standard errors of the mean (SEM).

FIG. 15B shows representative results of SW48 cell proliferation in the presence of activatable EGFR Ab 2 carrying a steric shield and EGFR Ab. The data shows that the anti-proliferation activity of activatable EGFR Ab 2 carrying a steric shield is reduced relative to that of the parent anti-EGFR antibody, demonstrating the shielding effect.

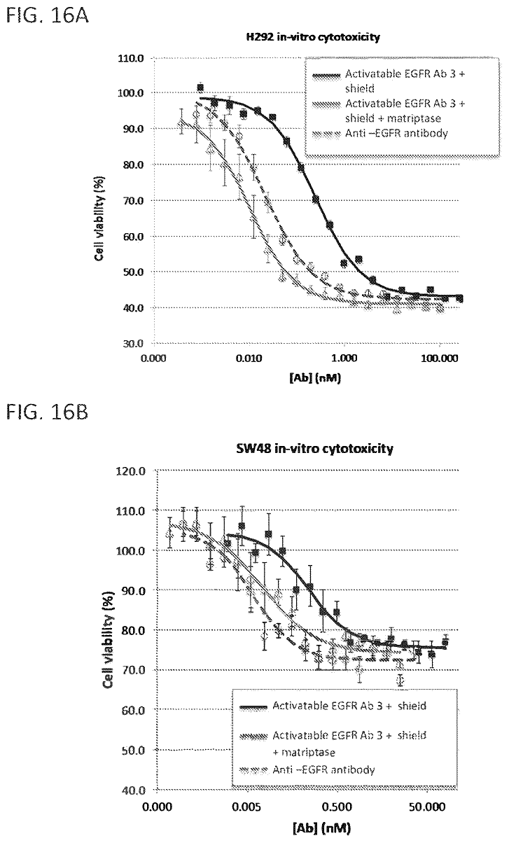

FIG. 16A shows representative results of 11292 cell proliferation in the presence of activatable EGFR Ab 3 carrying a steric shield, active activatable EGFR Ab 3 with shield released by matriptase cleavage of the disease-sensing moieties, and EGFR Ab. The data shows that the anti-proliferation activity of activatable EGFR Ab 3 carrying a steric shield is reduced relative to that of activated activatable EGFR Ab 3 and EGFR Ab, demonstrating the shielding effect. In addition, this data shows that the anti-proliferation activity of activatable EGFR Ab 3 with the tumor-sensing releasable moieties cleaved by matriptase is similar to that of the parent anti-EGFR antibody.

FIG. 16B shows representative results of SW48 cell proliferation in the presence of activatable EGFR Ab 3 carrying a steric shield, active activatable EGFR Ab 3 with shield released by matriptase cleavage of the disease-sensing moieties, and parent anti-EGFR antibody. The data shows that the anti-proliferation activity of activatable EGFR Ab 3 carrying a steric shield is reduced relative to that of activated activatable EGFR Ab 3 and the parent antibody, demonstrating the shielding effect. In addition, this data shows that the anti-proliferation activity of activatable EGFR Ab 3 with the tumor-sensing releasable moieties cleaved by matriptase is nearly identical to that of the parent anti-EGFR antibody.

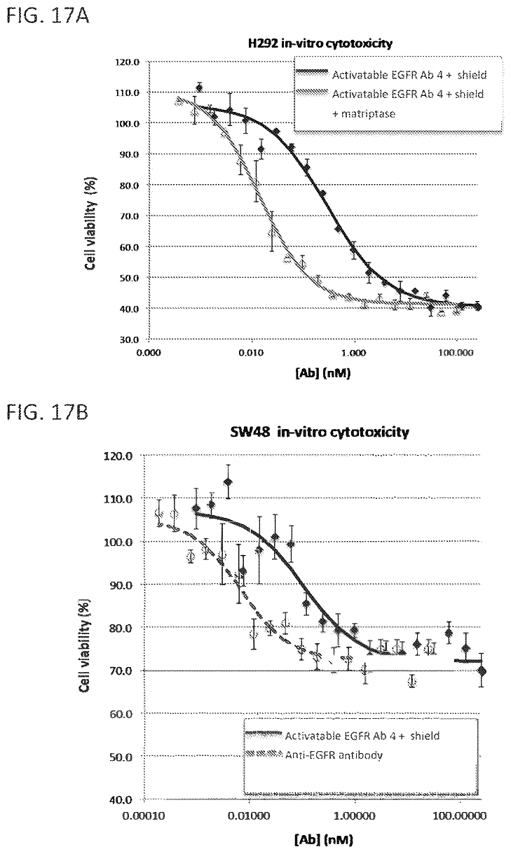

FIG. 17A shows representative results of H292 cell proliferation in the presence of activatable EGFR Ab 4 carrying a steric shield and active activatable EGFR Ab 4 with shield released by matriptase cleavage of the disease-sensing moieties. The data shows that the anti-proliferation activity of activatable EGFR Ab 4 carrying a steric shield is reduced relative to that of activated activatable EGFR Ab 4, demonstrating the shielding effect.

FIG. 178 shows representative results of SW48 cell proliferation in the presence of activatable EGFR Ab 4 carrying a steric shield and parent anti-FGFR antibody. The data shows that the anti-proliferation activity of activatable EGFR Ab 4 carrying a steric shield is reduced relative to that of the parent antibody, demonstrating the shielding effect.

The data in FIGS. 15A-17B suggests that activatable EGFR Ab 2-4 carrying a steric shield would have improved safety profile compared to the parent anti-FGFR antibody. At the same time, activatable EGFR Ab 2-4 would have comparable efficacy as the parent antibody after release of the steric shield mediated by cleavage of the disease-sensing releasable moieties (such as in the tumor environment).

### Example 11. In Vivo Efficacy of an Exemplary Activatable Anti-EGFR Antibody

The in vivo efficacy of activatable EGFR Ab 2 is assessed in a human 11292 lung xenograft model in nude mice. H292 xenografts are established by subcutaneous injection of 5×10⁶ (NCI-H292) cells mixed 1:1 in MATRIGEL® into the left flank of each mouse. Tumor cells are allowed to reach 200 to 400 mm³ for the study before randomization into three treatment groups, with 10 mice per group. Parent anti-EGFR antibody, activatable EGFR Ab 2 with an intact steric shield (i.e., treated with transglutaminase), or matriptase-treated activatable EGFR Ab 2 (i.e. steric shield released) is injected subcutaneously into the animals weekly at 25 mg/kg. Animals are sacrificed once the tumor volume reached 20) mm³ or at the end of the study (21 days).

At the 25 ng/kg dose, activatable EGFR Ab 2 and the parent anti-EGFR antibody show comparable efficacy at suppressing tumor growth in the 11292 model. This data shows that activatable EGFR Ab with intact steric shield can be activated in the tumor environment and achieve comparable efficacy to the parent anti-EGFR antibody.

### Example 12. Exemplary Activatable Anti-HER-2 Fab and Activatable Anti-Mouse CTLA-4 Fab Constructs with Polypeptide Shield Moieties

Exemplary anti-mouse CTLA-4 activatable Fabs (referred herein as “activatable mCTLA-4 Fab”) and an exemplary anti-HER-2 activatable Fab (referred herein as “activatable hHER-2 Fab”) were prepared. The corresponding heavy chain and light chain sequences of the activatable hHER-2 Fab and activatable mCTLA-4 Fab (and the corresponding fusion Fabs) are shown in Table 11. Sequences corresponding to the polypeptide shield moieties S1 and S2 are underlined. The parent anti-HER-2 antibody comprises a VH comprising the amino acid sequence of SEQ ID NO: 234 and a VL comprising the amino acid sequence of SEQ ID NO: 235. The parent anti-mouse CTLA-4 antibody comprises a VH comprising the amino acid sequence of SEQ ID NO: 238 and a VL comprising the amino acid sequence of SEQ ID NO: 239.

### Design of Activatable Fabs

In order to illustrate that the same shield moieties can form shield on antigen binding sites specific for different antigens, activatable hHER-2 Fab was designed to carry S1 and S2 shield moieties with the same sequences as the S1 and S2 shield moieties on anti-EGFR activatable EGFR Ab 2 (SEQ ID NOs: 142 and 143). The activatable hHER-2 Fab was designed such that the sequence of disease sensing moieties in the shield moieties S1 and S2 are the same, LSGRSDNH (SEQ ID NO: 19), which is substrate(s) of the same protease(s). Thus, in the presence of a specific protease, steric shields can be released from both the S1 and S2 disease sensing moieties. Anti-mouse CTLA-4 activatable mCTLA-4 Fab was designed to carry a S2 shield moiety (SEQ ID NO: 143) with the same sequence as S2 moiety on activatable EGFR Ab 2 and activatable hHER-2 Fab, and an S1 shield moiety (SEQ ID NO: 227) with the same association moiety, same number of amino acids, but different disease-sensing moiety compared to the S2 shield moiety on activatable EGFR Ab 2 and on activatable hHER-2 Fab. The activatable mCTLA-4 Fab was designed such that sequences of the disease sensing moieties in shield moieties S1 and S2 are different. PLGLAG (SEQ ID NO: 172) and LSGRSDNH (SEQ ID NO: 19). In this case, the presence of protease(s) specific for only one disease-sensing moiety (e.g., S1-specific) can release the steric shield by cleavage of only one disease-sensing moiety (e.g., S1 disease sensing moiety or S2 disease sensing moiety).

### Steric Shield Generation by Transglutaminase-Catalyzed Conjugation

Steric shields were formed on the activatable hHER-2 Fab, and the activatable mCTLA-4 Fab by treating the corresponding fusion antibodies with a transglutaminase under different reaction conditions. The following reaction parameters were used: transglutaminase (Ajinomoto, Japan) concentration range (0.5, 0.75, 2.0% wt/v), fusion antibody concentration range (0.05, 0.5 and 6 mg/ml), NaCl concentration 150. pH 7.4 and 8.5 and temperature of reaction (20, 37° C.) for 12 hr. Efficiency of transglutaminase-catalyzed steric shield formation was analyzed using reduced 4-12% Tris-Glycine SDS-PAGE gels (ThermoFisher. USA).

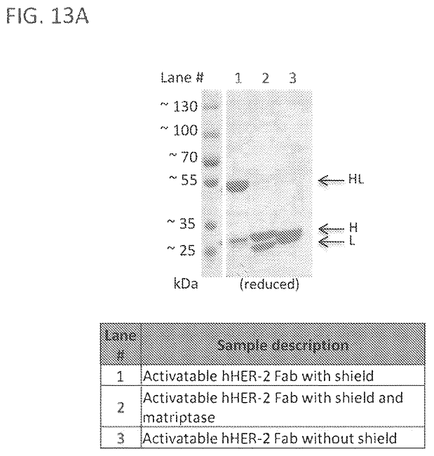

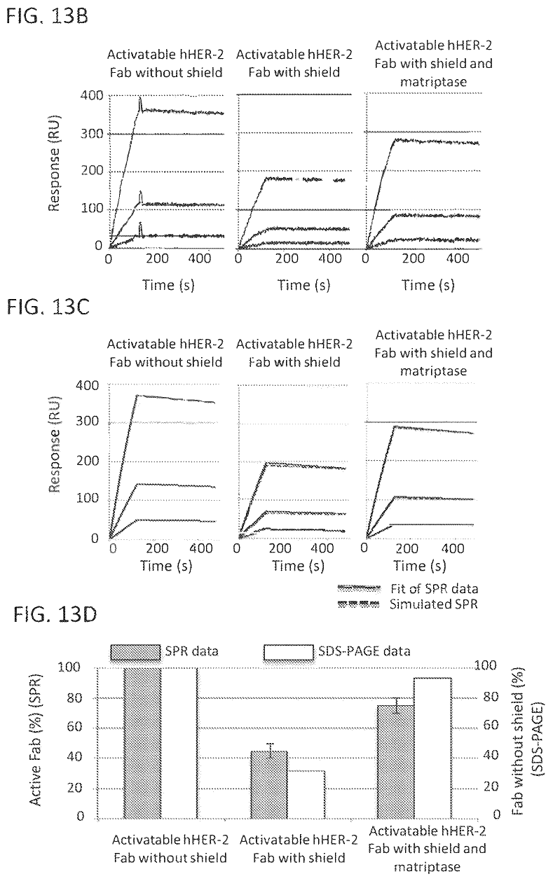

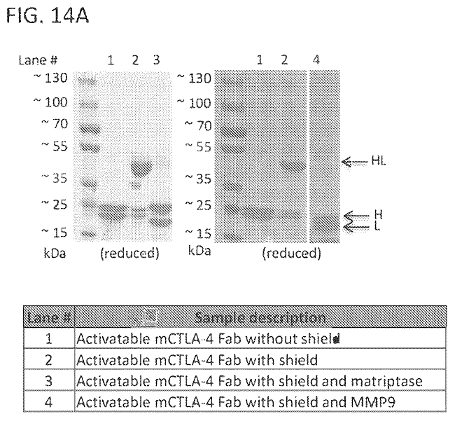

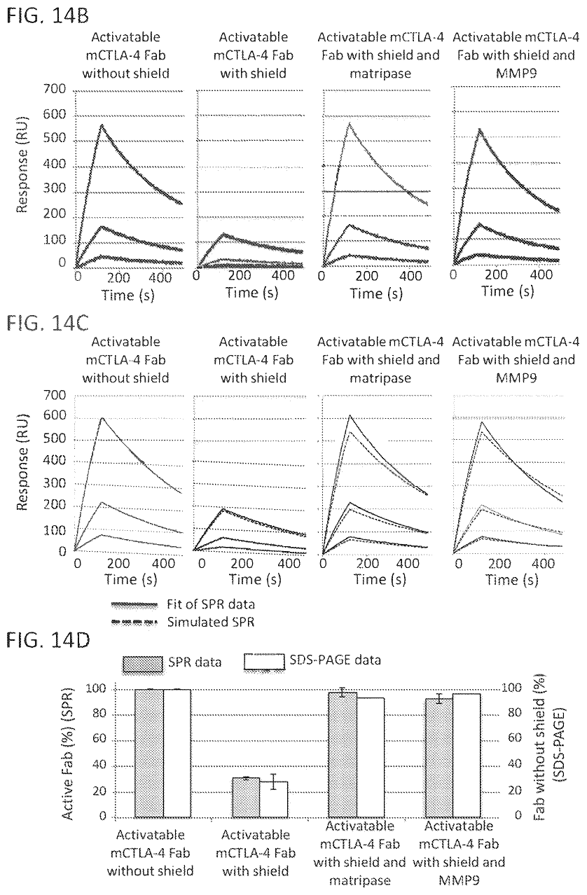

FIGS. 13A and 14A show representative results of a subset of tested conditions for activatable hHER-2 Fab and activatable mCTLA-4 Fab, respectively. Both activatable hHER-2 Fab and activatable mCTLA-4 Fab were designed with a C-terminal His-tag, which allowed separation of the product from transglutaminase using Ni-NTA affinity chromatography after the reaction was complete. After elution with 300 mM imidazole, activatable hHER-2 Fab and activatable mCTLA-4 Fab were buffer-exchanged into PBS pH 7.4 buffer. Various samples were run on a 4-12% SDS-PAGE gel in the presence of a reducing agent (TCEP) so that heavy chains (marked by letter H) and light chains (marked by letter L) in conventional Fabs would run as separate bands, both around ˜25 kDa. However, activatable antibodies having conjugated steric shield would yield a band corresponding to the conjugated heavy chain and light chain Fab species at about ˜50 kDa. Notably, in the presence of a transglutaminase, the heavy chain and light chain in the activatable Fab constructs were conjugated, and thus ran as the 50 kDa species (marked as H+L). Thus, this data clearly demonstrates that the polypeptide shield moieties fused to the N-termini of the heavy chain and light chain of activatable hHER-2 Fab and activatable mCTLA-4 Fab were conjugated by the transglutaminase, thereby providing a complete shield.

### In Vitro Removal of Transglutaminase-Conjugated Steric Shield by Protease Cleavage

In order to determine whether the activatable hHER-2 Fab and activatable mCTLA-4 Fab could be activated specifically in tumor tissues, n vitro experiments were performed using proteases matriptase and MMP9, which are up-regulated in cancers.

After steric shield formation, for in vitro shield removal, activatable hHER-2 Fab and activatable mCTLA-4 Fab with shield moiety in place were incubated with matriptase at a protease:antibody ratio of 1:100 (mole:mole) in 50 mM Tris pH 8.5, 0.01% Tween-20 buffer for 18 hrs at 37° C. In addition, in a separate reaction, activatable mCTLA-4 Fab with shield moiety in place was incubated with MMP9 protease in PBS. pH 7.4 with 10 mM CaCl₃. Effects of matriptase or MMP9-specific digestion were evaluated by running samples on reducing 4-12% Tris-Glycine SDS-PAGE gels (ThermoFisher) in the presence of the reducing agent TCEP, so that heavy (marked by letter H) and light (marked by letter L) chains would run as separate bands ˜25 kDa respectively, unless conjugated (marked as H+L, ˜50 kDa species).

Results are shown in FIGS. 13A (activatable hHER-2 Fab) and 14A (activatable mCTLA-4 Fab). The steric shield was first formed by incubation with a transglutaminase in separate reactions (sample lane indicated in the figure legend), followed by removal of the steric shields by cleavage of the tumor-sensing releasable moiety (SEQ ID NO: 19) by matriptase, and tumor shield moiety (SEQ ID NO: 172) by MMP9 (respective sample lanes are indicated in the figure legend). Also shown are control samples in which the activatable Fabs were not subject to the transglutaminase treatment, and thus a steric shield was never formed prior to matriptase or MMP9 treatment. As a result, the heavy (H) and light (L) chains ran as separate bands (˜25 kDa). Notably, in the presence of matriptase or MMP9, the fraction of conjugated heavy and light chains Fab (H+L, ˜50 kDa band) decreased, while the fraction of heavy (H) and light (L) chains that ran as separate bands (˜25 kDa) increased Thus, this data demonstrates that the transglutaminase-conjugated steric shield on activatable hHER-2 Fab and activatable mCTLA-4 Fab can be removed via cleavage of the disease-sensing releasable moieties by a disease-specific protease.

### Antigen Binding to Activatable Antigen Binding Fragment (Fab)

Binding of human HER-2 to activatable hHER-2 Fab and mouse CTLA-4 to activatable mCTLA-4 Fab before shield formation (i.e., conjugation of S1 and S2), after shield formation, and after protease-specific shield removal was assessed using a PROTEON™ XPR36 Surface Plasmon Resonance (SPR) Instrument (BioRad). Antigens, recombinant human HER-2-Fc (R&D Systems, USA) and recombinant mouse CTLA-4-Fc (R&D Systems. USA) were respectively attached to chips via Protein G, and binding of respective anti-HER-2 and anti-mouse CTLA-4 activatable Fabs was measured. Phosphate buffered saline (PBS, pH 7.4) supplemented with 0.01% Tween-20 was used as the running buffer.

FIG. 13B shows representative sensograms of activatable hHER-2 Fab without shield, with shield, and with shield removed when binding to its antigen, human HER-2. FIG. 14B shows representative sensograms of activatable mCTLA-4 Fab without shield, with shield, and with shield removed when binding to its antigen, mouse CTLA-4. The data demonstrates that the presence of the steric shield on activatable hHER-2 Fab and activatable mCTLA-4 Fab suppresses binding to their targets, i.e., HER-2 and CTLA-4 respectively; removal of the steric shield by cleavage of the tumor-sensing releasable moiety SEQ ID NO: 19 (a matriptase substrate) or SEQ ID NO. 172 (an MMP2/9 substrate) recovers antigen binding efficiency.

In order to estimate the fraction of the Fab population with shield in place in the binding experiments, we simulated SPR curves with different inactive fractions utilizing the experimentally determined parameters (kₙ, k_{d}, Rₘₐₓ) and determined the best fit across the concentration range. In this analysis, activatable Fab without shield is assumed to bind at 100% with the same binding kinetics, while Fab with shield does not bind to the target. Representative experimental sensograms are shown in FIGS. 13B and 14B. Simulated sensograms (dashed lines) overlaid on experimental SPR fits (black lines) are shown in FIGS. 13C (activatable hHER-2 Fab) and 14C (activatable mCTLA-4 Fab).

Results showing fractions of active (i.e. without shield) Fab species before shield formation, after shield formation, and after shield removal by proteases are shown as bar graphs in FIGS. 13D (activatable hHER-2 Fab) and 14D (activatable mCTLA-4 Fab). The gray bars are based on SPR data, and the white bars are based on integration of pixels of corresponding bands on SDS-PAGE gels (Image J software). Error bars are standard deviations from two to three data sets.

The results demonstrate: (1) formation of the steric shield effectively blocked antigen binding as steric shield formation by transglutamination of shield peptides in two different activatable Fabs, activatable hHER-2 Fab and activatable mCTLA-4 Fab, reduced the fraction of the activatable Fabs bound to their respective antigens; (2) the fractions of activatable hHER-2 Fab and activatable mCTLA-4 Fab carrying a shield (as estimated from analysis of SDS-PAGE gels) and the fractions of activatable hHER-2 Fab and activatable mCTLA-4 Fab that were not able to bind to their respective antigens (i.e., inactive species in the SPR experiments) are similar and positively correlated, which suggests that the presence of the shield prevents antigen binding; (3) following steric shield release by protease cleavage of the tumor-sensing releasable moieties (matriptase or MMP9), respective antigen binding was restored to levels similar to that of an activatable Fab with unconjugated polypeptide shield moieties; (4) S1 and S2 steric shield moieties having the same sequence and/or length can be conjugated to form a shield on activatable antigen binding domains specific for different antigens (e.g., HER-2, CTLA-4 and EGFR); (5) release of the conjugated shield moiety by cleavage of one but not both tumor-sensing moieties (i.e., matriptase or MMP9-mediated shield release on activatable mCTLA-4 Fab), does not hinder antigen binding.

1: An activatable antibody comprising an antibody comprising an antigen-binding domain (ABD), wherein the ABD comprises a heavy chain variable region (VH) and a light chain variable region (VL), wherein the N-terminus of the VH is fused to a first polypeptide shield moiety (S1), and the N-terminus of the VL is fused to a second polypeptide shield moiety (S2), wherein each of S1 and S2 is a polypeptide no more than about 50 amino acids long, wherein S1 comprises a first disease-sensing releasable moiety (DS1) and/or S2 comprises a second disease-sensing releasable moiety (DS2),
wherein DS1 and/or D2 is a peptide substrate for a tumor-specific protease selected from the group consisting of matriptase, urinary-type plasminogen activator, legumain, kallikrein-related peptidase-3, matrix metalloproteinase-2, matrix metalloproteinase-9 (MMP9), human neutrophil elastase, proteinase 3 (Pr3), cathepsin B, and cathepsin K, wherein association of S1 with S2 spans a length greater than about 35 Å and blocks binding of the ABD to its target, wherein the ABD does not specifically bind to S1, S2, or association thereof,
wherein S1 comprises a first association moiety (B) and S2 comprises a second association moiety (C),
wherein B comprises a sequence selected from the group consisting of: SEO ID NOs: 1, 53-62, 124, 127, 128, 129, 130, 131, and 132-139 and C comprises a sequence selected from the group consisting of: SEQ ID NOs: 2 and 74-75, or wherein B comprises a sequence selected from the group consisting of: SEO ID NOs: 2 and 74-75 and C comprises a sequence selected from the group consisting of: SEQ ID NOs: 1, 53-62, 124, 127, 128, 129, 130, 131, and 132-139, and
wherein B and C are conjugated to each other by an isopeptide bond.

2. (canceled)

3: The activatable antibody of claim 1, wherein S1 and/or S2 comprise at least 7 amino acid residues.

4-6. (canceled)

7: The activatable antibody of claim 1, wherein B and C are conjugated to each other by a transglutaminase.

8: The activatable antibody of claim 1, wherein one of B and C comprises the amino acid sequence of SEQ ID NO: 1, and the other comprises the amino acid sequence of SEQ ID NO: 2.

9: The activatable antibody of claim 8, wherein S1 comprises the amino acid sequence of any one of SEQ ID NOs: 140, 142 and 227, and wherein S2 comprises the amino acid sequence of any one of SEQ ID NOs: 141 and 143; or wherein S2 comprises the amino acid sequence of any one of SEQ ID NOs: 141 and 143, and wherein S1 comprises the amino acid sequence of any one of SEQ ID NOs: 140, 142 and 227.

10-34. (canceled)

35: The activatable antibody of claim 1, wherein S1 comprises DS1 and S2 does not comprise DS2.

36. (canceled)

37: The activatable antibody of claim 1, wherein S1 comprises DS1, and S2 comprises DS2.

38: The activatable antibody of claim 37, wherein DS1 is identical to DS2.

39: The activatable antibody of claim 37, wherein DS1 is different from DS2.

40: The activatable antibody of claim 1, wherein DS1 and/or DS2 comprises: any one of SEQ ID NOs: 19, 147-202, 204, and 240-241.

41-50. (canceled)

51: The activatable antibody of claim 1, wherein the antibody is a bispecific antibody.

52. (canceled)

53: The activatable antibody of claim 1, wherein the antibody is a Fab, a (Fab)₂, an Fv, or a full-length antibody.

54-55. (canceled)

56: The activatable antibody of claim 1, wherein the antibody is an antibody-drug conjugate (ADC).

57: The activatable antibody of claim 1, wherein the ABD specifically binds to EGFR, CTLA-4, PD-1, CD-71, PD-L1, HER2, CD3, a-4 integrin, a-V integrin, a-4-b-1 integrin, a-4-b-7 integrin, AGR2, Anti-Lewis Y, Apelin J receptor, APRIL, B7-H3, B7-H4, BCMA, BTLA, C5 complement, C-242, CA9, CA19-9, Carbonic anhydrase 9, CD2, CD3, CD6, CD9, CD11a, CD19, CD20, IL27, IL27R IL29, IL31, IL31R, Insulin Receptor, Jagged-1, Jagged-2, KIR, LAG-3, LIF-R, Lewis X, LIGHT, LRP4, LRRC26, CD22, CD24, CD25, CD27, CD28, CD30, CD40, CD40L, CD41, CD44, CD44v6, CD47, CD51, CD52, CD56, CD64, CD70, CD71, CD74, CD80, CD81, CD86, CD95, CD117, MCSP, Mesothelin, MRP4, MUC1, Mucin-16, Na/K ATPase, Neutrophil elastase, NGF, Nicastrin, NOTCHL, NOTCH2, NOTCH3, NOTCH4, NOV, OSM-R, CD125, CD132, CD133, CD137, CD138, CD166, CD172A, CD248, CDH6, CEACM5, CEACAM6, CLAUDIN-3, CLAUDIN-4, cMet, Collagen, Cripto, CSFR, CSFR-1, CTLA-4, CTFG, CXCL10, CXCL13, CXCRL, CXCR2, OX-40L, OX-40R, PAR2, PDGF-AA, PDGF-BB, PDGFRa, PDGFRb, PD-1, PD-L1, PD-L2, Phospahtidyl-serine, PIGF, PSCA, PSMA, RAAG12, CXCR4, CYR61, DL44, DLKI, DLL4, DPP-4, DSGI, EGFR, EGFRVIII, Endothelin b receptor (ETBR), ENPP3, EPCAM, EPHA2, EPHB2, ERBB3, F protein of RSV, FAP, FGF-2, FGF-8, FGFR-1, FGFR-2, FGFR-3, FGFR-4, Folate receptor, RAGE, SLC44A4, TNFa, STEAP1, STEAP2, TAG-72, TAPA1, TGFb, TIGIT, TIM3, TLR2, TLR4, TLR6, TLR7, TLR8, GAL3STI, G-CSF, G-CSFR GD2, GITR, GLUTL GLUT4, GM-CSF, GM-CSFR, GPIIb/IIIa receptor, Gp130, GPIIB/IIIA, GPNMB, GRP78, HER-2/neu, HGF, hGH, HVEM, Hyaluronidase, ICOS, INFa, INFb, INFg IgE, TLR9, TMEM31, Sphingosine 1, Phosphate, TNFR, TNFRS12A, TRAIL-R1, TRAIL-R2, Transferrin, Transferrin Receptor, TRK-A, TRK B, uPAR, VCAM-1, VEGF, VEGF-A, IgE Receptor, IGF, IGF1R, IL1B, IL1R, IL2R, IL4, IL4R, IL6, IL6R, IL11, IL12, IL12p40, IL12R, IL12Rb1, IL13, IL13R, IL15, IL17, IL18, IL21, IL23, IL23R, VEGF-B, VEGF-C, VEGF-D, VEGFR1, VEGFR2, VEGFR3, VISTA, WISP-1, WISP-2, or WISP.

58. (canceled)

59: The activatable antibody of claim 1, wherein disruption of DS1 and/or DS2 unblocks the binding of the ABD to its target.

60-66. (canceled)

67: A pharmaceutical composition comprising the activatable antibody of claim 1 and a pharmaceutically acceptable carrier.

68-70. (canceled)

71: The activatable antibody of claim 1, wherein S1 and/or S2 further comprises a glycosylation peptide sequence.

72: The activatable antibody of claim 71, wherein the glycosylation peptide sequence comprises the amino acid sequence of SEQ ID NO: 18.
